SEQUENCE LISTING (179 sequences)

SEQ ID NO: 1 - DNA, 34 bp, Artificial Sequence
  Synthetic Polynucleotide
gaagttcctattctctagaaagtataggaacttc

SEQ ID NO: 2 - DNA, 61 bp, Artificial Sequence
  Synthetic Polynucleotide
aaacgatatcagacatttgtctgataatgcttcattatcagacaaatgtctgatatcgttt

SEQ ID NO: 3 - DNA, 39 bp, Artificial Sequence
  Synthetic Polynucleotide
gagtttcattaaggaataactaattccctaatgaaactc

SEQ ID NO: 4 - DNA, 33 bp, Artificial Sequence
  Synthetic Polynucleotide
ggttgcttaagaataagtaattcttaagcaacc

SEQ ID NO: 5 - DNA, 31 bp, Artificial Sequence
  Synthetic Polynucleotide
ttgatgaaagaataacgtattctttcatcaa

SEQ ID NO: 6 - DNA, 34 bp, Artificial Sequence
  Synthetic Polynucleotide
ataacttcgtatagcatacattatacgaagttat

SEQ ID NO: 7 - DNA, 34 bp, Artificial Sequence
  Synthetic Polynucleotide
tcaatttctgagaactgtcattctcggaaattga

SEQ ID NO: 8 - DNA, 34 bp, Artificial Sequence
  Synthetic Polynucleotide
ctcgtgtccgataactgtaattatcggacatgat

SEQ ID NO: 9 - DNA, 34 bp, Artificial Sequence
  Synthetic Polynucleotide
aataggtctgagaacgcccattctcagacgtatt

SEQ ID NO: 10 - DNA, 32 bp, Artificial Sequence
  Synthetic Polynucleotide
taactttaaataatgccaattatttaaagtta

SEQ ID NO: 11 - DNA, 21 bp, Artificial Sequence
  Synthetic Polynucleotide
cagcttttttatactaagttg

SEQ ID NO: 12 - DNA, 21 bp, Artificial Sequence
  Synthetic Polynucleotide
ctgcttttttatactaacttg

SEQ ID NO: 13 - DNA, 21 bp, Artificial Sequence
  Synthetic Polynucleotide
atcctttaggtgaataagttg

SEQ ID NO: 14 - DNA, 21 bp, Artificial Sequence
  Synthetic Polynucleotide
gcactttaggtgaaaaaggtt

SEQ ID NO: 15 - DNA, 38 bp, Artificial Sequence
  Synthetic Polynucleotide
ccccaactggggtaacctttgagttctctcagttgggg

SEQ ID NO: 16 - DNA, 34 bp, Artificial Sequence
  Synthetic Polynucleotide
gtgccagggcgtgcccttgggctccccgggcgcg

SEQ ID NO: 17 - DNA, 48 bp, Artificial Sequence
  Synthetic Polynucleotide
ggtttgtctggtcaaccaccgcggtctcagtggtgtacggtacaaacc

SEQ ID NO: 18 - DNA, 38 bp, Artificial Sequence
  Synthetic Polynucleotide
ggcttgtcgacgacggcggtctccgtcgtcaggatcat

SEQ ID NO: 19 - DNA, 26 bp, Artificial Sequence
  Synthetic Polynucleotide
ttatccaaaacctcggtttacaggaa

SEQ ID NO: 20 - DNA, 28 bp, Artificial Sequence
  Synthetic Polynucleotide
cgttcgaaatattataaattatcagaca

SEQ ID NO: 21 - protein, 405 aa, Unknown
  Archaeal BJ1 virus
MTDQPGNAIDRNVERCQECDEMSEADAEAILDAHRQMELLGASRLSKSHHSDVLMRAVKMAREVGGLANALEEREATEEIVRWIQRTYDNEETNRDYRKCLRAFGRHATRSEEPPDSIAWVPAGYSNTYDPAPDPGEMFRWQKHVKPMVDASSNVRDEALVALCWDLGPRTSELHELQVSNITEADYGLRVTIENGKNGSRSPTIVKATPYVRDWLERHPGDRDDYLWSRLNSPKRVSRNYLRDTLKRLASNAAMDPPATPTPTQFRKSSASYLARQNVNQTFIEDHHGWVRGSDKAARYVAVFDDSSDDAIASAHGVDVDITDDTPSMQECVRCDELNEPDRSRCRRCGYALTQEAVETEETREERFNKQLAMLDKENAMRLVEVMDALDDPEVLAALDEVASR

SEQ ID NO: 22 - protein, 453 aa, Natronobacterium magadii
MTDADPREEVDTLRDRLRSSGEDARYVQFEADRRHLLKFSDNIRLVPSEIGDHRHLKLLRHCCRMAALVPPPTVEDFKDNDEAADAGIVDEDDVDDLLEEHGLLGLTLEYRAAAEGVVRWINEEYANEHTNQDYRTALRSFGRYRLKRDEPPESLTWIPTGTSNDFDPVPSERDLLTHDDVRAMIEEGSRNPRDKALLAVQFEAGLRGGELYDVRVGDVFDGEHSVGLHVDGKEGERSVHLITSVPYLQQWLTSHPAPDDDQAWLWSKLSSAERPSYATFLNYFKNAAARVDVTKDVTPTNFRKSNTRWLILQNFSTARIEDRQGRKRGSEHTARYMARFGEESNERAYAQLHGLDVEANETEEVAPPVPCPRCGEDTPSDRDFCIHCHQSLDFEAKELLDEVREVLDNRSIEAEDPEDRREFVSARRTLEEKPHVMDKDDLHEFASSLSAED

SEQ ID NO: 23 - protein, 435 aa, Haloferax gibbonsii
MPSDPKQSVATLRKKLRNGTRGGCDRDRELLLDFSDELRLLREDYGHYRHEKLLRHNVRISENAETCLHETLVRERDGDADDEETFYDAKDAAKVVVRWIHGTYDIEDGSQETNRDYRVAFRLFAKHVTRGDDIPDTHSWISTKTSRDYQPEPDEADMLDLERDVEPMIEAARNPRDKALIALQFEGGFRGGELYDMRVEDITDGKHSLKVRVDGKRGEHDVHLIVAVPYVKRWLAEHPGDHDDYLWTKLTEPERFSYTRFLQCFKAAGKRAEIRKPVTPTNFRKSNAYWLSTREKSQAFIEDRQGRARGSPVISRYVAKFSGETQEIQYAAMHGLEAVETETKELAPVTCPRCEKETPRERGFCIHCNQSLDIESKELLDRIGTAIDDKVVEADDADTRRDLLRARRTLDERPAMMDTEELHELASRFSLSDEA

SEQ ID NO: 24 - protein, 403 aa, Unknown
  Halobiforma nitratireducens
MATTPRKRIDSLRDRAETGGDIGDRDRELLLEFSDTLDLLAQEYSDHRHEKLLRHCVIMAEELEDNTLAAALDNRDATETIVAWINRNYDNEETNRDYRSAIRVFAKRVTDGSECPPTVDWVPTGTSRNYDPSPDPREMLKWEDDAVPMIDECFNARDAAMIALQFDAGLRGGEFKSLTVGDIQDHDHGLQVTVEGKQGRRTIMLIPSVPYVNRWLDDHPDRDDPDAPLWSKITKVEGISDRMVSKVFDEAAGRAGVEKPVTLTNFRKSSAAFLASRNLNQAHIEEHHGWVRGSDVAARYISVFGEDSDRELAKLHGVDVSEDEPDPIAPLECTRCGRETPRDEPLCVWCGQAMDPQAAAELDEADDREAEALAELPPEKAKRLLEVADVLDDPEIRSTLLDR

SEQ ID NO: 25 - protein, 412 aa, Unknown
  Haloarcula amylolytica
MPVARGTVYMTDNPASAVDTMVDRLEDGHYDISDADRDLLLDLDRQIRLLGPSEFSDHRHEFLLRRGLIIAKRVGGLADGVDDREAAEDIVQWINTEQTGSPETNKDYRVAFRTIGKIVTDGDEYPDAVEWVPGGYPDNYDPAPNPATMLDWADDIQPMLDACLNSRDRALVALAWDLGPRPGELYDLTPGDIVDHDYGLQVTLNGKNGRRSPVLVPSVPYVRRWLDDHPGGDTDPLWCKLSSPESISNNRVRDALKDVADRAGVDKTVTPTHFRKSSASYLASQGVSQAHLEEHHGWTRGSDIASRYIAVFDDASEREIARAHGLDVEADEPDSVGPIVCPRCEQKTPREKDACVWCGQVLSQSAAEEAERQRQDAMDSMVAADSDLAEAIATVEAEIGDDVSIRIEGLDE

SEQ ID NO: 26 - protein, 390 aa, Methanosarcina acetivorans
MSIHEYYTDIWLPKLEEKIRTADYPKRNRDLILKFETYLFSEGLKSLRVLKYLFVLDKIASGSSVSFSKMNEHHVQKIIADFERSELAASTKRDYKVIIRRFFKWLKGDKSPAAWIKVSKKVSDQKLPEYMITEDEVKRMIEAASNARDKAIIALLYDSGCRIGELGGVKIKNITFDQYGAVVVVSGKTGARRVRVTFAASYLAAWLDVHPYKEKSEAFVFINLEGVKKGEQMQYQAFQYTLKKIAKAAGIEKRIHLHLFRHSRSTELAQYLTEAQMEEHLGWAQGSEMPRTYVHLSGKQIDDAILGIYGKKKKEDTMPKLTSRICTRCKKENGPTSSFCAQCGLPLDPQAVQEVQVREDAMAQILEQLMKNKELRDLWNVAAEGKSSES

SEQ ID NO: 27 - protein, 423 aa, Unknown
  Halobellus rufus
MSDSDQIERLRERVRNSPTICDADKETLLTFSDELEFLDVEYTDVRHIKLLQHCILLAGDSEKYTTEELPDVALTSTFGSKDAVKDLGRWIRRNYDNEETKRDYRIALRMLGKRVTEGDDIPEPLQLLSAGTPRSYDPTPDPAKMLWWEDHIEPMIKNAHHLRDKAAIAVAWDSGARSEEFCGLRVGDVSDHEHGMKISVDGKTGERSFLLTTATSYLLQWLNVHPASNDPTAPLWCKLNAPEDTSYRMKLKMLKKPARRAGIEHTDITFRRMRKSSASYLASQNVNQAHLEDHHGWKRGSNIASRYIAVFGEANDREIARAHGVDVQTEEHEPLAPVTCTRCRNETPRNESFCVWCGQAMEHGAVEELEAEKREARIELLRIAREDPTLLDEIDRLEQVVGFVDSNPSILREARDFVDASAD

SEQ ID NO: 28 - protein, 397 aa, Methanosarcina mazei
MFKLADAENFLKSEELSECNREILSKYFRYLRHEGNSERTALNHMENMIWIAKALHECDLGKLAEDDLYLFFDALENYTYTDRAGKVKKYSEPTKETRKVSLKKFLKWNKNYELHEKIKCKRLKGKKLPEDIKCKEDIVKMIEAGSNSRDRAIIACFYESGARRGEQLSVKLKNVELDEYGAVITFPEGKTGARRVRLIFSAPYLREWLDDHPRKDDRDAPLWCTLDKNAGHMSVTGLVNVFNRCGEKAGIEKKVNPHSFRHDRATHLAANFTEQQLKMYLGWSPTSTQPATYVHLSGKNMDDAVLKMYGIKKAEDDPEFLKPGICPRCRELTTVNAKFCYKCGLPLTQEAATTLETIKTEYMQLSDLDEIREMKNALKQELEEISKLKEMMLKAGK

SEQ ID NO: 29 - protein, 415 aa, Unknown
  Haloarcula sp. CBA1127
MTRNADRRIENLQERIERAEEMSGDDQNVLQAFDNRLALLGSQYGKERREKLLRHCVRIAEEVGGLADSLDDKRAAEDIVRWIHDTYDNEESNRDYRVAFRMFGKHVTDGDEIPDSISWVSATTSKDYNPMPNPAKMLWWEEHILPMLDECRHARDKALIAVAWDSGARSGELRNLTVGDVSDHKYGLRISVDGKKGERSITLVPSVPHLRQWLNVHPGKDQPDAPLWSKLSKPEDISYQMKLKILKKHARKAGIDHTEVTFTQMRKSSASYLASDGVNQAHLEDHHGWDRGSDVASRYVAVFGDANDRAIAQAHGVDVEEDESDPIAPVTCPRCRNETPRDEPTCVWCSQAMDAAAVEEIEREQKEIRSELLQIAHDDPDFLDNLDRVERFIELGDENPEILREARAFADATES

SEQ ID NO: 30 - protein, 415 aa, Unknown
  Haladaptatus sp. R4
MTADPAGSIERLRNRVERSDTITPQDRENILAFSNRMALLRSEYSDQRHEKLLGHITRMAEQIEDISDALDDRKKAEDVVRWINRNYDNEETNKDYRIAFRVFAKRVTDGDDTPDSIDWIPSGYSNNYDPAPNPKNMLRWEGDILPMVKGTRNSRDAALVTVAWDSGARPGELQSLTVGDVTDYKHGLQVTVEGKTGQRTVSLIPSVPYLQRWLTDHPDSGDPNAPLWSKLSSPDQLSNRMLRKALNSAADRAGVKKPVNLTNFRKSSASYLASQNVNQAHLEDHHGWTRGSKVAARYVSVFGGDSDREIARAHGLDVGEDEPDPIAPLECPRCKRETPRQEEFCVWCGQAVEPGAIETMENDQRETRAALLRLAQEDPKLLDRVEQLQDVMALTDEHPDLLPDAQRFVNTLRED

SEQ ID NO: 31 - protein, 414 aa, Unknown
  Haloterrigena daqingensis
MPDIRKQITSLQDRIERSNDISEKDKQLLLAFSDEIDLLKSKYSDHRHNKLLRHCTIMAEEVGGLSEALEDPGAAKGLVRWIHRNYNNEYTNHDYRTALRVFGQRVTEGEDYPPGIEWIPSGTSSSHDPVPDPADMLEWETDILPMVDATRNSRDAALITVAFDAGPRADELRTLSIGDISDTEHGLRIWVDGKTGQRSVDLIPSVPYLKRWLSDHPASDDSTAPLWSKLNSPEGISYRQFLNCLKDAAKRAGVTKSVTPTNLRKSNATYLARKGMNQAFIEDRQGRKRGSDATAHYVARFGTDSEAEYARLHGLEVEEEEPEPIGPVKCPRCSKETPRHESSCVWCNQVLEYDAIDSIEDAQRDIRDVVLQFARDDPEILTDFQRNRELMDLFESNPDLYEEAQEFVESLPDE

SEQ ID NO: 32 - protein, 417 aa, Unknown
  Halolamina rubra
MTDQPKTAIKRNVERCRERDGLGDADAEAILDAHHHMELVGNAGVSDSHHSDVLMRAVKIARETEPGTLAAALEDRDAAEDVVRWINRTYDNPETNRGYRQAFRAFGRHSLGVDELPECLDWVPAGYPSNYDPAPDPAQMLRWDDHIKPMLEGCNNVRDEALVALCWDLGPRTSELHELQVGNISEGDYGLTVTIENGKNGSRSPTIVRSVPFVRDWLERHPGDRDDYLWTRMDRPERVSRNYLRDALKNAARRVDLDLPATPTPTRFRKSSASYLASQNVNQAFLEDHHGWVTGSDKAARYITVFSDQSDRAIAEAHGVDVDVEDDGPDMVECVRCEALNDADRSRCRQCDQVLSQEAAEQEALVDRVLSRLDDQLLEADDRDERAELLEGKQVVEERRSDLDVDALHQLLSSGDA

SEQ ID NO: 33 - protein, 349 aa, Unknown
  Rahnella sp. WP5
MGNLSPTNQTLPAIQAEEDVLARLKEFVQDKEAFSPNTWRQLMSVMRICHRWSIENSRSFLPMLPADLRDYLNWLQENGRASSTIATHGSLISMLHRNAGLIPPNTSPLVFRAVKKINRVAVVTGERTGQAVPFRLEDLLELDALWSDSISPRHKRDLAFLHVAYSTLLRISEIARLRVRDISRATDGRIILNVSYTKTIVQTGGLIKSLNSQSSRRLTEWLSVSGINSEPDAFLFCPVHRSGSATLSVTRPLSTPAIESIFAQAWHTIGAGEPIIPNKGRYAAWTGHSARVGAAQDMAGRGYAVAQIMQEGTWKKPETLMRYIRNLQAHEGAMTDIMEKSTQNHNNTK

SEQ ID NO: 34 - protein, 349 aa, Unknown
  Erwinia gerundensis
MTDSLPAPLPLHALSADADISARLAEFVRDKDAFSPNTWRQLLSVMRICFSWSQQNGRSFLPMSPDDLRDYLTHLQEIGRASSTISTHASLISMLHRNAGLVPPNTSPAVFRTMKKINRVAVIAGERTGQAVPFRLNDLMALDRCWVNATRLQDLRNLAFLHIAYGTLLRVSELARLRVRDVTRAEDGRIILDVAWTKTIVQTGGLIKALSALSTRRLEAWIAAAGLAREPDAFLFCRVHRCNKALLTEEAPLSTPAIEAIFSHAWQTIGPAEPARANKSRYRGWSGHSARVGAAQDMAKQGYAVAQIMQEGTWKKPETLMRYIRNIDAHQGAMVDLMERLRPDAESNN

SEQ ID NO: 35 - protein, 337 aa, Unknown
  Pantoea latae
MNALVPLSPSDDDLAQRLREFVQDKEAFAPNTWRQLMSVMRVCHRWASANNRTLLPMSPEDLRDYLSYLQSIGRASSTIGTHQSLISMLHRNAGLVPPSTSPLVSRAVKKINRVAVVSGERTGQAVPFRLSDLQKVEAAWAETPSLRNMRDLAFLHVAYSTLMRISEVSRFRVGDVMRAEDGRIILEGSWTKTILDAGSLIKALGSKSSAVVTKWIVASGLINEPDAFLFSPVHRSGKVMVAIDEPMSTPALKSIFTRAWEAAGYTDTAKPNKNRYRRWSGHSARVGAAQDLARKGYSVPQIMQEGTWKKPETLMRYIRYVEAHKGAMVDLMENQDE

SEQ ID NO: 36 - protein, 391 aa, Citrobacter freundii
MLQNEKYSGFPKNRVNFIKNLTDYTNVMVVFRNESLLVPVHLRDMPMTNLPVNQTESPLLITADKYDERVAENLHMFFVDREAASENTWAQMKSVLRSWGLWCKQFNKVWLPADPADVREYLIYLRETLGRKKNTIAMHKSMINKIHREAGLALPASHILVTRGMKKISRQAVLSGERVEQAIPLHLDDLFQLAEITQASGKMQQLRDLAFLGVAYNTLLRMSEVARLRIGDIQFQRDGSATLDVGYTKTIKDELGVVKVLAPDVAGWLRNWLNASGLTDESTFIFGKVDRYGNAHPAVKPMAGKNIEKIFAKAWEAVKGAPLESSRYRTWTGHSPRVGAAQDMALKGTELTQIMHEGTWKRPEQVMSYIRYIDANKSVMLDIVNSQRMKR

SEQ ID NO: 37 - protein, 342 aa, Unknown
  Pantoea septica
MNEFSGFTGVALSGAAGDDLTAKLTAFVRHREAFSPNTWRQLLSVMRICWRWSQENHRSFLPMLPEDMQDYLFHLQATGRSTSTISVHAALMSMLHRNAGLVPPTVSPDVVRAKKKINRTAVVSGERIGQAVPFCRPDLNRLDKLWKHSPRLQHLRDLAFMHVAYSTLLRMSELSRLRVRDITRAADGRIILDVGWTKTILQSGGIVKALSARSSERLMEWISASGLADEPDAILFCPVHRSNKITTFTTAPMSAPCLEDIWRRARRQAGDAPRVKTNKGRYSSWSGHSARVGAAQDMARKGISIAQIMQEGTWTQTQTVMRYIRMVEAHKGAMIGLMEEDS

SEQ ID NO: 38 - protein, 343 aa, Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 39 - protein, 380 aa, Unknown
  Pseudomonas protegens Pf-5
MGSITVRKRKDGSAAYTAQIRIMQKGVTVYQESQTFDRKTTAQAWIRKREAELHEPGAIERANRSGVSVKEMIDQYLKQYEKLRPLGKTKRATLNAIKESWLGDVTDAELTSQKLVEYAVWRMETFGIQAQTVGNDLAHLGAVLSVARPAWGYDVDPHAMSDARSVLRKMGAVSRSRERNRRPTLDELDRILTYFEQMRDRRRQEIDMLRVIVFALFSTRRQEEITRIRWDLLNESEQSALVTDMKNPGQKYGNDVWCHMPDEAWRVLQSMPKVADEVFPYNSRSVSASFTRACNFLEIEDLHFHDLRHDGVSRLFEMGWDIPKVASVSGHRDWNSMRRYTHLRGNGDPYAGWQWIERVISGPVIEAQVRVKRRAAGRAP

SEQ ID NO: 40 - protein, 358 aa, Burkholderia gladioli
MGTIVPRKRKDGSIGYTAQIRLKVKGKVVHTEAKTFDREPAASAWIKKRERELSQPGAIEGAKREDPTLGEVIARYIREDKRGIGRTKKQVLETIRGKDIAERPCSELRSADYIQFARSLDVQPQTVGNYMSHLGAIVRIARPAWGYPLAESEFDDAMVVGKRLGLTGKSVARDRRPTPDELNRILEYYTEMAKRERAELPMRELIVFALFSTRRQEEITTIRVEDFEGDRVLVRDMKHPGQKKGNDTWCDVPPEAARVIEAVRPKSGPIFPYNHRSISASFTKACAFLSIDDLHFHDLRHEGASRLFEMGLNIPHVAAVTGHRSWSSLKRYTHLRHVGDRWARWAWLDRVAPLQEQS

SEQ ID NO: 41 - protein, 382 aa, Acinetobacter baumannii
MGSITARKGADGNVSYRAAIRINKKGYPAYSESKTFYSKKVAENWLKKREVEIQENPDILFGKEQLIDLTLSDAIDKYLDEVGSEYGRTKRYALLLIKKLPIARNIITKIHSTHLAEHVALRRRGVPNLGLEPIATSTQQHELLHIRGVLSHASVMWGMDIDLSSFDKATAQLRKTRQISSSKVRDRLPTNEELVTLTKFFAERWKLNKYGTKYPMHLVIWFAIFSCRREAELTRLWLQDYDSYHSSWKVHDLKNPNGSKGNHKSFEVLEPCKTIVELLLDNEVRSRMLQLGYDERLLLPLNPKSIGKEFRDACKMLGIEDLRFHDLRHEGCTRLAEQSFTIPEIQKVSLHDSWSSLQRYVSVKSRRNVIQLEEVLRLIDET

SEQ ID NO: 42 - protein, 387 aa, Kingella oralis
MGSIVKRINPSGKTVYRAQIRIDRAAYPKYAESRTFSERRLAAAWLKKREAELEANPELLYYGGKKQTIPTLAQAIERYFSEPAATEFGRTKTATLKFLSGYPIAKLPLDKIRRADIAAHINQRRDGWGGFLPVKPQTVNNDLQYIRSMLKHAHFVWGLNVNWAEIDLAIEGARRARLIGKSEERMRLATAQELQALTTHFYQQWTTRPNSTKFPMHLIMWFAIYSCRREAEITRLAWVDYDKTAGDWLVRDLKSPSGSKGNHARFLVNDKLRQVIAAFRQPEIQNRLKWREMQPETWLIGGDSKSISASFTRACKLLGIEDLRFHDLRHEGATRLAEDGLTVPQMQQITLHQSWKTLQRYVNLATRPRENRLDFADALAVAQQKAA

SEQ ID NO: 43 - protein, 357 aa, Unknown
  Martelella sp. AD-3
MGTITARKKKKSGLIVYTAQIRITRKGKTVHSESQTFDRKKLAVAWMNKREGDLLEPGGLERAKHGNVTLADVIDQYIRENAAPMGRTKAQVLRTLKGYDIADLPCEEITSAHIIALARELSIDKKPQTVANYLSHLSSVFAIARPAWGYPLDRQAMQDGVIVAKRLGMTSKSRQRDRRPTLEELGRILTFFRRRSIQAPQSMPMDEIVLFALFSTRRQDEICRITWADLDAQNSRVLVRDMKNPGQKIGNDNWCDMPAPAMAVIRRAAQKDERIFPYAPESISANFTRACRLIGIEDLHFHDLRHEGISRLFEIGYNIPHAAAVSGHRSWVSLKRYSHIRQRGDKYEDWEWMPDTA

SEQ ID NO: 44 - protein, 356 aa, Unknown
  Afifella pfennigii
MGTITARKRKDGSVGYRARVRVMRDGMTYHETETFDRRPAAAAWMKKRERELSRPGAIPAAKFDDPTLAKAIDRYIEESVKEIGRTKAQVLRAIKKHPIVEMPCSTIKSKDIIEFLQSLTSQPQTVGNYASHLAAVFAIARPMWDYRLDEREMKDAITVARRLGIISRSLQRDRRPTLDELDKLLAHFIERRKKAPQALPMHKVIVFALFSTRRQEEITRIAWKDFQKEHKRVLVRDMKHPGEKLGNDTWVDLPSEAIQIIESMRKSKPEIFPYSTDAITANFTRACKLLDIENLHFHDLRHEGISRLFEMGWNIPHVAAVSGHRSWVSLKRYTHIRETGDKYAGWGGLRLAVSTK

SEQ ID NO: 45 - protein, 382 aa, Unknown
  Acinetobacter sp. MN12
MGSVTARKGTDGSVSYRAAIRINRKGYPVYSESKTFHSKKMAENWLKKREVEIQENPDILLGKEKHIDLTLADAIDKYLEEVGSEYGRTKRYSLLLIKKFPIARNIITKIKSVHLADHVALRKAGIPLLKLDPISTSTQQHELLHIRGVLAHASVMWDIDIDLNSFDKATAQLRKTRQISSSKKRDRLPTNEELIALTKYFVERWKLNKHGTKYPMHLVIWFAIFSCRREAELTRLSLDDYDQYHSSWKVHDLKNPNGSKGNHKSFDVLDPCKEMIKRLKQSEVRERMLRLGHDENLLLPLNPKSLGKEFREACKMLGIDDLRFHDLRHEGCTRLAEQSFTIPEIQKVSLHDSWSSLQRYVSVKARRSVMQLEDVLRLIDET

SEQ ID NO: 46 - protein, 383 aa, Eikenella corrodens
MGTITKRTNPSGAVVYRAQVRIKKAGAPAYNESKTFTKKALAAEWLKRREAEIEANPDLIFGIQKMRMPTLAAAIDSYLAELPAVGRSKKQGLLFLRGFRIAALPLDKITRDQVALFAQQRRNGLPELGLKPVKPPTILQDIQYIRVVIKHAFYVWNLNVSWQEIDFAIEGLERGRIVDRPTIRNRLPSSEELQSLTNHFYQAYAGRKTTAVPMHLIMWLAIYTCRRQDEICRMMLADFDREHGEWLIHDVKHPDGSRGNDKSFVISPAALQVIDELLQDNVQRCMTRLGGRPGSLVPLKATTISAQFTRACKVLDIRDLRFHDLRHEGATRLAEDGATIPQIQRTTLHDSWSSLQRYVNLRRRGDRLDFAEAIANACAPVKP

SEQ ID NO: 47 - protein, 351 aa, Unknown
  Halomonas sp. G11
MATIVKRPKRDGSFSYLARIRIARTGQPDYSESKTFPKKAMAAEWAKRRELELAAPGGVLTAKWKGVTLNDAIERYLHEFADGAGRSKRATIEQLRRFPIARVKITELSSEQIIDHAQMRRRSGVKPSTAALDITWLGIILKTAVAAWRMPVDLNEFESAKLLLRSKGLINRPASRDRRPTPEEIEQIRAYFQHSQKIRPSAIIPMEDIMDFAIASSRRQEEITRLTWDDLDTEAMTCWVRDAKHPRQKWGNHKRFKLTHEAMAIIQRQPRKRDEPRIFPYYSRSIGTRWRAATESKGIEDLRFHDLRHEATSRLFEAGYEIVEVQQFTLHESWDVLKRYTHLRPEKLQLR

SEQ ID NO: 48 - protein, 384 aa, Neisseria gonorrhoeae
MATITKRRNPSGETVYRVQVRVGKKGYPAFNESRTFSKKALAVEWGKKREAEIEAGPELLFKRGKVKMMTLSEAMRKYLNETLGAGRSKKMGLRFLMEFPIGGIGIDKLKRSDFAEHVMQRRRGIPELDIAPIAASTALQELQYIRSVLKHAFYVWGLEIGWQELDFAANGLKRSNMVAKSAIRDRLPTTEELQTLTTYFLRQWQSRKSSIPMHLIMWLAIYTSRRQDEICRLLFDDWHKNDCTRSVRDLKNPNGSTGNNKEFDILPMALPVIDELPEESVRKRMLANKGIADSLVPCNGKSVSAAWTRACKVLGIKDLRFHDLRHEAATRMAEDGFTIPQMQRVTLHDGWNSLQRYVSVRKRSTRLDFKEAMMQAQSDIKSGK

SEQ ID NO: 49 - protein, 380 aa, Unknown
  Acinetobacter sp. WCHA29
MGTISQRKLADGTIRFRAEIRISRKGLANFKESKTFSSMRLAQKWLAMREEEIEENPEILLGRSDVTNITLANAIEKYLDEVGNEYGRTKTYCLRLIQKFPIAQHIITKIKPADISDHVALRKNGYDKLDLKPIATSTLQHELLHIRGVLSHASVMWDVNVDLAGFDKATAQLRKTRQISSSGKRDRLPTTVELKKLTEYFYRKWQNPVYSYPMHLIMWFAIFSCRREAEITEMLLADHDVDNEVWKVRDLKNPKGSKGNHKEFNVLEPCQKMIELLQRKDVRKRMLKRGYDKDLLIPLSPRTIGGEFRNACKLLGIEDLRFHDLRHEGCTRLAEQGFTIPQIQQVSLHDSWGSLERYVSVKKRKKTIELAEVLPLIGED

SEQ ID NO: 50 - protein, 423 aa, Saccharomyces cerevisiae
MPQFGILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 51 - protein, 372 aa, Zygosaccharomycesfermentati
MATFSKLSERKRSTFIKYSREIRQSVQYDREAQIVKFNYHLKRPHELKDVLDKTFAPIVFEVSSTKKVESMVELAAKMDKVEGKGGHNAVAEEITKIVRADDIWTLLSGVEVTIQKRAFKRSLRAELKYVLITSFFNCSRHSDLKNADPTKFELVKNRYLNRVLRVLVCETKTRKPRYIYFFPVNKKTDPLIALHDLFSEAEPVPKSRASHQKTDQEWQMLRDSLLTNYDRFIATHAKQAVFGIKHGPKSHLGRHLMSSYLSHTNHGQWVSPFGNWSAGKDTVESNVARAKYVHIQADIPDELFAFLSQYYIQTPSGDFELIDSSEQPTTFINNLSTQEDISKSYGTWTQVVGQDVLEYVHSYAMGKLGIRK

SEQ ID NO: 52 - protein, 474 aa, Zygosaccharomyces bailii
MSEFSELVRILPLDQVAEIKRILSRGDPIPLQRLASLLTMVILTVNMSKKRKSSPIKLSTFTKYRRNVAKSLYYDMSSKTVFFEYHLKNTQDLQEGLEQAIAPYNFVVKVHKKPIDWQKQLSSVHERKAGHRSILSNNVGAEISKLAETKDSTWSFIERTMDLIEARTRQPTTRVAYRFLLQLTFMNCCRANDLKNADPSTFQIIADPHLGRILRAFVPETKTSIERFIYFFPCKGRCDPLLALDSYLLWVGPVPKTQTTDEETQYDYQLLQDTLLISYDRFIAKESKENIFKIPNGPKAHLGRHLMASYLGNNSLKSEATLYGNWSVERQEGVSKMADSRYMHTVKKSPPSYLFAFLSGYYKKSNQGEYVLAETLYNPLDYDKTLPITTNEKLICRRYGKNAKVIPKDALLYLYTYAQQKRKQLADPNEQNRLFSSESPAHPFLTPQSTGSSTPLTWTAPKTLSTGLMTPGEE

SEQ ID NO: 53 - protein, 514 aa, Unknown
  Tetrapisispora blattae CBS 6284
MPREKNSIVASGKVDAYSNSNVRELIRAFKECKTVQDYFIILIQVRFEIYEELFQELFGKDKVIIDKRIFGSLLSYYILHTFPKIKRVTYGTYRKNKAITINSLEIDYSRHKIQFKYRISGNRLIQLQTFLNEQSFFKPWKFRILSDGRKEENLFIIDKNPLKNHNEPNTNSKHIRNSETNLKFNQNVLEYLNKNGDPWDIYSQCFAMFENHSREMSCIRYKLISVLTFTNACRISDLIRLDPSSFHLKKNKYLGTIVCGHTFNTLNNIPRTVQFIPAYTRGCDMLQLLEEYLKINKNGPFEYVPMQNNKSPIQTTNDVNQKYQFFKEGVGAAYTKLMSVHPAHHLFKLKNAPKTDLGIYLMINYLNKIGLQNEGHRLGNWTKVCPIDGSELKKRNFTTTLTPCHSVRDSTRAIISGYYQISKYTNNNKKRMVRVHTLPEEPTSFTYSDNLQLHYGHWAKIVPHDVLAFLLEYSVTSKEARLALDTLPEILTPSLSMPYTSSSSSSSDDSHSYH

SEQ ID NO: 54 - protein, 423 aa, Unknown
  Saccharomyces eubayanus
MSKFDILYKTPPKVLVSQFIARFGEPSGEKLASCAAELTYLCWMITHNGAAIKRATFLSYNTIISKSLQYDVVKKTLQFKYKTQKAAILQASLQKLIPGWEFTIIPYYGQKEQSDVTDIVSNLQLQFESPEEVEKGNSHSKKMLKALLNEDESVWNIAEKILDSFEYTSRYTKTKAQYQFLFLATFVNCARFSDIKNVDPQSFKLIQNEYLGVIIQCLVTETKTGVSRHIYFFSAKGRLDSLVYLDEFLRYSEPVPKRINKTSSSSGNKQQYQLLKDNLVRSYNKALKSNAPYSILAIKNGPKSHIGRHLMTSFLSMKGLTELTNVVGNWSDKRASVVARTTYTHQVTAIPDHYFALVSGYYGYDQISKEMIPWKDETNPIEEWRHIEQLKGSTGGSTRYAAWNGIIAQEVLDYLSSYISRRI

SEQ ID NO: 55 - protein, 326 aa, Yersinia pseudotuberculosis
MEIEMNKANYDEILQDYFFSKSLRPATEWSYRKVINSFRRYIGDNLLPGEVDRLTVLNWRRHVLNKQGLSSITWNNKVAHMRAIFNHALLHDLVSFKNNPFNGVIVRPDVKRKKTLTQSEIKKIYLIMEAREREEHVGIMGKSRSALRPAWFWLTVVDTLRYTGMRQNQLLHIRLGDVNLNDGWINLRPEASKNHKEHRIPIARVLRPRLERLVATAIEKGANQVDQLFNISRIDGRKETVTENMDSPPLRSFFRRLSVECRCTISPHRFRHTIATEMMKSPDRNLKVVQTLLGHSSIAVTLEYVEGDIDSLRLALEETFERKEVF

SEQ ID NO: 56 - protein, 270 aa, Haemophilus influenzae
MQHNCNLKYPDEVSKLLILQWRKAVVGKSIIEVTWNSYVRQLKTIFKFGIENQFLPFTKNPFDGLFIREGKRKRKVYSPSDLDRLSFGIKESKYLPAILRPLWFTRALIMTFRYTAIRRSQLNKLRIRDIDLLNQVIHISPEINKNHEYHILPISHTLYPYLDNLLNELKKMKQSADAQLFNINLFSKAVKRRGKEMTADQISYLFKVISKHTGVNSSPHRFRHTAATNLMKNPENLYVVKQLLGHKDIKVTLSYIESDISSLRKHIDCL

SEQ ID NO: 57 - protein, 337 aa, Salmonella choleraesuis
METNITWQQLIDEYFFAKPLRSASEWSYTKVFKSFVHYMGPLSCPNDVTYHKVLAWRRFLLKEKKLSGRTWNNKVAHMRAIFNYGIQRGLLQYDENPFNNSVVKPDKKRKKTLTQAQIEYAYQIMEQYENQENTGLGLKYSRCALFPAWFWLTVLDTLYYTGIRQNQLLHIRLNDVDLREGQIRLITEGCKNHKEHYVPVISFLRPRLTCLVEKAQSEGLKGNDRLFNIALFTGKDPAIGDDMDSPQVRAFFRRLSKECQFAISPHRFRHTLATEMMKMPEQNLHMAQSVLGHSNMKSTLEYVENDIAVMGRALEAQFMQIKAAHARSIYSGLTKNR

SEQ ID NO: 58 - protein, 327 aa, Yersinia enterocolitica
MEMEMNQVNYDDILQDYFFSKSLRPATEWSYRKVINSFRRYIGDNLLPGEVDRQIVLNWRRHVLNKQGLSSITWNNKVAHMRAIFNHALLYDLVVLKHNPFNGVIVRPDVKRKKTLTQSEIEKIYLIMEAREREEHVGIMDKSRSALRPAWFWLTVVDILRYTGMRQNQLLHIRLGDVNLNDGWINLRSEASKNHKEHRVPIARVLRPRLERLVAAAIDKGANQADQLFNISRFDGRKESITENMDNPPLRSFFRRLSVECRCTISPHRFRHTIATEMMKSPDRNLKVVQTLLGHSSIAVTLEYVEGDIDSLRLALEETFERKAVFF

SEQ ID NO: 59 - protein, 318 aa, Unknown
  Dickeya dianthicola
MTDIGYESLLDDYFFSKSLRPATEWSYRKVTNSFIRFASDIPPCRVDRAAVLHWRRHLLTEKKVSARTWNNKVAHMRAIFNHGIKTRLLPHTENPFNNVITRPDMKRKKTLAAGQLDAIDRLMEQHLELERQGMGVNFNECALYPAWFWKTVLDTLRYTGMRQNQLLHIRLSDVNLDLGIINLRPEGSKNHREHRVPVISVLRQGLSRLIEESVAREAQPDEQLFNVYRFIGRASNDMVPMSEIPLRSFFRRLSNECRFTVSPHRFRHTLATEMMKSPDRNLQIVKNLLGHSSLTTTLEYVESNIDSIRAALEGELRC

SEQ ID NO: 60 - protein, 319 aa, Unknown
  Erwinia mallotivora
MEQRMTFEDILTDYFFSKVLRPATEWSYRKVVKTFTEFCGDDINPEHITRMDILKWRRHVLVEQKLSKRTWNNKVSHMRAIFNHAISHKLTSHEDNPFSMVVVRPDIKRKKTLTDEQIKKACLVMERKIMEEERGTHEHRANALKPAWFWMTVIDTLRYTGMRQNQLLHIRLCDVDLKNGVINLCPEGSKNHREHRVPVTDRLRPGLAVLHARSVDKGAKPEDQLFNINRFTYKKNVQGKNMDHPPLRSFFRRLSRECGCIISPHRFRHTIATDLMKRPERSLNDVQMLLGHSSLAVTLEYVEANIDNLRKNLEAAFAF

SEQ ID NO: 61 - protein, 319 aa, Unknown
  Kosakonia radicincitans
MENSITFGEIIENYFFSKTLRNATEWSYRKVLKSFLHFAGGNMMPEDVDDKLVLNWRRHVINEEGLSKITWNNKLTHMRALFNYSMAEGYVSHKKNPFNGKIARPDVKRKKTLTDIQIKKTYLLMESREIDEFTGNIETRRNALKPAWFWFTVLDTFSRTGMRQNQLLHIRLRDVDLEHSWISLCPEGSKNHKEHRVPITAMLRPRLESLYNKAVERGAGLNDQLFNVSRFDVNRKETATNMDNPPLRAFFRRLSKECGFVVSPHRFRHTIATNLMRLPDRNIKLTQDLLGHSTPAVTLQYVESDIDKVRSVLEQLDAA

SEQ ID NO: 62 - protein, 343 aa, Serratia marcescens
MKSEEKMHDEWEFLLEEYFFTKQLRSATEWSYRKVVLTFTRFIGGTITPAMVTQRDVLLWRRHLLKEKNLSVHTWNNKVAHLRAIFNLGIKKTLIQHTENPFNGTVVRSDTKKKRILTKSQLTRLYLVMQQYEQREKERKPVKGGRCALYPTWFWMTVLDTFRYTGMRNNQMIHIRLRDVNLEQGWIELRLEGSKTHREWKVPVVRQLRERIKLLIMRATERGAGQHDLLFDVKRFTSPRHAHYIYDEKNVLQSFRSFYRRLSRESGFDISSHRFRHTLATELMKSPDRNLKLVKDLLGHRNVSTTMEYIELDMEVAGKALEQELVLHTDITATRSLQSLTQA

SEQ ID NO: 63 - protein, 333 aa, Citrobacter braakii
MKEKITWTEFVEEYILEKELRTASEWSYRKVSSCFAEHLGPFVFPEDVTRRHALLWRRRVLKVEKRQETTWNNKASHMNALFNYAIKRRLFEIDENPFAETKVKAGKKKKKTMRQAQISHAYRVMEAHEEEERRLGILASRNALFPAWFWLTMMDTLYYTGMRQNQLLHLRVGDIFLDENIIRLGNKGSKNHQEHFLSVVSYLKPRLALILQKAAERGLKKNDLLFNIPVFTGKDENITEDMGSPPVRSFFRRLSRECGFTMTSHRFRHTLATEMMKLPEQNLYITRNVLGHSSMKSTLEYVERDLDAERRVLEKQFAVLKKHKVIDHCDEDG

SEQ ID NO: 64 - protein, 418 aa, Pseudomonas putida
MCAQTARLSDRQLKAVKPKDKDYVLTDGDGLQLRVRVNRSMQWNFNYRHPVTKNRINMALGSYPEVSLAQARRKAVEAREVLAQGIDPKAQRNDLAQAKLAETEHTFEKVASAWFELKKDSVTPAYAEDIWRSLTLHVFPSMKSTPISEVSAPMVIKILRPIESKGSLETVKRLSQRLNEIMTYGVNSGMIFANPLSGIRAVFKKPKKENMAALPPEELPELMLEIANASIKRTTRCLIEWQLHTMTRPAEAATTRWVDIDFERRVWTIPPERMKKSRPHSIPLSDQAMSLLEILKSHSGHREYVFPADRNPRTHANSQTANMALKRMGFQDRLVSHGMRSMASTILNEHGWDPELIEVALAHVDKDEVRSAYNRADYIERRRPMMAWWSEYILKASTGNLSASAMNVARDRNVVPIR

SEQ ID NO: 65 - protein, 395 aa, Unknown
  Yoonia vestfoldensis SKA53
MPLSDIQVRNLKPREKAYKVSDFEGLFVLVKPNGSKLWQFKYRMDGKERLLSIGVYPNISLAQARKTKDGARANVAAGIDPSEAKQQEKRQRREVNDQTFEKLGAEFFAKQRKEGKSAATLSKTEYHLQLASRDFGRKPIIEITAPMILKTLRKVEAKGHYETAHRLRSRIGSIFRYAVASGIAETDPTYALRDALIRPTRKHRAAIIDPQALGRLMNEIDVFEGQATTRIALKLLAMVAQRPGEIRHAKWSEIDFVKKVWSIPADRMKMRRDHIVPLPDQAIALLDQLRRMNGNGEYLFPSLRTWKRPMSENTLNAALRRMGYSGDEMTAHGFRASFSTLANESGLWNPDAIERALAHVEKNEVRRAYARGEHWEERVRLANWWAGYLENLQAM

SEQ ID NO: 66 - protein, 447 aa, Burkholderia cenocepacia
MAVRGFLLQTSTSDHQWKQPPIWGSFGGFAKHPLQTPPRHQHMALTDLKVRTAKPAEKQQKLYDGSGLLLLITPAGGKRWIFKYRIDGKEKSLALGTYPDISLAEARSRRDSAREKLAAGLDPSEAKKADKRAAQLAAASSFEIVAREWFETQRGGWSEVYAGKVINCLEVDVFPRLGARPIASIDAPELLAIIRTVESRGVRETAKRVLQRSRAVFQYGIMTGRCARNPAADIDAETVLKKSTGVQHMARVKVTEIPQLMRDIDEYSGDLVTRLALRFMALTFVRTKEMIQAEWPEIDVGAAEWRVPAERMKMRDPHIVPLSRQALDVLAQLREINGQQRFVFYSVQGRSHISNNTMLYALYRMGYKSRMTGHGFRGLAATTLRELGYSRDVVERQMAHAERNQVTAAYVHAEYLPERRKMMQHWADHLDELRAGAKIIPITASTP

SEQ ID NO: 67 - protein, 395 aa, Unknown
  Ahrensia sp. R2A130
MALTDARIRNLKPREKPFKTADYDGLYVLTNPNGSKLWRLKYRFMDKERLLTLGKYPSVSLADARQARDDARERLAQGQDPNDTKRQKTLAAKISHGNSFSKIAEQYMAKIIKEGRAESTLAKIDWLMDMANADLGSKPITEITSPMVLHTLKKVETKGNYETAKRLRSQIGAVFRFAIANALAENDPTFALRDALVNVKATPRAAILDKAVLGGLMRSIDGFDGQTTTRLGMELLAIVVTRPGELRHARWEEFDFDQAVWAVPAPRMKMRKPHFVPLPARALEILEELRMLNGWGQLVLPSIKSSIRPMSENTMNAALRRMGYGGDEMTSHGFRATFSTIANESGLWNPDAIEKALAHVEANKVRGAYARGQYWDERVRMANWWSGLLSDLRTQ

SEQ ID NO: 68 - protein, 398 aa, Unknown
  Hellea balneolensis
MALTDAKIRALKPKGKSYKVSDFGGLYLSVTSKGSKLWRQKYRFNGKEGTLSFGPYPEVSLKEARDQRDEAKANLKKGLNPADLKRKAAAEELGKSEYTFNKVADNFVKKLTKEGRSPATLSKLDWLLKDARKDFGHMPIATITAPIILKTLRKRETQEHYETASRMRSRIGGVFRYAVASGITDTDPTYALRDALIRPTVTHRAAIVTKDGLAELVMAIDEYRGSRQTAIALKLLMQFACRPGEIRQAKWEEFNFEECVWSIPSNRMKMRRPHKVPLTKSSLLLLEELKELTGWGEFLFPAQTSSKKPMSDNTMNQALVRMGFRKDEVTPHGFRSTFSTFANESGLWAPDVIEAYCARQDRNAVRRAYNRSLYWGERVKLANWWANILCNITTHHDD

SEQ ID NO: 69 - protein, 407 aa, Rhizobium loti
MALSDVKCRNARPASKLFKLSDGGGLQLWVQPTGSRLWRLAYRFDGKQKLLALGSYPLISLAEARQARDDAKRLLLAGMDPAHERRSRKAGSAKDTFRSIAEEYVDKLKKEGRADRTITKVKWLLDFAYPTIGDTCIREIDAATILVALRSVEVRGRYESARRLRSTIGSVFRYAIATARAGTDPTSALRDALIRPIVTPRAAITEPKALGGLLRAIDAFDGQTTSRTALKLMALLFPRPGELRGAEWEEFDFESSVWTIPETRMKMRRPHRVPLSRQAITILIRLREISGAGTLLFPSVRSTSRPISDNTLNAALRRMGYSKEEATAHGFRATASTLLNECGKWHPDAIERQLAHIEKNDVRRAYARAEHWEERVRMVQWWADYLDKIGNAKTERRPLAPKALRYE

SEQ ID NO: 70 - protein, 403 aa, Unknown
  Epibacterium mobile
MPVLSDAKVRALKPKEKPYKQADFDGLFLLVNPGGSKLWRFKYRWMGKEKLLSFGKYPDLSLKQARDQRDDARKLLAEGKDPSFERKRAQTAKEAEHRETFSRLADALLEKKRLEGKSASTLAKTEWLHGLLCADLGAYPISQISARDVLVPLRKMEAKGRNESALRMRSAAGQIFRYAIAQGLIENDPTFGLRDALTRAPVRHRSALIDPEKVGGLMRAIAGFDGQPTTRLALQLLAVTALRPGELRMAEWSEIDLDKAIWTVPAHRAKMRRPHMVPLSPEALGKLRELQELTGWGQLLFPSIRSSKRCMSENTLNAALRRMGYSGEDMTAHGFRATFSTLANESGLWSADAIERALAHVEGNEIRKAYARGTHWDERVRIAAWWAGYLQQLADNAGQHQTP

SEQ ID NO: 71 - protein, 407 aa, Unknown
  Bosea sp. BIWAKO-01
MPLTDTAIKNAKALSKVRKLSDGGGLQLWLMPTGAKLWRLAYRFDGKQRKLSIGAYPGIDLKAARAAREEAKEHLRAGRDPSEQKRLDRITKQETRATTFTSLAAELKAKKQREGKAEGTIEKFEWLLSMAEKDLGKRPVAEISAAEVLSVLRKSEKRGHLETAKRLRSVIGQVFRYAIAAGKVANDPTLALRGALAMPKPTSRAAITDPKRLGALLRAIDGYEGQNQTRAALQLMALLFQRPGELRSAEWSEFNLDEAVWLIPAARMKMRREHAVPLPRQALLTLEELREISDRSPLLFPSLRSASRPMSDVTMNAALRRLGYAKDEMTPHGFRATASTLLNECGKWSSDAIEKALAHQERNAVRRAYARGEHWQERVRMAQWWADYLDTLRNGATIIPMPAKDTG

SEQ ID NO: 72 - protein, 396 aa, Unknown
  Rhodobacter aestuarii
MPLSDVTIRNLKPRDRSYKVSDFDGLFVLVKPTGARLWQFKYRIDGKEKLLSIGRYPEIGLAQARLARDEARSMVANGRDPSAAKQERKRAELERRGVTFETQAQAFLEKTRKEGLASTTLAKNEWLLAMAIADFGAKPMSEISAQMILRCLRKVEAKGNYETAKRLRAKISAVFRYAVANGVAETDPTYALRDALVRPKAKPRAAIIDPQALGGLMRAIETYTGQRVTKIALELLALMVPRPGELRQARWEEIDLDARIWAIPAERMKMRRPHRIPLSDRAVRLLHELRELTGWTGFLLPSLVSPRRVMSENTLNTALRRMGFGADEMTSHGFRASFSTLANESGLWNPDAIERALAHIEQNDVRRAYARGEHWDERVRLAQWWADYLETLRTSA

SEQ ID NO: 73 - protein, 399 aa, Unknown
  Henriciella aquimarina
MPLTDIQLRQLKPREKDYKTADGGGLYVHVSKTGSRLWRFRYRFDGKQKLLAFGAYPAISLARARELRAEAKTLLAEGIDPAAHAKAEKAQQAALTEHTFEKIAAELVEKLRKEGKADVTLTKKQWLLDMANADFGDRPITAITAADILTTLRKVEAKGNYETAKRLRSTIGQVFRYAIATARAENDPTYGLRGALVAPKVSHMAAITDWDGFGDLIRAIWDYEGGSPSTRAALKLMALLYTRPGELRLALWDEFDLEKSTWTIPAARTKMRREHTKPLPSLAVDILKTLRAETGSNYRVFPSSIARDKPISENTLNQALRRMGFDKHEHTSHGFRATASSLLNESGLWNADAIEAELGHVGADEVRRAYHRARYWDERVRMADWWANQITKTISTARL

SEQ ID NO: 74 - protein, 346 aa, Klebsiella pneumoniae
MNRYNRNDKPDWVPPRSIKLLDQVRERVRYLHYILQTEKAYVYWAKAFVLWTARSHGGFRHPREMGQAEVEGFLTMLATEKQVAPATHRQALNALLFLYRQVLGMELPWMQQIGRPPERKRIPVVLTVQEVQTLLSHMAGTEALLAALLYGSGLRLREALGLRVKDVDFDRHAIIVRSGKGDKDRVVMLPRALVPRLRAQLIQVRAVWGQDRATGRGGVYLPHALERKYPRAGESWAWFWVFPSAKLSVDPQTGVERRHHLFEERLNRQLKKAVVQAGIAKHVSVHTLRHSFATHLLQAGTDIRTVQELLGHSDVSTTMIYTHVLKVAAGGTSSPLDALALHLSPG

SEQ ID NO: 75 - protein, 325 aa, Shigella sonnei
  misc_feature (179)..(179): Xaa can be any naturally occurring amino acid
MSNSPFLNSIRTDMRQKGYALKTEKTYLHWIKRFILFHKKRHPQTMGSEEVRLFLSSLANSRHVAINTQKIALNALAFLYNRFLQQPLGDIDYIPASKPRRLPSVISANEVQRILQVMDTRNQVIFTLLYGAGLRINECLRLRVKDFDFDNGCITVHDGKGGKSRNSLLPTRLIPAIKXLIEQARLIQQDDNLQGVGPSLPFALDHKYPSAYRQAAWMFVFPSSTLCNHPYNGKLCRHHLHDSVARKALKAAVQKAGIVSKRVTCHTFRHSFATHLLQAGRDIRTVQELLGHNDVKTTQIYTHVLGQHFAGTTSPADGLMLLINQ

SEQ ID NO: 76 - protein, 344 aa, Acinetobacter baumannii
MKTATAPLPPLRSVKVLDQLRERIRYLHYSLRTEQAYVNWVRAFIRFHGVRHPATLGSSEVEAFLSWLANERKVSVSTHRQALAALLFFYGKVLCTDLPWLQEIGRPRPSRRLPVVLTPDEVVRILGFLEGEHRLFAQLLYGTGMRISEGLQLRVKDLDFDHGTIIVREGKGSKDRALMLPESLAPSLREQLSRARAWWLKDQAEGRSGVALPDALERKYPRAGHSWPWFWVFAQHTHSTDPRSGVVRRHHMYDQTFQRAFKRAVEQAGITKPATPHTLRHSFATALLRSGYDIRTVQDLLGHSDVSTTMIYTHVLKVGGAASNGRLRKVLPASADGRQQPVVA

SEQ ID NO: 77 - protein, 337 aa, Klebsiella pneumoniae
  misc_feature (325)..(325): Xaa can be any naturally occurring amino acid
MKTATAPLPPLRSVKVLDQLRERIRYLHYSLPTEQAYVHWVRAFIRFHGVRHPATLGSSEVEAFLSWLANERKVSVSTHRQALAALLFFYGKVLCTDLPWLQEIGRPRPSRRLPVVLTPDEVVRILGFLEGEHRLFAQLLYGTGMRISEGLQLRVKDLDFDHGTIIVREGKGSKDRALMLPESLAPSLREQLSRARAWWLKDQAEGRSGVALPDALERKYPRAGHSWPWFWVFAQHTHSTDPRSGVVRRHHMYDQTFQRAFKRAVEQAGITKPATPHTLRHSFATALLRSGYDIRTVQDLLGHSDVSTTMIYTHVLKVGGAGVRXPLDALPPLTSER

SEQ ID NO: 78 - protein, 337 aa, Citrobacter freundii
MKTATAPLPPLRSVKVLDQLRERIRYLHYSLPTEQAYVHWVRAFIRFHGVRHPATLGSSEVEAFLSWLANERKVSVSTHRQALAALLFFYGKVLCTDLPWLQEIGRPRPSRRLPVVLTPDEVVRILGFLEGEHRLFAQLLYGTGMRISEGLQLRVKDLDFDHGTIIVREGKGSKDRALMLPESLAPSLREQLSRARAWWLKDQAEGRSGVALPDALERKYPRAGHSWPWFWVFAQHTHSTDPRSGVVRRHHMYDQTFQRAFKRAVEQAGITKPATPHTLHHSFATALLRSGYDIRTVQDLLGHSDVSTTMIYTHVLKVGGAGVRSPLDALPPLTSER

SEQ ID NO: 79 - protein, 357 aa, Bacteriophage HK022
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSDSMAARYRDSRGREWDKIEIDK

SEQ ID NO: 80 - protein, 356 aa, Bacteriophage HK97
MGRRRSHERRDLPPNLYIRNNGYYCYRDPRTGKEFGLGRDRRIAITEAIQANIELFSGHKHKPLTARINSDNSVTLHSWLDRYEKILASRGIKQKTLINYMSKIKAIRRGLPDAPLEDITTKEIAAMLNGYIDEGKAASAKLIRSTLSDAFREAIAEGHITTNPVAATRAAKSEVRRSRLTADEYLKIYQAAESSPCWLRLAMELAVVTGQRVGDLCEMKWSDIVDGYLYVEQSKTGVKIAIPTALHVDALGISMKETLDKCKEILGGETIIASTRREPLSSGTVSRYFMRARKASGLSFEGDPPTFHELRSLSARLYEKQISDKFAQHLLGHKSDTMASQYRDDRGREWDKIEIK

SEQ ID NO: 81 - protein, 356 aa, Coliphage lambda
MGRRRSHERRDLPPNLYIRNNGYYCYRDPRTGKEFGLGRDRRIAITEAIQANIELFSGHKHKPLTARINSDNSVTLHSWLDRYEKILASRGIKQKTLINYMSKIKAIRRGLPDAPLEDITTKEIAAMLNGYIDEGKAASAKLIRSTLSDAFREAIAEGHITTNHVAATRAAKSEVRRSRLTADEYLKIYQAAESSPCWLRLAMELAVVTGQRVGDLCEMKWSDIVDGYLYVEQSKTGVKIAIPTALHIDALGISMKETLDKCKEILGGETIIASTRREPLSSGTVSRYFMRARKASGLSFEGDPPTFHELRSLSARLYEKQISDKFAQHLLGHKSDTMASQYRDDRGREWDKIEIK

SEQ ID NO: 82 - protein, 329 aa, Unknown
  Salmonella phage ST64B
MGRKRAPGNEWMPKGVFFRPSGYYWKPGGSTENIAPADATKAEVWVAYEKKVEGRKNRITFTQLWRKFLASADYADLAPRTQKDYLAHEKYILAVFGDAEAKAIKPEHIRRYMDARGQKSRVQANHEHSSMSRVFRWSYQRGYVPGNPCVGVDKFPKPQRDRYITDEEYRAIYNNATPAVRAAMEIAYLCAARVSDVLKMNWNQILEKGIFIQQGKTGVKQIKSWTDRLRDAVEICREWGEEGPVIRTMYGERYSYKGFNEAWRKARKAAGDDLGRPLDCTFHDLKAKGISDYEGTAKDKQKYSGHKTESQVLVYDRKVKMSPTLDRKR

SEQ ID NO: 83 - protein, 367 aa, Unknown
  Pseudomonas phage phi2
MAPRPRKEGSKDLPPNLYKKTDSRSGVTYYAYRDPVSGRMFGLGKDKARAIREAIEANHTEALQPTIADRLNSEPSRPPRLFDDWLIEYEKIYAERGLAAASVRNTRMRLKRLRARFGTMDIRDIGTIDVAGYFSEMAKEGKAQMARAMRSLLRDVFMESMAAGWTDKNPVEVTKAARVKIKRERLTLETWRLIYAEAKQPWLKRAMELAVITGQRREDLAAMQFKDEQDGYLQVVQSKTGMRLRISTSIGLAVLGLDLASVIKSCRGRVLSRYMIHHHRTISRAKAGQPIMLDTISAAFADARDRAAKKHGLDFGASPPSFHEMRSLAARLHEEEGRDAQRLLGHRSAKMTDLYRDSRGAEWIDVA

SEQ ID NO: 84 - protein, 337 aa, Bacteriophage HP1
MAVRKDTKNGKWLAEVYVNGNASRKWFLTKGDALRFYNQAKEQTTSAVDSVQVLESSDLPALSFYVQEWFDLHGKTLSDGKARLAKLKNLCSNLGDPPANEFNAKIFADYRKRRLDGEFSVNKNNPPKEATVNREHAYLRAVFNELKSLRKWTTENPLDGVRLFKERETELAFLYERDIYRLLAECDNSRNPDLGLIVRICLATGARWSEAETLTQSQVMPYKITFTNTKSKKNRTVPISKELFDMLPKKRGRLFNDAYESFENAVLRAEIELPKGQLTHVLRHTFASHFMMNGGNILVLKEILGHSTIEMTMRYAHFAPSHLESAVKFNPLSNPAQ

SEQ ID NO: 85 - protein, 405 aa, Salmonella enterica
MKVSVNKRNPNSKGLQQLRLVYYYGVVEGEDGKKRAKRDYEPLELYLYENPKTQAERQHNKEMLRQAEAARSARLVESHSNKFQLEDRVKLASSFYDYYDKLTASKESGSSSNYSIWISAGKHLRSYHGRAELTFEEIDKKFLEGFRKYLLEEPLTKSQSKLAKNTASSYFNKVRAALNEAFREGIIRDNPVQRVKSVKAENTQRTYLTLDEVRAMTKAECRYDVLKRAFLFSCTTGLRWSDIQKLTWKEIEEFQDGHYRIIFKQAKLLNAGNSLVYLDLPDSAVKLMGERQDKAERVFKGLKYSSYTNVALLHWAMLAGVQKHVTFHVGRHTFAVAQLNRGVDIYSLSRLLGHSELRTTEIYADILESRRVTAMRGFPDIFEDKVQESGTCCPHCGKSVLNKTL

SEQ ID NO: 86 - protein, 337 aa, Bacteriophage P2
MAIKKLDDGRYEVDIRPTGRNGKRIRRKFDKKSEAVAFEKYTLYNHHNKEWLSKPTDKRRLSELTQIWWDLKGKHEEHGKSNLGKIEIFTKITNDPCAFQITKSLISQYCATRRSQGIKPSSINRDLTCISGMFTALIEAELFFGEHPIRGTKRLKEEKPETGYLTQEEIALLLAALDGDNKKIAILCLSTGARWGEAARLKAENIIHNRVTFVKTKTNKPRTVPISEAVAKMIADNKRGFLFPDADYPRFRRTMKAIKPDLPMGQATHALRHSFATHFMINGGSIITLQRILGHTRIEQTMVYAHFAPEYLQDAISLNPLRGGTEAESVHTVSTVE

SEQ ID NO: 87 - protein, 387 aa, Bacteriophage P22
MSLFRRGETWYASFTLPNGKRFKQSLGTKDKRQATELHDKLKAEAWRVSKLGETPDMTFEGACVRWLEEKAHKKSLDDDKSRIGFWLQHFAGMQLKDITETKIYSAIQKITNRRHEENWKLMDEACRKNGKQPPVFKPKPAAVATKATHLSFIKALLRAAEREWKMLDKAPIIKVPQPKNKRIRWLEPHEAKRLIDECQEPLKSVVEFALSTGLRRSNIINLEWQQIDMQRKVAWIHPEQSKSNHAIGVALNDTACRVLKKQIGNHHKWVFVYKESSTKPDGTKSPVVRKMRYDANTAWRAALKRAGIEDFRFHDLRHTWASWLVQAGVPISVLQEMGGWESIEMVRRYAHLAPNHLTEHARQIDSIFGTSVPNMSHSKNKEGTNNT

SEQ ID NO: 88 - protein, 441 aa, Unknown
  Salmonella phage Fels-1
MTLLDAGGIMAKPAYPTGVEKHGDKLRICFHYKGRRVRENLGVPDTPKNRKVAGELRASVCFAIKVGTFDYAAQFPDSPNLKLFGIVNKEITVAELADKWLKLKEMEISKNTMLRYESIIKISVSLLGGRVLASSVTQEDLLFFRKELMTGHHITRPGRELAPKGRSVATVNSYLGVVSGLFQFAARNGYIPQNPFNGITMLKRAKAEPDPLSREEFARLIDACHHQQIKNLWSLAVYTGMRHGELCALAWEDIDLKAGTLIVRRNYTQAKEFTLPKTQAGTDRVIHLVQPAIDALKSQASFTKLSKQHKIEVKLREYGRTKTHSCTFVFNPQITDRSGKSKAHYAAPSLNRIWESALRRAGLRHRKAYQSRHTYACWALAAGANPNFIASQMGHSNAQMVYTVYGAWMADNNQSQVDILNQQLASTAPGVPQKDNMLNFI

SEQ ID NO: 89 - protein, 345 aa, Bacteriophage K139
MSVRNLKDGSKKPWLCECYPQGREGKRVRKRFATKGEATAYENFIMREVDDKPWMGSKPDNRRLSELLETWWQVHGHTIKSGKVVYRKTALTIKELGDPIASTFTSKQYLAFRASRVSHFNKENKSLSPTYQNFQLNLLSGMFSRLIKYKQWNLPNPLDDIEPIKVNQRALAYLDKADIQPFLQRLGGFESDGRSVSIPEIVLIAKICLATGARISEALSLERSQISEFKLTFVETKGKRIRSVPISENLYKEIMLASSSSTKIFSTTYGSAHRYIKKALPDYVPEGQATHVLRHTFATHFMMNRGDILILQRILGHQKIEQTMAYAHFSPDHLIQAVQLNPLEN

SEQ ID NO: 90 - protein, 387 aa, Shigella flexneri bacteriophage V
MSLFRRGEIWYASFTLPNGKRFKQSLGTKDKRQATELHDKLKAEAWRVSKLGEIPDITFEEACVRWLEEKAHQKSLDDDKSRIGFWLQHFAGMQLRDITESKIYSAIQKMTNRRHEENWRLRAEACRKKGKPVPEYTPKPASVATKATHLSFIKALLRAAEREWKMLDKAPIIKVPQPKNKRIRWLEPHEAQRLIDECPEPLKSVVEFALATGLRRSNIINLEWQQIDMQRRVAWINPEESKSNRAIGVALNDTACRVLKKQIGNHHRWVFVYKESCTKPDGTKAPTVREMRYDANTAWKAALRRAGIDDFRFHDLRHTWASWLGQAGVPLSVLQEMGGWESIEMVRRYAHLAPNHLTEHARQIDSILNPSVPNSSQSKNKEGTNDV

SEQ ID NO: 91 - protein, 374 aa, Bacteriophage phi LC3
MATYQKRGKTWQYSISRTKQGLPRLTKGGFSTKSDAQAEAMDIESKLKKGFIVDPIKQEISEYFKDWMELYTKNAIDEMTYKGYEQTLKYLKTYMPNVLISEITASSYQRALNKFAETHAKASTKGFHTRVRASIQPLIEEGRLQKDFTTRAVVKGNGNDKAEQDKFVNFDEYKQLVDYFRNRLNPNYSSPTMLFIISITGMRASEAFGLVWDDIDFNNNTIKCRRTWNYRNKVGGFKKPKTDAGIRDIVIDDESMQLLKDFREQQKTLFESLGIKPIHDFVCYHPYRKIITLSALQNTLEHALKKLKISTPLTVHGLRHTHASVLLYHGVDIMTVSKRLGHASVAITQQTYIHIIKELENKDKDKIIELLLEL

SEQ ID NO: 92 - protein, 345 aa, Unknown
  Erwiniaceae
MAIRKLPEGGWLSELYPNGAKGKRIRKKFATKGEALAYEQHAVQLPWNEEQTDRRTLKDLITSWYSAHGITLKDGEKRQLAMLHAFECMGEPLAVDFDAQMFSRYRERRLKGDFARSSRVKEVSPRTLNLELAYFRAVFNELGRLGEWKGENPLRHIRPFRTEESEMAWLTHSQIAHLLAECRNSDQADLETVVKICLATGARWSEAEGLKKSQISKYKITYIKTKGRKNRTVPITESIYRIIPENKTGRLFADCYGAFRSALERTGIELPAGQLTHVLRHTFASHFMMNGGNLLVLQRVLGHTDIKMTMRYAHFAPDHLEEAAKLNPLAQSGDEMAIEMANVGN

SEQ ID NO: 93 - protein, 386 aa, Unknown
  Escherichia phage HK75
MSIKLRGGTWHCDFVAPDGSRVRRSLETSDKRQAQELHDRLKAEAWRVKNLGESPKKLFKEACIRWLREKSDKKSIDDDKSIISFWMLHFRETILSDITSEKIMEAVDGMENRRHRLNWEMSRDRCLRLGKPVPEYKPKLASKGTKTRHLAILRAILNMAVEWGWLDRAPKISTPRVKNGRIRWLTEEESKRLFAEIAPHFFPVVMFAITTGLRRSNVTDLEWSQVDLDKKMAWMHPDETKAGNAIGVPLNETACQILRKQQGLHKRWVFVHTKPAYRSDGTKTASVRKMRTDSNKAWKGALKRAGISNFRFHDLRHTWASWLVQSGVSLLALKEMGGWETLEMVQRYAHLSAGHLTEHASKIDAIISRNGTNTAQEENVVYLNAR

SEQ ID NO: 94 - protein, 412 aa, Unknown
  Rhodococcus phage REQ3
MPRPSLPVGAHGRISRTKLPDGRWRAACRFRDADGVTRQVVRYTPPTVDRDKTGAAAERALVDALKGRSTTGDLSADSRVSELWMAYRAQLEEKNRSQSTLQDYDRMAAKILDGLGNLRVREATTQRLDTFVREIATRQGAGTGKKAKTILSGMFRIAVRYGAVQANPVREVTDLGAGRKKRAKSMDRELLVQLLADVRGSEAPCPVVLSEAQIKRGVKTTSKAGQVPSVAQFCQAADLADLIVMFAATGARIGEVLGIRWEDVDLKKRTVAIAGKVIRVKGDGLVREDSTKTESGLRQLPLPGFAVEMLEKRLVDRTGPMVFPSKVGTLRDPDTVQRQWRQVRAALDLEWVTTHTFRKTVATILDDEGLTARQAADHLGHAQVSMTQDVYLGRGRTHSAAAAALDAAVAKR

SEQ ID NO: 95 - protein, 375 aa, Unknown
  Mycobacterium phage Bobi
MPTVRKRTRSDGTPCYLVQYRFGGRGSKQGALTFDDPKAAEAFAAAVTAHGAARALEMYGIDPSPRRTDGRSKGMTVAEWVRHHIDHLTGVEQYTLDKYEQYLANDITPHLGDIPLSKLSEDDIARWVKVMETTGGRDGNGHAPKTLRNKYGFLSGALNAAVPRYLSTNPASGRRLPRGNAEDDDEIRMLTHAEFDRLRDAVTPHWKLMVQFMVSTGLRWGEVSALQPKHVDLETSTIRVRQAWKYSSAGYVLGPPKTKRSRRTVDVPARLLERLDLSNEFVFVNTDGGPVRYPGFLRRVWNPAVEKAGLVPRPTPHDLRHTYASWQLTGGTPVTIVSRQLGHESIQITVDTYTDVDRTSSRVAAEFMDGLLGDF

SEQ ID NO: 96 - protein, 365 aa, Unknown
  Mycobacterium phage Validus
MASIRTRSRKDGSTYTQVRYRLNGEETSTSFDDVGHAVEFKRMVDQLGAAKALEVIETTDAASQHYTLGEWLDHYLRHKTGVEKSTLYDYRKMVEKDIAPALGAIPLAALTAEDVAKWVQGLAEAGLAGKTISNKHGFLSSALNVAVTRGHIAANPATAGAGLIEVPRTERAEMVFLSREQYAKLHDNMPLRWQPLVEFLVASGARWGEVTALRPSDVNRADGTVRISRAWKRTYASGGYALGAPKTERSRRTINVDASVLDKLDYSHEWLFVNGRGAPVRGHNFHENHWQPAIKRAGLDVKPRIHDLRHTCASWLIAAGVPLPAIQQHLGHESIKVTIGVYGHLDRSHGKTVAAAIAAQLDPGR

SEQ ID NO: 97 - protein, 366 aa, Unknown
  Mycobacterium phage ZoeJ
MASIRSVSRKDGTTFTQVRYRLNGKQTSTSFDDGAHAVEFKRMVEQLGAAKALEVLETTDAASRNFTLAGWLKHYLDHKTGVEKSTIYDYRKMVEKDITPVLGAIPLAALTAEDVAKWVQGLADKGLAGKTIANKHGFLSSALNVAASAGHIKANPAVGGAGLVAVPRTERAEMVFLTADQYAKLHDNMPLRWQPLVEFLVASGARWGEVTALRPSDVNRAEGTVRISRAWKRTYARGGYELGAPKTNKSRRTINVDTAVLDRLDYSGEWLFTNVRGGPVRGHNFHENHWQPALKKAGLDGLDVKPRIHDLRHTCASWLIAAGVPLPAIQQHLGHESIQVTIGVYGHLDRSSGRTVAAAIAAALGR

SEQ ID NO: 98 - protein, 407 aa, Unknown
  Paenibacillus phage HB10c2
MKGHFYKPNCKCPGKKTKKCSCGATWSYIIDVGINPNTGKRKQKKKGGFKTKTEAQEAAALLVAELSQGTYVEEKNNTFEEYAKEWLSEYQATGTVKISTVRIRKKGIKLLLPYLAKLRISIITAKQYQHALLDLHDKGYSNNTIVSAHQTGRMIFQRAIELKIIKNDPTSSAVIPKRQRTIEDLETEKEIPKYMEKEELALFLQTAKEKGLDRDYAIFLTLAYTGMRVGELCALKWSDIDFSEQTVSITKTYYNPNNNIKNYTLLTPKTKSSKRVIIVDKKVLDELEQLQAEQKRIKMFFRKTYHDKNFVFSQQGEENAGFPTYPKLVALRMTRLLKLAGLNTKLTPHSLRHTHTSLLAEARVSLEQIMQRLGHRSDETTKNIYLHVTKPKKKEASQKFAELMSSF

SEQ ID NO: 99 - protein, 359 aa, Unknown
  Gordonia phage Lucky10
MASIHTRTLADGTDSYRVSWRHNGRQRRLSFENIQAATTHKLNLEKFGHDRAMQILGVIETHRDETTLTQTLEHHINSLTGVEPGTIRRYHSYLRNDFADIGQLPVSGISETVIASWITELAKKNSGKTIANKHGLLSAALARAVREGRLTANPCDHTRLPRKDPVDDPVFLDRDQFDELAAAMPEHWRPLATWLVMTGMRFSEATALTVGDITPTSTGGVVRISKAWKWTGTTEKRLSYPKSRAGRRTINVPAQAIQLLDLDRPKTRLLFTNRNDDRVTYSRFYDGGWKPAMQKTAWHASPHDLRHTCASWMIAAGVPLPVIQAHLGHESITVTIGVYGHLDRSSHESAAAAIGQMFG

SEQ ID NO: 100 - protein, 224 aa, Unknown
  Natrialba phage PhiCh1
MSKERHAHEDALNETEFQKLLDGAHLLTPPANLEATFVITMSGKLGMRIGEIAHMKRTWVKPDQGLIEVPSHEPCEKGRDGGLCGYCRRQANRTYQNDPENRDLDELLKSYWEPKTEAAERAVPYEFDEDVEDVVSSFFEYYYEVPLSVNTCRRRVKDAAEASDLNRRVYPHALRATAASTHAYEGLNIASMKAMMGWAKLSTAEKYIRISGGRTKRALLEIYG

SEQ ID NO: 101 - protein, 216 aa, Unknown
  Halalkalicoccus jeotgali
MSEREFQLLLEGAASLRDPYAQQARFVILVAGRLGMRAGEIAHMDRSWIDWRNQMIVVPRHDPCTKARGEAGPCGYCKRLAEQAADHNPELSYEAALARAWTPKTDSAARSIPFDFDPRTDLVIERFFERYEKFPHSKQAVNRRVNKAAEVTDELDEDSIYPHCLRATAATYHASRGLSALPLQSMLGWSDLSTSQKYVRRSGEATARALRTVHRQ

SEQ ID NO: 102 - protein, 223 aa, Unknown
  Halobellus rufus
MVATRERALSEREFELLLEGAGRIGDTQRRLETRAAILLGGRLGLRPGETTHLSKSWVDLERQMIQIPPQENCTKGRDGGICGYCRQAVKQRLDHNPNTDFQSFADRYWLPKTEAASRTVPYHFSYRVRVAVELLLNEHSGWPYSFSTLQRRLETALERSPELSNDATSLHGLRATAASYHAGRGLDLPALRAMFGWEDITTARQYLNVDGAMTRRALDSIHQ

SEQ ID NO: 103 - protein, 222 aa, Unknown
  Haloferax sp. ATB1
MAPTREKSLSEREFELLLEGAGRIDEPVQRLESRAAILIGGRLGLRPGETTHLSSSWIDHERQMIRIPEHHACTKGRDGGLCGYCRQAIEQRLRHDPDSRFEDFADLYWLPKTDAAARTVPFHFSYRVRVAIDLLITEHGGWPYSFSTLQRRLNTALDLAPRLSRNATSLHGLRATAASYHASRGLELAALRAMFGWEDIATARQYLNVDGAMTRRALNNIH

SEQ ID NO: 104 - protein, 233 aa, Unknown
  Halovirus HCTV-5
MRKEIRENRKGRYTREDALNDREFQLLLEGAREMEHYYSQQARFIILVAGRLGMRKGEITHIQEKWVDWRKDMIEIPRFEPCDKGKNGGACGYCKQQAKQAVEYNEEADIEEEIRCKWEPKTEAAARKIPFGFDPRTSLILERFFDRYDEFCWSAQAITRRVKKAAKLAKELDEEEIYPHCLRATAATYHASRGLEMVPLQAMFGWAQPSTAMNYIQNSGENTARALHMVHSQ

SEQ ID NO: 105 - protein, 439 aa, Unknown
  Sulfolobus sp. NOB8H2
MAEVGNHLGKIGNHLNPEVETNIMPILDIDKLTNEQKIRLFTYVTEEKGITYEQLGISKATGWRYKKGLREIPKEIMEKALQFLAPDEIARVVYGKKIEKADINDLLKVINTAVEDLQFRSLLFMMLNRFLGEYVKQNTNSYAVTEEDLKLFEKILEQKSKATKEERLRHIKYAMKDLGFSLSPESLKEYIVELAAEEGPNVARHRANTLKLFIKEVVMSRNPILGQILYNSFKVPKVDYKYSPPPISLDLLKKIFQSIDHLGAKTFFLILAETGLRVGEVYSLTLEQVDLENGIIKLMKSSATKRAYISFLHKETIEWIKKNYLPFREDFISKYEKAVQQIGGDVEKWRMKFFPFQLADLRAEVKEGMRKVGKEFRLYDLRSFFASYMAKSGVSPFIINVLQGRMAPGQFKILQQHYFVISDIELKKIYEEKAPKLLS

SEQ ID NO: 106 - protein, 413 aa, Unknown
  Sulfurisphaera tokodaii
MIVDVSSLSEEQKIKIVETVLQKGISYKELGIDRVTWWRYKNKKRKIPDEVVQKAAEYLTPDELVQLTYSIDISKIGINEAIGVIVKATKDPEFREFFLSLLQRNLGEFIKAASYSYPITQEDLQMFKKLIENKAKNTFEDYWRYINRIAKDNNYVISPDKIKDYILEQFDESPHRARQMATVLKLFIKEIVRSKDPILAQILYHSFSIPRPKTKYKPAVLSLDLLKKVFSEIQELGAKTYFLIAAETGLRTGELFYLSVNQVDLQHRIIKLFKENETKRAYIAFLHRETAKWIEENYLPYRENYIRRHWGGVKAIGQDIEKWKMKFFPMNEDKMRAEIKAAMQRGGKVFRLYDLRAFWASYMIKQGVSPMIVNILQGRAAPNQFRILQEHYLPFSEEELREIYEKYAPKLLT

SEQ ID NO: 107 - protein, 419 aa, Unknown
  Acidianus hospitalis
MLINVSKLDEQQRKRIIKKLVEKLGLSQAAKMLGVGRSTLYRYVNSDRNIPLDIVRKAAEMLAQDELSDAIYGLKVVEVDATTALSVVVKAMKDEKFRNFFVSILYQYLGDYLKSASSTYIVTEEDVKKFEKLLQGKSKSTIDMRMRYLRIALTKLGYELSPDSIRDLIAELSEDSSNIARHTANSLKLFIKTVVKEKNLQLAQLLYNSFKVPKSKYKYKPQPLTLETLRRIFDNIDHLGAKAFFLLLSESGLRVGEVYSLKVDQLDLENRIIKVMKESETKRAYISFIHTETRKWLQEVYFPYREEFVRTYEFAVKQIGADVEAWKQKLFPFQLADLRSSIKEGMRKVLGKEFRLYDLRSFFASYLIKNGVSPMIVNILQGRAPPAQFQILQNHYFVMSEIELQKVFDEKGPKLLSPK

SEQ ID NO: 108 - protein, 433 aa, Sulfolobus islandicus
MRHSKLIYINYVDGYLLIMDTTKLDDDKKLKILEKAIEKFGKAYIAQKCGVSRQTIYRYLKREIQSIPDEFIQCVSNFLSIEELGDIVYGLRTVEVDENIALSVIVKMKRDPNFRAFFLSLMKQFLGEYIQDASTSYVITKNDVDRFLNYIKSKSNTTYKTFKNYFVKTIAELNYTLTPEAVKDYITKEMTISKGRASHISKILKLFIKEIIIPKNSSLGRELYNSFKTIKVEKEYSPESLTLEDLKRVFTTIEHIGAKAFFLLLAETGLRINEILKLNIDQIDLEKRIIYVNKISASKRAYITFLHENTAKWLKETYLPYREEFINKYEKKLRNININVEAWKNRLFPINEYNMRKEIKEAMKKVLSREFRLYDLRSFFASYMIKQGVSPMIVNLLQGRAPPQQFQILQNHYFVVSDIELQQYYDKYAPRLL

SEQ ID NO: 109 - protein, 385 aa, Unknown
  Vulcanisaeta distributa
MIRSGRRRVGDGLLCSMLRLLTPEELQSLLRGWVPERRASLSDALRVIITAREDPTFREQFLALLSRYLGDYVQSLGRAWHVTQEDIEAFIKAKRLKGVGEKTLNDELRYIRRALEELDWVLTPEGITEFLGGLAEEESPYVVRHVTVSLKSLIKTVLKPRDPGLFAVLYNSFTTIKPRNHNKTKLPTLEELRQVLSKIESIEAKTYFIILAETGLRPSEPFLVSMDDVDLEHGMLRIGKITETKRTFIAFLQPKTLEFIKAQYMPRRDWLVRNRLEAIKADYLGVKPSVEDWARKFMPFDRDRLRREIKEAARQVLGRDFELYELRKFFATWMISRGVPESIVNTLQGRAPPSEHRILIEHYWSPRHEELRNWYLRHAPCLLCH

SEQ ID NO: 110 - protein, 426 aa, Unknown
  Caldivirga sp. MU80
MDPDLIRVEAIPQDVRRKVLEYVTGVKGIGPSDLGYNKTYMYRVRHGMVPISDGLFKALLRFIDIDEYARLVGSAPPLVEATPDDIVRVVKKALVDKSFRNLLFDMLRQAFGDEFREYRASWTVKEADIEEFVRAKRLKGLSGRTIRDEVRYIRLALSELNWVLEPEGIREYIAGLAEEGEYNIARHVSVGLKSILKTVLKPRDPALFRLLYDSFTVYKHKASTHVKLPTLEQLRLIWARLPSVEARFYFTVLAECGLRPSEPFLASIDDLDLEHGVIRIGKVTETKRSFVAFLRPEFADWVRESYLPAREALIKAKLDIVRADYLGVNANAEDWARRLIPFDRGRLRREIKEAAKQVLGRELELYELRKFFATWMISQGVPESIVNTLQGRAPPSEFRILVEHYWSPRHEELRQWYLRYAPRVCC

SEQ ID NO: 111 - protein, 431 aa, Unknown
  Vulcanisaeta sp. EB80
MKPMVDCELINIEKIGNEERVRIINYVMEKKGVKARDLGVTLNLISMIRSGKRRVTEDLLCRALKFLSNEELAKLLGQIPELEPASISDLVRVVARARADPEYRDLLLSYLDRYLGDYVRAMGNKWVVTEQDIEEFIKAKRLEGVTEKTLRDYTHYLREMLAELNWNLTPDGIREYLSGLAEEGEEHVLHHLTTALKSLLKTILEPRDPFLFGLLYHAFKTYKAKSNNRIKLPTIDQLRQIWQQLPTIETRFYFALLAETGLRPGEPFLLSIDDLDLEHGMLRIGKVTETKRAFVAFLRPEFLEWVKTNYLPHREAWIVRMAKLWESSNLFTQEVIEKAKRKLIPFDQSRLRREIKDTARQVLGREFELYELRKFFATHMISQGVPESIVNTLQGRAPPSEFRVLVEHYWSPRHEELRGWYLKYAPRVCCD

SEQ ID NO: 112 - protein, 419 aa, Sulfolobussolfataricus
MLTDVTKLDDEQRRRILKKLVEKLGLAQTAKLLEIGRSTLYRYVNTNQNIPLEIVRKAADMLTPDELSDVIYGLKVVEVDATTALSVVIKAMKDEKFRNFFVSVLYQYLGEYLKNTSSTYIVTGEDVKRFEKSLQGKTKSTIDMRMRYLIPALIRLGYELSPDGIRDLLAELSEESSNIARHTANSLKLFIKAVIREKNLQLAQLLYNSFKVPKSRYKYRPQPLSLETIRDIFDNISHLGARAFFLLLAESGLRVGEVYSLKLDQLDLENRVIKVMKETETKRAYVSFIHIETRKWLQEIYFPYREEFIRTYEHAVKQIGADVEVWKQKLFPFQLADLRASIKEGMRKVLGKEFRLYDLRSFFASYMIKNGVSPMIVNLLQGRAPPTQFQILQNHYFVMSEIELQRIFDEKGPKLLSLK

SEQ ID NO: 113 - protein, 419 aa, Sulfolobus islandicus
MLIDVTKLDEEQRKRILKKLIDKLGLTLAAKMLGVGRSTLYRYVNTNQSIPLEVVKKATEMLAPDELSDAIYGLKVVEVDATTALSVVIKAIKDEKFRNFFVSILYQYLGDYLKSASSTYIVTEEDVKKFEKSLQGKSKSTIDMRIRYLRMALIRLSYELSPDGIRDLLAELSEESSNIARHTANSLKLFIKTVVKEKNLQLAQLLYNSFKVPKSKYKYKPQPLSVDTLRKIFDSIDHLGAKAFFLLLAESGLRVGEVYSLKMDQLDLENRIIKVMKESETKRAYISFVHKETKEWLQGVYFPYREEFIRTYEHVVKQIGADVEAWKQKLFPFQLADLRASIKEGMKKVLGKEFRLYDLRSFFASYLIKNGVSPMIVNILQGRAPPAQFQILQNHYFVMSEIELQKIFDEKGPKLLSPK

SEQ ID NO: 114 - protein, 291 aa, Archaeoglobus veneficus
MRGLYKERAAEAFNEAVLDYDKYKEEFKEWLFKEVSKETAEQYLRDLEQTIAGKKINDPHELYNIYKDYPQRHHRKAIRTFMRFLIKSGIRKKSELMDFQAVIDIPGTQPRPPEEAFTTDEKIIEALNSPKVKKDERRQILIRLLAYTGLRLREALELLRTFDKNKLEFHGNYARYPTYELKSKAGTKRTYYAYMPADFARQLKRIDIKETTVKGAKLADRIILPEQLRKWHTNFLKRKIKEKKLQLGVTAETLINFIQGRVGKAVIDRYYLDLVEDADELYTKIADEFPF

SEQ ID NO: 115 - protein, 287 aa, Unknown
  Pyrococcus sp. NA2
MVGPRGFEPRTSTLSEKLNDLWSFYKIQFSEWLSGQITEVVRKDYIKALDKFFDRHEIVTYQDLERALKFENYTDRLVKGLRKFVTFLEEEHILDFRRADDLRRIIKLRRETRIRDVFISDEELRIAYEKVKQKELVKVVLFELLVFSGIRLSHAVQLLNSFDESKLFRINDKIARYPLFAISRGKKRGFWAYAPVELFEKIMSIGRQNINYKTAQDWVTYGKVSANTIRKWHYTFMIRQGVPAEIADFIQGRASRTVGPTHYLNKTILADEWYSVIVDELKKVLEG

SEQ ID NO: 116 - protein, 292 aa, Unknown
  Thermococcus kodakarensis
MAKKYIPLLDKYLWGKKANTPEELRKIIESIPPTKKGNPNRHAYLAIRSYINFLVDTGRIRKSEAIDFKAVIPNIKTNARAESAKVITSEDIREMFSQLKGKNETILRARKLYLKLLAFTGLRGDEVRELMNQFDPRVVEETFKAFGLPEEWRKKIAVYDMERVKLPTRRHGTKRGYVAVFPAELVRELEWFASTGYKLTADNSDKHKLFRDYTKVKDLALLRKFWQNFMNDNVMSTVPNPPADAFHLIEFLQGRAPKTVGGRNYRWNVRNAVRIYYYMVDRLKEELGILEL

SEQ ID NO: 117 - protein, 286 aa, Unknown
  Palaeococcus ferrophilus
MNPRPADYKSVIALKTLNEVWNHEKKAFLEWLSLKIGRERTVKDYYNALKVMFKDYEVRPTKKSIKNAIDALGNKKRYVYGLRNFLKYLTEKELINEDFSKMLQGAAKAKKSGVREVHLNDHEITEAWQHVKNRREEAQMLFKAMVFSGIRLAQLIRMFKTYDPARLQFPLEGIARYPIKDISEGKKKGFWAYFPADLVPELRRFSAKETTAWKWVRYGRVSANSIRKWHYTFLIRKGVPADLADFIQGREAETVGARHYLNKTLLADEWYSTVVDDLKKVLEGEK

SEQ ID NO: 118 - protein, 247 aa, Unknown
  Thermococcus kodakarensis
MKDYISALERFFGRHTIRDIKGLKVSLQQENYNEKIVKGLRNFVNFLLDEGLINEGTAALFKKPLTFKRGTPRQVFISNEELREAYIELTKHYGKEAEVLFKLLAFTGLRLKHIVKMLNTYDPQKLVIVNEKVARYPMAEHGKGTKRAFWAYMPADFARSLERMSITYFQAQPRTTYKRVSASTVRKWFSTFLAQRKVSMEVIDFIQGRAPRSVLERHYLNLTVLADEAYAKVVDDLRKVLEGQTHD

SEQ ID NO: 119 - protein, 327 aa, Unknown
  Geoglobus acetivorans
MRSSAARQFTSSISEIESNNGLIRYPEEAKGSKLHQKYNGYNERIKFEDIDYEDFELFWTAERKMKTSKGRVKRLYNVLRKVLSGKVINEESLREGFHKTTNKKDYVNAVRVLLEYLKVRKLMPREVVQEILEQPFLTPIRSKRRGIYLKDEEIRQAYEWLKEKWKDKDTELLFKLLVFSGIRLDHALDLLYNFDPRKLEFKGRVARYPLTNISNEIKSGEYAFMPAEFARKLKKIKKKLNYQTWENRINVKRWRGDEKYKKSRVDANAIRKWFGNFCLSHDVSESATEYFMGHAIKGMGGKAYFDLRDKLSWREYEKIVDKFPIPP

SEQ ID NO: 120 - protein, 318 aa, Unknown
  Thermococcus prieurii virus 1
MNEMGINKSQFFNDTARWVFLGEEMPEIIVKLEWCGGRDLNPGHRLGRSLSLNEMWVAYRAEFEKALLAEVAETTAKDYLSALNRFFGAHKIKTTEDLRNSYLKEGQKRNLGKGLRKFFTFLYQHDAISFELYQKLKNIIKLKPTKASGKFITTGELLEAYDYFRKHGRPEELLLFRILAYSGIRLRHAVQLLNSFSRDKLIYHENFAKYPLFKHEGTKVVYYAYMPRELAEELFQSGYTEDMARKYLRYGKVSASTIRKWFSTFLVSKGVPPAAVNYIQGRKPKNVLDAYYVQLEKLADEAYSRVLPDLKKVLEDGE

SEQ ID NO: 121 - protein, 455 aa, Unknown
  Thermococcus nautili
MVKSGGVYVHSQATGEEQAGARKRRRPRRLSPRLYITLPPEIYRKAKERWDNVSRIIASLLEVALAEDLTVEEVVTAVTLLRSGALVVNSPSSAGVAEPGQRRWTQDALFSPNEGLSRQNDNKEEPSADNVFTGKALIDSTAKIHYGRDRQKYIEWVKRRTPSMADKYISLLDKYLWGKKANTPEDLRRIVEAIPPTRGGFPNRHAYMALRSYINFLVDTGKLRKSEAIDFKAVIPNVKTNARAESAKVITVEDIREMFNQLKGKNETILRARKLYLKLLAFTGLRGDEVRELMNQFDPRVIDETFKAFGLPEEYKEKIAVYDMERVKIKTRRSQTKRGYVAVFPAELVPELEWFRSTGYKLTADNSDKHKLFRDSKEVKDLALLRKFWQNFMNDNVMSTVPNPPADTWHLIEFLQGRAPKNVGGRNYRWNVKNAVRIYYYMVDKLKEELGILEL

SEQ ID NO: 122 - protein, 384 aa, Shigella sonnei
MPSPRIRKMSLSRALDKYLKTVSVHKKGHQQEFYRSNVIKRYPIALRNMDEITTVDIATYRDVRLAEINPRTGKPITGNTVRLELALLSSLFNIARVEWGTCRTNPVELVRKPKVSSGRDRRLTSSEERRLSRYFREKNLMLYVIFHLALETAMRQGEILALRWEHIDLRHGVAHLPETKNGHSRDVPLSRRARNFLQMMPVNLHGNVFDYTASGFKNAWRIATQRLRIEDLHFHDLRHEAISRFFELGSLNVMEIAAISGHRSMNMLKRYTHLRAWQLVSKLDARRRQTQKVAAWFVPYPAHITTINEENGQKAHRIEIGDFDNLHVTATTKEEAVHRASEVLLRTLAIAAQKGERVPSPGALPVNDPDYIMICPLNPGSTPL

SEQ ID NO: 123 - protein, 384 aa, Salmonella enterica
MPSPRIRKMSLSRALDKYLKTVSVHKKGHQQEFYRSNVIKRYPIALRNMDEITTVDIATYRDVRLAEINPRTGKPITGNTVRLELALLSSLFNIARVEWGTCRTNPVELVRKPKVSSGRDRRLTSSEERRLSRYFREKNLMLYVIFHLALETAMRQGEILALRWEHIDLRHGVAHLPETKNGHSRDVPLSRRARNFLQMMPVNLHGNVFDYTASGFKNAWRIATQRLRIEDLHFHDLRHEAISRFFELGSLNVMEIAAISGHRSMNMLKRYTHLRAWQLVSKLDARRRQTQKVAAWFVPYPAHITTIDEENGQKAHRIEIGDFDNLHVTATTKEEAVHRASEVLLRTLAIAAQKGERVPSPGALPVNDPDYIMICPLNPGSTPL

SEQ ID NO: 124 - protein, 374 aa, Escherichia coli
MFRKIKIRKMTLNRALDKYLKTVSIHKKGHLQEFYRVNVIKRHPMAERYMDEITTVDIATYRDQRLAQINPRTGRQITGNTVRLELALLSSLFNIASVEWGTCRMNPVELVRKPKISSGRDRRLTSGEERRLSRYFRDKNQQLYVIFHLALETAMRQGEILTLRWEHLDLQHGVAHLPETKNGLPRDVPLSRKARNYLQILPQQINGNVFSYTSSGFKSAWRTALLDLKIENLHFHDLRHEAISRFFELGTLNVMEVAAISGHRSLNMLKRYTHLRAYQLVSKLDTKRKQTCKIAPYFVPYPATVGNRNGLFIVTLHDFDLETRAETRELAISHASVLLLRTLAQAAQRGERVPTPGELPANIDARVMICPLTS

SEQ ID NO: 125 - protein, 385 aa, Escherichia coli
MPSPRFRIRKMTLSRALDKYLKTVSVHKKGHLQEFYRANVIRRYPIAQRFMDEITTVDIAAYRDMRLAEINPRTGKAITGNTVRLELALLSSMYNIARVEWGTCRDNPVELVRKPRVSPGRERRLTSSEERRLSRYFRERNMSLYVAFHLALETAMRQGEILSLRWEHIDLRHGVAHLPETKNGHSRDVPLSRRARNFLQMLPVALHGGVFSYTSSGFKSAWRIATQTLRIEDLHFHDLRHEAISRFFELGSLNVMEIAAISGHRSMNMLKRYTHLRAWQLVSKLDARRRQTQKVAAWFVPYPGHITTDDGQTVRIDICDFDDLSVTAATREEALSRASEVLLRTLAIAAQKGERVPAPGALPVNDPAFVMVCPLNPQGALTAQV

SEQ ID NO: 126 - protein, 383 aa, Salmonella enterica
MSRPQRIKKMSLSKALDKYYATVSVHKRGHQQEFYRVRVIQRHPLAEKMMDEITTVDIASYRDDRLSQVNTRTGRCISGNTVRLELALLSSLYNLASVEWGTCRTNPVEMVRKPKISGGRDRRLTSQEERRLSRYFQEQNPALHAIFHLAIETAMRQGEILSLRWEHIDLQHGVAHLPMTKNGSSRDVPLSRKARHLLQGMTVALSGNVFHYSSSGFKSAWRVALQRLNIVDLHFHDLRHEAISRLFELGTLNVMEVAAISGHRSLNMLKRYTHLRAYQLVSKLDARRRQTQKIAPYFVPYPACIESINEGSDGCCGFRVHLPDFDNLSVSAASRESALEAAGVLLLRTLAKAAQRGERVPRPGDLPEGKHERVMIHPLLSAA

SEQ ID NO: 127 - protein, 376 aa, Salmonella enterica
MSQPSRIRKMTLSAALTKYYDTVSVHKRGYQQEFWRVSVIKRHPVVQKMMDEVTTVDIAAYRDDRLSQESPRTGKPISGNTVRLELALLSALYNLAKVEWGTCRTNPVEMVRKPKPSPGRDRRLTSSEERRLSRYFQARNAELYTIFHLALETGMRQGEILSLRWEHIDLQHGVAHLPVTKNGSTRDVPLSRRARNLLHELPVQLSGAVFHYKSTGFKSAWRVALQSLKIEDLHFHDLRHEAISRLFELGTLNVMEVAAISGHKSLNMLKRYTHLRAYQLVSKLDTRRRQSQKIATYFVPYPAVLEEAGDGFRVHLHDFEGMSVSGDTPESAMDAASVVLLRTLAIAAQRGERVPRPGDLPVHTGVMIDPLPGMRQ

SEQ ID NO: 128 - protein, 379 aa, Salmonella enterica
MLPSVRVKKISLFRALDRYLDTVSVHKRGYQQEFWRVSVIKRHPVAQKMMDEVTSVDIASYRDERLSQVNTRTGKPISGNTVRLELALMSALYNLAKVEWGTCRTNPVEIVRKPKPSSGRDRRLTSSEERRLSKYFQVRNAELYTIFHLALETGMRQGEILSLQWEHIDLQHGVAHLPVTKNGSVRDVPLSRRARNLLHELPVQLSGTVFHYKSTGFKSAWRVALQKLKIENLHFHDLRHEAISRLFELGTLNVMEVAAISGHKSLNMLKRYTHLRAYQLVSKLDTRRRQSQKIATYFVPYPAILEEAGDGFRVHLHDFEGMSVSGDTRESAMDTASVVLLRALATAAQRGERVPRPGDLPLNAGVMINPLAGSVPVCV

SEQ ID NO: 129 - protein, 379 aa, Citrobacter braakii
MAQPVRIKKMSLSAALTKYYDTVSVHKRGHQQEFWRVSVIKRHPVAQKMMDEVTTVDIAAYRDDRLAQVNPRTGKPISGNTVRLELALLSALYNLAKVEWGTCRANPVEAVRKPKPSPGRDRRLTSSEERRLSRYFQARNAELYTIFHLALETSMRQGEMLALRWEHIDLQHGVAHLPVTKNGSPRDVPLSRRARSLLQQLSVQISGPVFHYKSSGFKSAWRAALQRLKIENLHFHDLRHEAISRLFELGTLNVMEVAAISGHKSLNMLKRYTHLRAYQLVSKLDVRRRQSQKIATYFVPYPAEMEDTADGFRVHLHDFEGLSVSGHTREAAMDAASVMLLRRLATAAQHGERVPRPGDLPLHAGVMINPLAGAAPVFV

SEQ ID NO: 130 - protein, 374 aa, Unknown
  Enterobacteriaceae
MFRKIKIRKMTLNRALDKYLKTVSIHKKGHLQEFYRVNVIKRHPIAERYMDDITTVDIANYRDQRLAQINPRTGRQITGNTVRLELALLSSLFNIARVEWGTCRMNPVELVRKPKISSGRDRRLTSGEERRLSRYFRDKNQQLYVIFHLALETAMRQGEILTLRWEHLDLQHGVAHLPETKNGLPRDVPLSRKARNYLQILPQQINGNVFSYTSSGFKSAWRTALLDLKIENLHFHDLRHEAISRFFELGTLNVIEVAAISGHRSLNMLKRYTHLRAYQLVSKLDARRKQTSKISPYFVPYPATVRCRNGLFVVTLHDFDLETRAETRELAISHASVLLLRTLAQAAQRGERVPTPGELPANIDERVMICPLTN

SEQ ID NO: 131 - protein, 330 aa, Haloarcula marismortui
MYLKARQDELTESTIQSQEYRLEAFEQFCREEGIENLNDLSGRDLYAYRVWRREGNGKGRDEIEPITLRGQLATVRSFLRFAAEVDAVPEDLRTKVPLPTISNAGEVSASTLDPERADVILDYLQMYKYASRVHVIALLLWHTGARMGAIRGLDIDDCELEQDNPGIQFVHRPQTDTPLKNGEKGQRWNAISDHVANVLQDYIDGPREPVFDEHGRRPLVTTPQGRASTSTFRTTMYRVTRPCWRGAECPHDRDPEECEATSNRKASTCPSARSPHDVRSGRVTAYRREDVPRRVVSDRLNASDQILDKHYDRRGEREKSEQRRDYLPEV

SEQ ID NO: 132 - protein, 351 aa, Unknown
  Halorhabdus utahensis DSM 12940
MRLVEMRRWPGVSEELSPLSPEEGIDRFLRHREPSVRESTMRNARTRLRFFREWCEEREIENLNTLTGRDLADFVAWRRGDVKALTLQKQLSTIRTALRFWADVEAVQEGLAEKLHAPELPDGAESRDVALDADRAADILEYLRELHYASRDHVVMEILWRTAMRRGALRSIDVDDLRPDDHAIVLRHRIDEGTKLKNGESGERWVYLGPSTYQVIDDYLDNPDRYDVTDDHGREPLLTTPYGRPIGDTIYSWVNRLTQPCRIGGCPHDRDPSDPSTCDALGSDGSPSRCPSARSPHGIRRGSITHHLNTDVSPEIVSERCDVTLDVLYEHYDVRTDQEKMAVRKRQLSEF

SEQ ID NO: 133 - protein, 351 aa, Unknown
  Halomicrobium mukohataei DSM 12286
MPDPDLEPISPVEAVEMYHDAMVDELAESTRKSNKHRLRAFIQFCDEEEIENLNDLTGRDLYKYRIWRREGNGDGREPIKKVTLKGQLATLRSFLKFAGEIDSVKPDLYEQLSLPAMKGGEDVSESTLDPERALDILEYLEKSQPGSRDHIIIALLWETGGRTGAIRGLDLQDLDLDGDHPRFSGPAVHFVHRPETGTPLKNQKSGTRWNRISEKTAAFIEDYIEFHRPDVTDDHGRDPLLTSEYGRVAGNTYRRTLYRVTRPCWRGEECPHDRDLDECEATHLDHASKCPSARSPHDVRSGRVTYYRREDVPRKIVQERLNASEDILDRHYDRRSNREQAEQRSDFLPDV

SEQ ID NO: 134 - protein, 348 aa, Haloferax volcanii
MSELESLEPARAVRMYLEARQDELADWTLKSHKYRLRAFVEWCEESGVDDLTELDGRDLYEFRVWRREGNFGVEDGETPEEIAPVTLKSQLTTLRAFLRFAANIHAVPEDFYERVPLPKLSGTDDVSDSTLEPDRATDILEYLHRYHYASRRHVEFALLWETGARMGAIRGLDLRDLDLDGRTPVVRYKHRPDQGTPIKNGEKGERFNSVSDRVGTMLQAYIDGPRVDKTDEFGRKPLLTTSHGRVSASTIRQDVYVVTRPCWLNQGCPHNRDIETCEAVELNHVSTCPSSRSPHDVRKGVVTLYRREEVPRRVVSDRLDASDLVLDKHYDRRGERERAEQRRNHLPW

SEQ ID NO: 135 - protein, 349 aa, Unknown
  Natrinema sp. J7-2
MVIGMSDDLEPIGPEQAVEMYIEGRRDELSDQTLPSHVYRLEAFTQWCAEEGIENLNEITGRNLYAYRVWRREGNGEGREEVTTITLRGQLATLRAFLRFCADIDAVPEDLFSKVPLPTVSASEGVSDTTLEPDRAVEILDYLQRYEYASRKHITLLLLWHTGARAGGVRGLDLRDCELEGESPGLQFVHRPETDTPLKKGEKGERWNSISGHVAGVLQDYVDGPRDNVTDDHGRSPLLTTRSGRPCISTIRDTMYGLTRPCWRGAECPHDRDPEECEATYYAKASTCPSSRSPHDVRSGRVTAYRREDVPRRVVGDRLDASDDILDRHYDRRNAREKAEQRRDYLPDL

SEQ ID NO: 136 - protein, 362 aa, Unknown
  Halostagnicola larsenii XH-48
MSELEPLSPLEALELWLERLQSTRSEATIESYRYRMQSFVEWCDEEEIDNLNDLTSRDVFRYDSERRSEGLSPATLKTQLGTLKLFLEFCDRLEAVPEGLYEKVEVPTVELAERVNDELVRAERAEQILEDLELYDRASRRHAIFAIAWHCGCRLGGLRALDLEDCFFEPSDLDRLRHQDDIDHEALEEVDLPFLYFRHRPETDTPLKNKKQGERPVALSDDVASLIKSYIQVKRAKRSDGDRRPLFTTEKGDNARVSKSSIRRDIYILTQPCRYGTCPHNRDEENCEALKHGHEARCPSSRSPHPIRTGAITHMRDEGWPPEVVAERVNATPEVIRAHYDHPDPIRRMQSRRSFLNKEADT

SEQ ID NO: 137 - protein, 337 aa, Unknown
  Halorhabdus utahensis DSM 12940
MSEDLQPLPPKEGVDRFLEHRAPSIRESSMQNARHRLSVFLEWCDENDVDDLNDLTGRDLSAFVAWRQGDVAAITLQKQLSSVRMALRWWADIEGVEEGLAEKLHSPDLPDGAESKDVFLEADRAKRALRYYDRHHYASRDHALLALIWRTGMRRGAVRGLDVDDLDSDDQAIRVEHRPDTGTPLKNGDGGNRWVYLGPRWFTILEDFVANPDRKNVRDEHGRRPLFTTQQETRPTGHSIYKWVIRALHPCKYAECPHDRKPSECEALGSSSVPSKCPSARSPHSIRRGAITNHLNEETAPETVSERMDVSLDVLYQHYDARTEREKMAVRRHNLPE

SEQ ID NO: 138 - protein, 362 aa, Unknown
  Natronomonas pharaonis DSM 2160
MSRNRSREAPSEWSPRNAAERYIKHRASDTTESSRSGWWYRLKLFVEWCEEVGLETVSDIQPLDIDEYHDIRAEAVAPVTLEGEMATLQEYLRYLEGLDAVADDLSEAVHVPNLDASQRSNDVKLSTPEAMAMLQYFRETPAVRASRKHVFLELVWFTGARQSGLRALDLRDVHLDDAFVWFKHRPSEGTGLKNNLDGERPVSLPSGVVDVLREYIHENRNSETDVHGRAPLFTTLQGRPSGDSVRKWCYLATLPCLHSDCPHGKDRESCDWTGYKYASKCPSTRSPHRIRTGSITYQLNIGFPTEVVANRVNASPKTIRDHYDKADRQERRRRQRRRMESDRRGYVQQMDFDYENDIGSDD

SEQ ID NO: 139 - protein, 349 aa, Unknown
  Natronomonas pharaonis DSM 2160
MSDDLEPIAPAEAVEMYIEARQDDCTENTIEGQYYRLQAFLAWCDEEDITNLNELDGRDLYAYRVWRREGGYSDTELAGATLRGDLATLRAFLRFCGEVEAVPPEFFDRVPLPSVSGGADVSASTLDPDRAQAILEYLQQFEYASKRHVIVLLLWHAGCRVGALRALDVDDLDLAGDRPNATGPGIKFVHRPDEGTPLKNKRKSERWNTISEGVANVIEDYIASRRTEAEDDYGRRPLISTRYGRMSRSAIRQELYRVTRPCWYNDGCPHDRDPDECEATDDGSMSKCPSSRSPHDVRSGRLTFYRLREVDEKVVSDRMDASEEILDKHYDRRSERQKAEQRRSHLPDV

SEQ ID NO: 140 - protein, 345 aa, Unknown
  Haloterrigena thermotolerans DSM 11522
MGDDLEPIAPEQALEMYVEGRRDELSDQTLPSHVYRLEAFTQWCEEEGIENLNTLTGRDLYAYRVWRREGNGDGRDEVATVTLRGQLATLRAFLQFCADIDAVPEELYSKVPLPSVSASEGVSDTTLDPERAVEILDYLQRYEYASRRHVTVLLLWHTGARAGGIRALDLRDCELEGESPGVQFVHRPETDTRLKKGEKGERWNSISGHVAGVLLDYVEGPRKDVTDDHGRSPLLTTRSGRPSVSTIRNTMYGVTRPCWRGAECPHDRDPEDCDATYYAKASTCPSSRSPHDVRSGRVTAYRREDVPRRVVGDRLDASDDILDRHYDRRNAREKAEQRRDYLPDL

SEQ ID NO: 141 - protein, 330 aa, Haloarcula vallismortis
MYLKARQDELTESTIQSQEYRLEAFEQFCSEEGIENLNDLSGRDLYAYRVWRREGNGKEREGIEPITLRGQLATVRSFLRFAAEVDAVPENLRTKVPLPTINGAGEVSASTLDPERADVILDYLQMYKYASRTHVIVLLLWHTGARMGAIRGLDIDDCELEGSDPGIEFVHRPQSDTPLKNGEKGQRWNAISEHVANVVQDYINGPRESVFDEHGRRPLITTQQGRASTSTYRMAMYRVTRPCWRGAECPHDRDPEECEATSNKKASTCPSARSPHDVRSGRVTAYRREDVPRRVVSDRLDASDQILDKHYDRRGEREKSEQRRDYLPEV

SEQ ID NO: 142 - protein, 335 aa, Sulfolobus virus 1
MTKDKTRYKYGDYILRERKGRYYVYKLEYENGEVKERYVGPLADVVESYLKMKLGVVGDTPLQADPPGFEPGTSGSGGGKEGTERRKIALVANLRQYATDGNIKAFYDYLMNERGISEKTAKDYINAISKPYKETRDAQKAYRLFARFLASRNIIHDEFADKILKAVKVKKANADIYIPTLEEIKRTLQLAKDYSENVYFIYRIALESGVRLSEILKVLKEPERDICGNDVCYYPLSWTRGYKGVFYVFHITPLKRVEVTKWAIADFERRHKDAIAIKYFRKFVASKMAELSVPLDIIDFIQGRKPTRVLTQHYVSLFGIAKEQYKKYAEWLKGV

SEQ ID NO: 143 - protein, 328 aa, Unknown
  Sulfolobus spindle-shaped virus 2
MPNFYVGSKFYVKEIKGKYYVYSIENGDDGKQRHTYIGSLEQIVNEYYDMKCGRRDLNPGSPAWEAGIRGTPPKTPDANDDELKGVRIIDSNLTSSNNSEISASDLLKFEFTLRQKKITDKTIKEYINCVKQGRKESNNCIKAWRNFYKLVLNRDPPESLKIKRTKPDLRVPTLEEVRKTLSTVKEYPNLYLFYRLLLESGSRESEALKVLNDYNPQNEIREEGFSIYILNWTRGQKKSFYIFHVTELKQIKISKAYVDKYVRRLNLVPPKYIRKFFATKALELGIPSEVVDFLEGRTPGDILTKHYLDLLTLAKKYYPLYAEWLYTF

SEQ ID NO: 144 - protein, 355 aa, Unknown
  Sulfolobus virus Ragged Hills
MEFLSSSFSLTGDKIIIILFKCLRDKYKWAEGMGNKVFTFGDIRIREVKGKYYVYLIEKDNEGNRRDHYVGSLDQIVKDYISIKVRGTGFEPAQAFASGASVRPMGDTPIPPDLKNKGVITKDMEITRDKLNEFFEWCVKKRKNSIDTCKDYILYLKRPLNKNKKWSVFAYRLYYEFLGKEDKAKELKVEKKMSIPVYRIPSLEEIKKVLNHEDERIRILYRLLLESGIRLKEALFILNNYDPALDQMEDGFYVYTVNLIRKSKKSFYAFHITPLQKTYITESIIDHTDLPVKPKFIRKFVATKMLELGIPSEVVDFFQGRTPSSILSKHYLDLLTLAKKEYKKYAEWLTKYVLL

SEQ ID NO: 145 - protein, 340 aa, Unknown
  Sulfolobus virus Kamchatka 1
MPSFYVGSNFYIKEIKGKYYVYSIEKGEDNKQRHHYIAPLDKVIEFYISNGGLRGYPPNGGVGVPPTMGACRAPDPGSNPGRGAFLYVDSNNELKGVRIIDSNLTSSNNSEISASDLLKFELTLRQKNISEETIKKYISCVKQGRKESNNCIKAWRNFYRLVLNRDPPSELKPKKTKPDLKVPTLEEVRETLDKVKQYPSLYLLYRLLLESGSRLREALKLLNNYNPQNEIRGDGFSIYVLNWTRGQKKSFYLFHITELKAEKVTEGQITSAVRRLNLVPPKYIRKFVATKLFELGVSSEVVDFLEGRTPGNILTKHYLDLLTLAKKEYKKYAEWLKQII

SEQ ID NO: 146 - protein, 347 aa, Unknown
  Acidianus spindle-shaped virus 1
MATIILGDKMAKDKTRYKYGDIILRERKGRYYIYKLETINGETKETYVGPLIDVVESYLKMKEIGVLGVSPNVAGPPGFEPGTYGLKARRELDELRDRAEELKEVAILRKYVTEGNLEEFYSWATMKKGIDERTAKLYVRQIQKPFEKKRNRIFAYRAFARFLIEKGIGVSDILEKLKTISSKPDLRVPTLDEVRKTLQLAKEYSENVYFVYRLALESGSRLSEILKVLKEPEKDVCDNDICYYPLAWTRGQKSVFYVFHLTPLRKIDITQWAISDFERRNDEAIPIKYIRKFVATELAGLGINFDIIDFIQGRKPSRVLTQHYVSMFAIAKENYKKYAEWIRQTLT

SEQ ID NO: 147 - protein, 354 aa, Unknown
  Sulfolobus spindle-shaped virus 6
MIVISLFKHQRDNYKWAEGMGNKVFTFGDIRIREVKGKYYVYLIEKDNEGNRRDNYVGKLDEVVRFYIKNAKTGVVGAFPPQGSGPWDQGSNPCPATFLSPLSNNELNVVITNEASFTGDKKTEKLPSEMELFAFYNDCVKKVSRETCKEYVNYLRKPLDVNNKASILAWKKYYKWKGDLEAWKKIKTKKSGVDLRVPSEAEIKEWLTKVKGTKVELLFKLLLESGIRLTEAVKLVNEYDPKNETIESSYYIYTMNWSRGSKRVFYVFHVTPLQKLQITYNYAKKLFHELKIDPKYVRKFVATKCLELNIPAEVVDFLEGRTPTQILTRHYLDLLTLTKKYYPLYAEWLRQTLT

SEQ ID NO: 148 - protein, 336 aa, Unknown
  Sulfolobus spindle-shaped virus 7
MPNFYVGSKFYVKEIKGKYYVYSIENGDDGKQRHTYIGSLEQIITSYLELGVWGVPPQCGRRDLNPGSPAWEAGIRGAPPKTPTDNNVELKGVRIIDSNLTSSNNSEISVSDLIKFEFALRQKKITDKTIKEYLSCIKRNKKDSNNCIKAWRNFYRLVLNRDPPESLKIKRTKPDLRVPTLEEVRKTLSTVKEYPNLYLFYRLLLESGSRESEALKVLSEYNSQNEMQEVGFSIYILNWTRGQKKSFYLFHVTELKQIKISKAYVDKYVKKLNLTPPKYIRKFTATKMLELGIPSEVVDFIQGRTPSEVLTKHYLDLLTLAKKEYKKYAEWLRQNI

SEQ ID NO: 149 - protein, 323 aa, Unknown
  Sulfolobales Mexican fusellovirus 1
MADKPRTVTLGEFRLRYLKNKVYVYKVKNGYEEEYIAPLERLVEHFLSTADAKGQDRKDGKGQIDVLQSAPENVGETKVNRNEVTVSSVIELQRFFNWCVKFASEQTCNTYVKYLQRPPNSTHPSIRAWRAYYKWKGKEDKLKELKLPRSGSDLRLVTEDEVKRALKNSSGDEVAHYILSLLVESGLRLSEVVKVLNEYEPSQDTAYNTFNVYNVNWRRGRKNTLYMFHISPLRQMTLDYENTRVKLARYIDAKFMRKFVATKMFELEIPAEVIDFIQGRAPTTVATKHYIYLFTIARKYYEEKWVPYVRALLNLNSQGESKT

SEQ ID NO: 150 - protein, 399 aa, Unknown
  Aeropyrum pernix ovoid virus 1
MWGEPLLYGAGDSTVTLVPKPLYVYVHTVKSKGRIYQYLVVEEYLGQGRRRTILRMRLEEAVRKLLNNEKKDSAETAGWCGGWDLNPRRPTPTGLKPAPSKPFSSMVIEKRDSGDGESEPSFKQDGGLIVSETLASRFLEWLDLPEDSRQLRDYRNNLRLLIGKPLDCATLHEFASQSKRKYETASRLLSFVASKRGLGLRQLAAELRECLGKKPRSGSDTYVPPDSSILEAARRLEGTRVYHVFLLLVGSGARLSTVHWLLRQGLDSSRLVCLEDRGFCRYHVDYVKGEKLQWALYSPREFWERVLEEPRLTLSYNRVQEQIAGAGVKAKHIRNWVYNKMLSLGMPEGVVEFIVGHKASSIGRRHYMNMIVQADMWYTTYLPVIPKSLKLSCTTCYEG

SEQ ID NO: 151 - protein, 291 aa, Sulfolobussolfataricus
MKLDLGSPPESGDLYNAFINALIIAGAGNGTIKLYSTAVRDFLDFINKDPRKVTSEDLNRWISSLLNREGKVKGDEVEKKRAKSVTIRYYIIAVRRFLKWINVSVRPPIPKVRRKEVKALDEIQIQKVLNACKRTKDKLIIRLLLDTGLRANELLSVLVKDIDLENNMIRVRNTKNGEERIVFFTDETKLLLRKYIKGKKAEDKLFDLKYDTLYRKLKRLGKKVGIDLRPHILRHTFATLSLKRGINVITLQKLLGHKDIKTTQIYTHLVLDDLRNEYLKAMSSSSSKTPP

SEQ ID NO: 152 - protein, 286 aa, Metallosphaera sedula
MKLQLGEPPTDADPFIYFMESLKFSGAGQGTIKLYSTAIQDFLQFVKKDPRSVTTQDVIDWIGSLNSRKGRSRVVDKRGRSATIRSYVIAVRRFLKWLGVNVKPPVPRIRSPERMALREEDIVALLSACRRLRDKVIVSLLVDTGLRSSELLSLRRSDVDLERMLIRVRETKNGEERIVFFTSRTATLLRQYLRKTQDKESDDAPLFNLSYQALYKLIKRLGRKTGLTWLRPHVLRHTFATNAIRRGVPLPAVQRLMGHKDIKTTQIYTHLVTEDLENAYRRAFET

SEQ ID NO: 153 - protein, 283 aa, Thermoplasma acidophilum
MPAETNEYLSRFVEYMTGERKSRYTIKEYRFLVDQFLSFMNKKPDEITPMDIERYKNFLAVKKRYSKTSQYLAIKAVKLFYKALDLRVPINLTPPKRPSHMPVYLSEDEAKRLIEAASSDTRMYAIVSVLAYTGVRVGELCNLKISDVDLQESIINVRSGKGDKDRIVIMAEECVKALGSYLDLRLSMDTDNDYLFVSNRRVRFDTSTIERMIRDLGKKAGIQKKVTPHVLRHTFATSVLRNGGDIRFIQQILGHASVATTQIYTHLNDSALREMYTQHRPRY

SEQ ID NO: 154 - protein, 286 aa, Pyrococcus furiosus
MREKTLRSEVLEEFATYLELEGKSKNTIRMYTYFLSKFLEEGYSPTARDALRFLAKLRAKGYSIRSINLVVQALKAYFKFEGLNEEAERLRNPKIPKTLPKSLTEEEVKKLIEVIPKDKIRDRLIVLLLYGTGLRVSELCNLKIEDINFEKGFLTVRGGKGGKDRTIPIPQPLLTEIKNYLRRRTDDSPYLFVESRRKNKEKLSPKTVWRILKEYGRKAGIKVTPHQLRHSFATHMLERGIDIRIIQELLGHASLSTTQIYTRVTAKHLKEAVERANLLENLIGGE

SEQ ID NO: 155 - protein, 282 aa, Unknown
  Thermococcus kodakarensis
MSEPNEVIEEFETYLDLEGKSPHTIRMYTYYVRRYLEWGGDLNAHSALRFLAHLRKNGYSNRSLNLVVQALRAYFRFEGLDDEAERLKPPKVPRSLPKALTREEVKRLLSVIPPTRKRDRLIVLLLYGAGLRVSELCNLKKDDVDLDRGLIVVRGGKGAKDRVVPIPKYLADEIRAYLESRSDESEYLLVEDRRRRKDKLSTRNVWYLLKRYGQKAGVEVTPHKLRHSFATHLLEEGVDIRAIQELLGHSNLSTTQIYTKVTVEHLRKAQEKAKLIEKLMGE

SEQ ID NO: 156 - protein, 278 aa, Unknown
  Methanocella arvoryzae
MCMGIGMDYVAVFIDEKRLSSSPGTIRQYGMILNRFYKYTGKQPEMVVRPEIVRYLNYLMFEKHLSKTTVANVLSVLKSFYSFMLDNGYVSSNPTRGINNIKLDKKAPVYLTVSEMNDLLDTAIDTRDRIIVRLLYATGVRVSELVNIRKKDIDFDRCTIKVFGKGAKERIVLVPETVVKEMYDYAASLSNDDRLFNLTPRTVQRDIKQLARRAKINKNVTPHKLRHSFATHMLQNGGNVVAIQKLLGHSSLNTTQIYTHYNVDELKEMYGRTHPLGK

SEQ ID NO: 157 - protein, 284 aa, Unknown
  Aciduliprofundum boonei
MSDKFMDYVDYELEKFKEYLRGEKRSENTIKEYAHFISDMLRYFHKRAEDITPGDLNKYKMYLSTKRKYSKNSLYLATKAIRSYFKYKNLDTAKNLSSPKRPRQMPKYLSEDEVKRLIEASSENPRDYAIISLLAYSGLRVSELCNLKIEDVDFNERIVYVHSGKGDKDRIVVVSPRVIEALQNYLYTREDDMEYLFASQKSNKISRVQVFRIVKKYAEKAGIKKEVTPHVLRHTLATTLLRRGVDIRFIQQFLGHSSVATTQIYTHVDDALLKSVYDKVLQEY

SEQ ID NO: 158 - protein, 278 aa, Unknown
  Thermococcus nautili
MDEVIEEFETYLDLEGKSPNTIRMYSYYVRRYLEWGGALNARSALRFLARLRREGYSNRSLNLVVQALRAYFRFEGHDEEAEKLKPPKVPRSLPKALTREEVKRLLSVIPPTRKRDRLIVLLLYGAGLRVSELVNLKKSEVDLERGIIVVRGGKGAKDRVVPIPEFLVEEIRSYLETRSDSSEYLLVEERRKNKDRLSTKTVWYLLKKYGKRAGVEVTPHRLRHSFATHMLERGVDIRAIQELLGHSNLSTTQIYTKVTVEHLRKAQEKARLMEGLVE

SEQ ID NO: 159 - protein, 302 aa, Vibrio cholerae
MSEALSPDQGLVEQFLDTMWFERGLAENTVASYRNDLSKLLEWMAQNQYRLDFISFAGLQEYQSWLSEQNYKPTSKARMLSAIRRLFQYLHREKVRADDPSALLVSPKLPTRLPKDLSEAQVEALLSAPDPQSPLELRDKAMLELLYATGLRVTELVSLTMENMSLRQGVVRVMGKGGKERLVPMGENAIEWIETFLQQGRSLLLGEQTSDIVFPSSRGQQMTRQTFWHRIKHYAVIAGIDVEKLSPHVLRHAFATHLLNYGADLRVVQMLLGHSDLSTTQIYTHVATERLKQLHNEHHPRA

SEQ ID NO: 160 - protein, 298 aa, Escherichia coli
MKQDLARIEQFLDALWLEKNLAENTLNAYRRDLSMMVEWLHHRGLTLATAQSDDLQALLAERLEGGYKATSSARLLSAVRRLFQYLYREKFREDDPSAHLASPKLPQRLPKDLSEAQVERLLQAPLIDQPLELRDKAMLEVLYATGLRVSELVGLTMSDISLRQGVVRVIGKGNKERLVPLGEEAVYWLETYLEHGRPWLLNGVSIDVLFPSQRAQQMTRQTFWHRIKHYAVLAGIDSEKLSPHVLRHAFATHLLNHGADLRVVQMLLGHSDLSTTQIYTHVATERLRQLHQQHHPRA

SEQ ID NO: 161 - protein, 298 aa, Escherichia coli
MTDLHTDVERYLRYLSVERQLSPITLLNYQRQLEAIINFASENGLQSWQQCDVTMVRNFAVRSRRKGLGAASLALRLSALRSFFDWLVSQNELKANPAKGVSAPKAPRHLPKNIDVDDMNRLLDIDINDPLAVRDRAMLEVMYGAGLRLSELVGLDIKHLDLESGEVWVMGKGSKERRLPIGRNAVAWIEHWLDLRDLFGSEDDALFLSKLGKRISARNVQKRFAEWGIKQGLNNHVHPHKLRHSFATHMLESSGDLRGVQELLGHANLSTTQIYTHLDFQHLASVYDAAHPRAKRGK

SEQ ID NO: 162 - protein, 306 aa, Unknown
  Caldithrix abyssi
MDKHIRDFLRYLFLERRYARNTIRSYGTDLLQFEEFLEQHFTATNIPWSLVDKRVIRFFLIRLQEQKISKRSIARKLATLKSFFRYLLKNGIIESNPVATVKMPKLEKKLPEHLGPAEIEALLRLPKLNTFEGLRDLAILELFYGTGIRLSELINLKVSQVDFQENLIRVIGKGNKERIVPFGGSAKLILEKYLSIRPQFAENSVDNLFVLKSGKKMYPMAVQRIVKKYLTQASNLKQKSPHVLRHTYATHLLNQGADIRVVKDLLGHENLATTQIYTHLSIEHLKKVYNQAHPRATNKSSKNRRR

SEQ ID NO: 163 - protein, 306 aa, Unknown
  Shewanella baltica
MSTQTAEVSALNTQWLQTFERYLSTERQLSAHTVRNYLYELNRGSDLLPDGVNLLNVSREHWQQVLAKLHRKGLSPRSLSLCLSAVKQWGEFLLREGVIELNPAKGLSAPKQAKPLPKNIDVDAISHLLDIEGTDPLSLRDKAMMELFYSSGLRLAELAALNLSSVQYDLKEVRVLGKGNKERIVPVGRLAIAALLNWLNCRKQIPCEDNALFVTEKGKRLSHRSIQARMAKWGQEQALSVRVHPHKLRHSFATHMLEASADLRAVQELLGHANLATTQIYTSLDFQHLAKVYDNAHPRAKKTQDK

SEQ ID NO: 164 - protein, 308 aa, Unknown
  Desulfococcus oleovorans
MSKDHGAYPAKPLADAFVESLASEKGYSPNTCRAYSADLKEFLAFLSPPDDTEHPVCLDDISVIAIRGYLAFLHKKKMDKSTVSRKLSVLRSFFRYLEKRGIMTGNPARAVLSPKIGRKIPAFLSVDDMFRLLDASTGDTLLDLRNRAIFETIYSTGIRVSEAAGLDAAHVETDERVFRVYGKGAKERVVPVGKKALASIAAYRTRLFEETGIGVEEGPLFLNKNRGRLTTRSMDRILKQTALRCGLTVSLSPHALRHSFATHMLDAGADLRTVQEILGHKSLSTTQKYTHVSMDKLMEVYDHAHPRK

SEQ ID NO: 165 - protein, 296 aa, Unknown
  Salinicoccus luteus
MNFKRYIEEYLLFLSVEKGLSQSSISSYRQDLMQYEAFLSDHSALDPSQIDTELLIRFLKELRHAGKSAKTISRMQSTLKNFHQFLVNDGITTHNPALRLHSIKEAKKLPVYLTVEEMEKLLSTPDQSVAGVRDKSMMELLYASGLRVSELIDIRTSDLNTDMGYIRIMGKGSKERIVPITDFVGELLEQYMSNERMALLKDDVVEELFITNRGRGFTRQGLWKTIKKYELASGIGKNITPHTFRHSFATHLVENGADLRAVQEMLGHSDISTTQIYTQISAVKIREMYKKFHPRK

SEQ ID NO: 166 - protein, 307 aa, Unknown
  Dehalococcoides mccartyi
MQENFNKYLEYLTVEKNVSVYTLRNYRTDLIGFINYLIEKKVSSFDRVDRYILRDYMSSLIEKGIVKGSIARKLSAVRSFYRYLMREGLIQKNPTLNASSPRLDKRLPEFLTTAEVSKLLRIPDSSTPQGLRDKAFMELLYASGLRVSELVKLDIENLDLHSHQIRVWGKGSKERIVLMGLPAIQSIQTYLNLGRPLLKSKRNTPALFLNPNGGRLSARSFQERLDKLAHQAGIEKHVHPHMLRHTFATHLLDGGADLRVVQELLGHSNLSTTQIYTHVTKSQARKVYMSSHPLAKPQNDISGSEDE

SEQ ID NO: 167 - protein, 296 aa, Bacillus pumilus
MNDQLSDFIHFMTVERGLSENTIVSYKRDLQNYLSFLMTHEQLTDIKDVTRLHIIHYLKQLKEEGKSSKTSVRHLSSIRSFHQFLLREKVTTDDPSWNIETQKTERKLPKVLSLEEVEKLLDTPNQHTPFDYRDKAMLELLYATGIRVSEMLDLTLADVHLTMGFIRCFGKGRKERIVPIGEACASAIEEYLEKGRSKLLKKQPADALFLNHHGKKMSRQGFWKNLKKRALEAGIQKELTPHTLRHSFATHLLENGADLRAVQEMLGHADISTTQIYTHVTKTRLKDVYHKFHPRA

SEQ ID NO: 168 - protein, 300 aa, Klebsiella aerogenes
MSHSPLFACVDRFLRYLGVERQLSPITLTNYQRQLEALIALADDAGLKSWQQCDAAQVRSFAVRSRRAGLGPASLALRLSALRSFFDWMVSQGELAANPAKGIAAPKIPRHLPKNIDVDDVNRLLDIDLNDPLAVRDRAMLEVMYGAGLRLSELVNLDIQHLDLESGEVWVMGKGSKERRLPIGRNAVAWIEHWLDLRGLFGGDDDALFLSKLGKRISARNVQKRFAEWGIKQGLNSHVHPHKLRHSFATHMLESSGDLRGVQELLGHANLSTTQIYTHLDFQHLASVYDAAHPRAKRGK

SEQ ID NO: 169 - protein, 299 aa, Staphylococcus carnosus
METNYDVVIEEYLKFIQIEKGLSANTIGAYRRDLNKYKEYLVLKKINNIDFIDREIIQQCLGYLHDDGHSAKSIARFISTVRSFHQFALRERYAAKDPTVLIETPKYERRLPDVLDVEDVLALLETPDLSKNNGYRDRTILELLYATGMRVTELIHVRVEDVNLIMGFVRVFGKGSKERIIPLGETVIDYLKKYIETVRPQLLKQAVTDVLFLNLHGKPLSRQGIWKLIKQYGVKANIKKKLTPHSLRHSFATHLLENGADLRAVQEMLGHSDISTTQLYTHVSKSQIRKMYNEFHPRA

SEQ ID NO: 170 - protein, 302 aa, Unknown
  Dickeya solani
MNPDSPLSAPAEAFLRYLRVERQLSPLTQSSYAHQLQVIIDMLSASGITDWQALDAAGVRAVVARSKRDGLNAASLAQRLSALRSFLDWLVGRGELKANPARGVPAPKAGRHLPKNMDVDEMSRLLDIDLSDPLAVRDRAMLEVMYGAGLRLAELVGLDCGHVDLDSGEVWVMGKGSKERKLPIGATAVTWLRHWLAIRDIYAPEDDAIFISSLGKRISMRNVQKRFAEWGVKQGVNSHVHPHKLRHSFATHMLESSGDLRAVQELLGHANLSTTQIYTHLDFQHLASVYDAAHPRARRGKP

SEQ ID NO: 171 - protein, 304 aa, Unknown
  Fervidicola ferrireducens
MEYEVVDSFLNYIKAAKNQSENTLKAYANDLGQFIEYLEQNKMSETKSLKNITHLDIRGFLAYLKEKGVAKKSITRKLSALRSFFKYLTTEGIISEDPTKMVQGMKLPKKLPLFLYPAEIEALLSAPKNDVLGIRDRAIMELLYATGVRVGELVSIKLKDVNMGANFIIVYGKGSRERMVFFGSKAAESLEEYLKKSRPYLVKNLSCEYLFLNKNGTRLTDRSVRRIIDKYVKELSLNKNISPHTLRHTFATHMLNNGADLKTVQELLGHVSLSTTQLYTHVTKERLKEIYDKVFPRAKKKEES

SEQ ID NO: 172 - protein, 308 aa, Unknown
  Pragia fontium
MSERTEPLTCPSLQQPVDNFLRYLRVERQLSPYTLKSYQRQLAALIDLLVNIGLTDWTKLDAAGVRMLVTRSKRSGLESASLALRLSALRSFLDWLVGQGIIGANPAKGISTPRKGRHLPKNMDVDEVNHLLDIDLNDPLAVRDRTMLELMYGAGLRLSELIGLDCRQVNLDAGEIRVVGKGSKERKLPIGRMAVTWLNRWLPMREFYAPDDDALFVSKHGNRISARNVEKRFAEWGVKQGISSHVHPHKLRHSFATHMLESSGDLRAVQELLGHANLTTTQIYTHLDFQHLTKVYDAAHPRAKRGKP

SEQ ID NO: 173 - protein, 303 aa, Syntrophomonas wolfei
MLLFQYIEAFLNHMRVEKSASNFTLSSYKTDLSQFFAFLSQKKGINPEEVGVELINHNSVRKYLAQMQEKGLSRATMARKLAALRSFIKFLCRENILADNPITAVSTPKQERKLPRFLYTREMELLMNAPDLSMAAGKRDRAILETLYASGLRVSELTNLDKPDIDFGEDYIKVLGKGGKERIVPLGSKAREALLLYLQQGRVYLEAKGQASPALFLNKNGQRLSTRSIRNIINKYVETIAINQKVSPHTLRHSFATHLLNNGADLRSVQELLGHVKLSTTQIYTHLSREKIKDIHQQTHPRR

SEQ ID NO: 174 - protein, 317 aa, Sporomusa sphaeroides
MMIMCDNKQTNQIDKFIDQFMFYLRVEKNSSRHTLLNYQRDIYQFVEFVSNQGGGERPFSYVTPLLLRSYLAHLKSQEYAKATIMRRIAALRSFFRFLCRENILSENPCDAVRTPKLEKKLPVFLDANEVSELMALPDDSPLGFRDKAVLELLYATGVRVNELAGITLPDIDVEGRTIIVSGKGAKERIVLMGKTAAAFLEKYLQRARPVLCTKTGEYGRQTKKQHSYLFVNNRGGPLTDRSIRRIVEKYVEEMALKKNVSPHTLRHTFATHLLDNGADLRTVQELLGHVNLSTTQLYTHITTERLKANYKKSHPRA

SEQ ID NO: 175 - protein, 362 aa, Helicobacter pylori
MKHPLEELKDPTENLLLWIGRFLRYKCTSLSNSQVKDQNKVFECLNELNQACSSSQLEKVCKKARNAGLLGINTYALPLLKFHEYFSKARLITERLAFNSLKNIDEVMLAEFLSVYTGGLSLATKKNYRIALLGLFSYIDKQNQDENEKSYIYNITLKNISGVNQSAGNKLPTHLNNEELEKFLESIDKIEMSAKVRARNRLLIKIIVFTGMRSNEALQLKIKDFTLENGCYTILIKGKGDKYRAVMLKAFHIESLLKEWLIERELYPVKNDLLFCNQKGSALTQAYLYKQVERIINFAGLRREKNGAHMLRHSFATLLYQKRHDLILVQEALGHASLNTSRIYTHFDKQRLEEAASIWEEN

SEQ ID NO: 176 - protein, 371 aa, Streptomyces coelicolor
MGETGRQLAVVTADADVVEAELVDDETAGASVVVHTDRDRHLSPETVAAIAASVADSTRRAYGTDRAAFAAWCAEEDRTAVPASAETMAEWVRHLTVTPRPRTQRPAGPSTIERAMSAVTTWHEEQGRPKPNMRGARAVLNAYKDRLAVEKAEAAQARQATAALPPQIRAMLAGVDRTTLAGKRNAALVLLGFATAARVSELVALDVDTVTEAEHGYDVTLYRKKVRKHTPNPILYGTDPATCPVRALRAYLAALAAAGRTDGPLFVRVDRWDRLAPPMTRRGRVIGDPAGRMTAEAAAEVIERLAVAAGLSGDWSGHSLRRGFATAARAAGHDPLEIARAGGWVDGSRVLARYMDDVDRVKNSPLVGIGL

SEQ ID NO: 177 - DNA, 142 bp, Artificial Sequence
  Synthetic Polynucleotide
cacccagggcatccccgacttctttaagcagtccttccctgaggtaagttcggccggcttgtcgacgacggcggactcagtggtgtacggtacaaacccgtgctcgcttcggcagcacatatactatgttgaatgaggcttc

SEQ ID NO: 178 - DNA, 142 bp, Artificial Sequence
  Synthetic Polynucleotide
gaagcctcattcaacatagtatatgtgctgccgaagcgagcacgggtttgtaccgtacaccactgagtccgccgtcgtcgacaagccggccgaacttacctcagggaaggactgcttaaagaagtcggggatgccctgggtg

SEQ ID NO: 179 - DNA, 143 bp, Artificial Sequence
  Synthetic Polynucleotide
  misc_feature (107)..(108): n is a, c, g, or t
cacccagggcatccccgacttctttaagcagtccttccctgaggtaagttcggccggcttgtcgacgacggcggactcagtggtgtacggtacaaacccgtgctcgnnttcggcagcacatatactatgttgaatgaggcttc

(19) United States
(12) Patent Application Publication
(10) Pub. No.: US 2022/0267802 A1
(43) Pub. Date: Aug. 25, 2022

(54) METHODS AND COMPOSITIONS FOR GENE DELIVERY

(71) Applicant: President And Fellows Of Harvard College, Cambridge, MA (US)

(72) Inventors: Benjamin Weinberg, Cambridge, MA (US); Denitsa M. Milanova, Cambridge, MA (US); Issac Han, Cambridge, MA (US); George M. Church, Cambridge, MA (US)

(73) Assignee: President And Fellows Of Harvard College, Cambridge, MA (US)

(21) Appl. No.: 17/627,229

(22) PCT Filed: Jul. 14, 2020
(86) PCT No.: PCT/US20/41950

§ 371 Date: Jan. 14, 2022

Related U.S. Application Data
(60) Provisional application No. 62/874,241, filed on Jul. 15, 2019.

(51) Int. Cl. C12N 15/864; C12N 15/90; C12Q 1/686

(52) U.S. Cl. CPC C12N 15/8645; C12N 15/90; C12Q 1/686; C12N 2750/14143

(57) ABSTRACT

Provided herein, in some embodiments, are methods and compositions for gene delivery. Provided herein is a technology for co-delivering to a cell (e.g., in vivo or ex vivo) enzymes capable of rearranging nucleic acid, such as site-specific recombinases, to directly assemble (e.g., covalently join) nucleic acid segments of, for example, a gene of interest.

### RELATED APPLICATION

This application claims the benefit under 35 U.S.C. § 119(e) of U.S. provisional application No. 62/874,241 filed on Jul. 15, 2019, which is incorporated by reference herein in its entirety.

### FEDERALLY SPONSORED RESEARCH

This invention was made with government support under DE-FG02-02ER63445 awarded by the Department of Energy. The government has certain rights in the invention.

### BACKGROUND

The delivery of nucleic acids to cells finds many important applications in human health, biochemical production, and scientific discovery. Some of the most commonly vectors used for gene delivery include lentivirus (LV), retrovirus (RV), herpes simplex virus-1 (HSV-1) and adeno-associated virus (AAV). Nonetheless, the use of vectors for delivering nucleic acids are limited in size capacity. This limitation prevents delivery of large genes or other large nucleic acid sequences that are necessary for treatment of diseases and other gene delivery applications.

### SUMMARY

Provided herein is a technology for co-delivering to a cell (e.g., in vivo or ex vivo) enzymes capable of rearranging nucleic acid, such as site-specific recombinases, to directly assemble (e.g., covalently join) nucleic acid segments of, for example, a gene of interest. These enzymes can be programmed to join multiple nucleic acid molecules (e.g., segments) together efficiently in a site-directed and order-specific manner, resulting, for example, in expression of a full length protein encoded by the nucleic acid segments, following a single translation event, without the need for protein engineering. Moreover, site-specific recombinases do not rely heavily on cellular components and machinery, providing a more consistent and tunable assembly strategy across cell types, relative to current strategies that use pre-existing repair machinery encoded in the target cells, which has proven to be inefficient, variable between cell type, and difficult to control.

In some embodiments, the enzyme capable of rearranging nucleic acid is a site-specific recombinase (SSR), which is a small enzyme (e.g., ˜200 to ˜700 amino acids) that catalyzes the transfer and rearrangement of nucleic acids by executing nucleic acid-binding, cutting, transfers and ligation reactions. SSRs carry out these activities on a unique sequence referred to as a recombination site (RS), which is typically between 27 to 250 base-pairs in sequence length. Depending on the placement and orientation of the RS sequences, SSRs can invert, delete, or translocate nucleic acids. SSRs can be classified based on which amino acid residue is primarily responsible for covalent attachment to nucleic acids: tyrosine (tyrosine recombinases) or serine (serine recombinases) residues.

Adeno-associated virus (AAV) vectors have been included in virus-based products federally-approved in the U.S. for in vivo gene therapy of inherited diseases, with many more currently undergoing in clinical trials. Despite much interest around AAV as safe and effective vehicle for gene delivery, AAV cannot package sequences longer than the 4.7 kilobases (kb). More than 4% of the human genes are longer than 4.7 kb, while 11.8% exceed 3 kb (2398 total genes). Thus, in some embodiments, AAV vectors are used to deliver nucleic acid molecules to a cell.

Some aspects of the present disclosure provide a method comprising delivering to a cell (a) a first vector comprising a first segment of a nucleic acid segment and a first recombination site, (b) a second vector comprising a second segment of the nucleic acid and a second recombination site, (c) and a cognate site-specific enzyme or a nucleic acid encoding a cognate site-specific nucleic acid-rearranging enzyme that catalyzes a recombination event to join the first segment to the second segment, thereby forming a transcription product.

In some embodiments, (c) comprises the nucleic acid encoding a cognate site-specific nucleic acid-rearranging enzyme that catalyzes joining of the first segment to the second segment.

In some embodiments, the method further comprises at least one additional vector comprising at least one addition segment of the nucleic acid and at least one addition recombination site.

In some embodiments, the first vector or second vector comprises the nucleic acid encoding the cognate site-specific nucleic acid-rearranging enzyme.

In some embodiments, a third vector comprises nucleic acid encoding the cognate site-specific nucleic acid-rearranging enzyme.

In some embodiments, the first vector comprises a promoter operably linked to the first segment of the nucleic acid. In some embodiments, the third vector comprises a promoter operably linked to the nucleic acid encoding the cognate site-specific nucleic acid-rearranging enzyme.

In some embodiments, the second vector comprise a post-transcriptional regulator element (e.g., woodchuck hepatitis virus post-transcriptional regulator element (WPRE)). In some embodiments, the third vector comprise a post-transcriptional regulator element (e.g., WPRE).

In some embodiments, following the transcription event the transcription product comprises a scar recombination site located between the first segment and the second segment.

In some embodiments, the first vector further comprises a splice donor site and the second vector comprises a branch point site and a splice acceptor site, and following a recombination event, the scar recombination site of the transcription product is flanked by (i) the splice donor site and (ii) the branch point site and the splice acceptor site.

In some embodiments, the first segment, second segment, and/or at least one additional segment are exons of a gene of interest.

In some embodiments, the gene of interest is a therapeutic gene, optionally selected from the group consisting of any of the therapeutic genes listed in Table 1.

In some embodiments, the gene of interest encodes a gene-editing protein, optionally a Cas9 enzyme or a Cas9 enzyme variant (e.g., Cas9 fused to a transcriptional activator, a transcriptional repressor, or a deaminase).

In some embodiments, the first vector, the second vector, and/or the at least one additional vector is selected from the group consisting of lentiviral vectors, retroviral vectors, adenoviral vectors, and adeno-associated viral vectors. In some embodiments, the first vector, the second vector, and/or the at least one additional vector is an adeno-associated viral vector.

In some embodiments, the site-specific enzyme is selected from the group consisting of site-specific recombinases, DDE transposases, DDE LTR-retrotransposases, and target-primed retrotransposases.

In some embodiments, the site-specific enzyme is a site-specific recombinase (SSR) selected from the group consisting of serine recombinases, RKHRY-type recombinases, and HUH-type recombinase.

In some embodiments, the SSR is a serine recombinase selected from the group consisting of small serine recombinases, large serine integrases, and IS607-like serine transposases.

In some embodiments, the serine recombinase is a small serine recombinase selected from the group consisting of resolvases, invertases, and resolvase-invertases. In some embodiments, the small serine recombinase is a resolvase selected from the group consisting of Tn3 resolvase and gamma-delta resolvase. In some embodiments, the small serine recombinase is an invertase selected from the group consisting of Gin invertase and Hin invertase. In some embodiments, the small serine recombinase is a resolvase-invertase selected from the group consisting of BinT resolvase-invertase and beta resolvase-invertase.

In some embodiments, the serine recombinase is a large serine recombinase selected from the group consisting of Bxb1 recombinase, TP901-1 recombinase, PhiC31 recombinase, TG1 recombinase, and PhiRv1 recombinase. In some embodiments, the SSR is Bxb1 recombinase.

In some embodiments, the SSR is a RKHRY-type recombinase selected from the group consisting of tyrosine recombinases, tyrosine integrases, tyrosine invertases, tyrosine shufflons, tyrosine transposases, topoisomerase IB, and telomere resolvases.

In some embodiments, the RKHRY-type recombinase is a tyrosine recombinase selected from the group consisting of Cre recombinase, Flp recombinase, XerC/D recombinase, and XerA recombinase. In some embodiments, the RKHRY-type recombinase is a tyrosine integrase selected from the group consisting of Lambda integrase, P2 integrase, and HK022 integrase. In some embodiments, the RKHRY-type recombinase is a tyrosine invertase selected from the group consisting of FimB invertase, FimE invertase, and HbiF invertase. In some embodiments, the RKHRY-type recombinase is a tyrosine Rci shufflon. In some embodiments, the RKHRY-type recombinase is a tyrosine transposase selected from the group consisting of crypton transposases, DIR transposases, Ngaro transposases, PAT transposases, Tec transposases, Tn916 transposases, and CTnDOT transposases.

In some embodiments, the SSR is a HUH-type recombinase selected from the group consisting of Y1-transposases of IS200/IS605 (e.g., IS608 TnpA and ISDra2), and ISC transposases (e.g., IscA), helitron transposases, IS91 transposases, AAV Rep78 transposases, and TrwC relaxases.

In some embodiments, the site-specific enzyme is a DDE transposase selected from the group consisting of Tc1/mariner transposases, piggyBac transposases, Transib transposases, hAT transposases, Tn5 transposases, P elements, mutator transposases, and CMC transposases.

In some embodiments, the site-specific enzyme is a DDE LTR-retrotransposase selected from the group consisting of Ty3/gypsy and HIV integrase.

In some embodiments, the site-specific enzyme is a target-primed retrotransposase selected from the group consisting of LINE-1 and Group II introns.

In some embodiments, the first vector, second vector, third vector, and/or site-specific nucleic acid-rearranging enzyme are delivered to the cell via electroporation, polymer formulation, or other transfection reagent.

Other aspects of the present disclose provide methods that comprise delivering to a cell at least two viral vectors, each comprising a payload, using a site-specific recombinase. In some embodiments, the viral vectors are adeno-associated viral vectors. In some embodiments, the site-specific recombinase is Bxb1 recombinase.

Further aspects of the present disclose provide a cell comprising the first vector, the second vector, and the cognate site-specific enzyme or the nucleic acid encoding the cognate site-specific nucleic acid-rearranging enzyme of any one of the preceding claims. In some embodiments, the cell is a mammalian cell, optionally a human cell.

Still other aspects of the present disclose provide a composition comprising the first vector, the second vector, and the cognate site-specific enzyme or the nucleic acid encoding the cognate site-specific nucleic acid-rearranging enzyme of any one of the preceding claims and at least one additional reagent (e.g., cell culture media or buffer).

Yet other aspects of the present disclose provide a kit comprising the first vector, the second vector, and the cognate site-specific enzyme or the nucleic acid encoding the cognate site-specific nucleic acid-rearranging enzyme of any one of the preceding claims and at least one additional reagent (e.g., cell culture media or buffer), wherein the first segment, the second segment, and/or the at least one additional segment are replaced by a multiple cloning site.

Also provided herein is a vector comprising any one of the vector designs of FIG. 1A or FIG. 1B. Further provided herein is a composition comprising vectors comprising the 3-vector design or the 2-vector design of FIG. 1A or FIG. 1B.

Yet other aspects herein provide a kit comprising vectors that comprise the 3-vector design or the 2-vector design of FIG. 1A or FIG. 1B, wherein the Exon 1 and Exon 2 are each replaced by a multiple cloning site.

Further aspects of the present disclosure provide a nucleic acid vector comprising, in a 5′ to 3′ orientation, a coding region, a splice donor site, a recombination site, and optionally a 5′ LTR and a 3′ LTR. In some embodiments, the vector further comprises a promoter upstream from and operably linked to the coding region, and optionally further comprising 5′ LTR and a 3′ LTR. In some embodiments, the vector further comprises a recombination site upstream from the coding region. Yet other aspects provide a nucleic acid vector comprising, in a 5′ to 3′ orientation, a recombination site, a splice acceptor site, a coding region, optionally a post-transcriptional regulator element, and optionally a 5′ LTR and a 3′ LTR. In some embodiments, the vector further comprises a promoter, a recombination site, a coding region that encodes a site-specific nucleic acid-rearranging enzyme (e.g., as site-specific recombinase), and optionally a post-transcriptional regulator element, wherein the promoter is operably linked to the coding region that encodes a site-specific nucleic acid-rearranging enzyme. Still other aspects provide a nucleic acid vector comprising, in a 5′ to 3′ orientation, a promoter operably linked to a coding region that encodes a site-specific nucleic acid-rearranging enzyme (e.g., as site-specific recombinase), a post-transcriptional regulator element, optionally a 5′ LTR and a 3′ LTR, and optionally a recombination site upstream from the coding region and another recombination site downstream from the coding region.

Some aspects of the present disclosure provide method comprising delivering to a cell (a) a first vector comprising a first segment of a gene of interest and a first recombination site, (b) a second vector comprising a second segment of the gene of interest and a second recombination site, (c) and a cognate site-specific recombinase or a nucleic acid encoding a cognate site-specific recombinase. In some embodiments, (c) is a nucleic acid encoding a cognate site-specific recombinase.

In some embodiments, the nucleic acid encoding a cognate site-specific recombinase is delivered on the first or second vector. In other embodiments, the nucleic acid encoding a cognate site-specific recombinase is delivered on a third vector.

Other aspects of the present disclosure provide a method comprising delivering to a cell (a) a first vector comprising a first nucleic acid comprising, optionally in a 5′ to 3′ orientation, a first promoter operably linked to a first segment of a gene of interest, a splice donor site, and a first recombination site, wherein the first nucleic acid is flanked by a first pair inverted terminal repeat sequences (ITRs)/long terminal repeats (LTRs), (b) a second vector comprising a second nucleic acid comprising, optionally in a 5′ to 3′ orientation, a second recombination site, a splice acceptor site, a second segment of the gene of interest, and a post-transcriptional regulator element, optionally WPRE, wherein the second nucleic acid is flanked by a second pair of ITR/LTR sequences, and (c) a third vector comprising a third nucleic acid comprising a second promoter operably linked to a nucleotide sequence encoding a cognate site-specific recombinase and a post-transcriptional regulator element, optionally WPRE, wherein the third nucleic acid is flanked by a second pair of ITR/LTR sequences.

In some embodiments, the cognate site-specific recombinase catalyzes a recombination event to join the first segment to the second segment.

In some embodiments, the vector is a plasmid.

In some embodiments, the vector is a viral vector. In some embodiments, wherein the viral vector is selected from the group consisting of adeno-associated viral vectors, adenoviral vectors, lentiviral vectors, and retroviral vectors. In some embodiments, the viral vector is an adeno-associated viral (AAV) vector, optionally an AAV2 vector.

In some embodiments, the site-specific recombinase is a serine recombinase. In some embodiments, the serine recombinase is selected from the group consisting of Bxb1 recombinase, TP901-1 recombinase, PhiC31 recombinase, TG1 recombinase, and PhiRv1 recombinase. In some embodiments, the serine recombinase is a Bxb1 recombinase.

In some embodiments, the site-specific recombinase is a tyrosine recombinase. In some embodiments, the tyrosine recombinase is selected from the group consisting of Cre recombinase, Flp recombinase, XerC/D recombinase, and XerA recombinase. In some embodiments, the tyrosine recombinase is Cre recombinase.

In some embodiments, the first segment is a first exon of the gene of interest, and the second segment is a second exon of the gene of interest. In some embodiments, the gene of interest is a therapeutic gene of interest and/or encodes a therapeutic protein. In some embodiments, the gene of interest encodes a Cas protein, optionally a Cas9 or Cas12a protein, optionally fused to a transcriptional activator, a transcriptional repressor, or a deaminase.

Also provided herein, in some aspects, is a composition, cell, or kit comprising (a) a first vector comprising a first segment of a gene of interest and a first recombination site, (b) a second vector comprising a second segment of the gene of interest and a second recombination site, (c) and a cognate site-specific recombinase or a nucleic acid encoding a cognate site-specific recombinase.

Further provided herein, in some aspects, is a composition, cell, or kit comprising (a) a first vector comprising a first nucleic acid comprising, optionally in a 5′ to 3′ orientation, a first promoter operably linked to a first segment of a gene of interest, a splice donor site, and a first recombination site, wherein the first nucleic acid is flanked by a first pair ITR/LTR sequences, (b) a second vector comprising a second nucleic acid comprising, optionally in a 5′ to 3′ orientation, a second recombination site, a splice acceptor site, a second segment of the gene of interest, and a post-transcriptional regulator element, optionally WPRE, wherein the second nucleic acid is flanked by a second pair of ITR/LTR sequences, and (c) a third vector comprising a third nucleic acid comprising a second promoter operably linked to a nucleotide sequence encoding a cognate site-specific recombinase and a post-transcriptional regulator element, optionally WPRE, wherein the third nucleic acid is flanked by a second pair of ITR/LTR sequences.

### BRIEF DESCRIPTION OF THE DRAWINGS

FIG. 1A: Assembly of two AAV viral payloads using site-specific recombinases (SSR). (1) AAV viral vectors showing placement of recombination sites (RS). 3-vector design supplies SSR on a separate virus than the assembled cargo. 2-vector system has bxb1 contained on one of the same virus as assembled cargo. (2) SSR catalyzes ligation of vectors together. (3) Transcription and RNA-splicing yields gene product. FIG. 1B: Assembly of two AAV viral payloads using site-specific recombinases (SSR) containing a protective switch, whereby a recombination site is placed between the promoter and SSR, resulting in promoter cleavage after one recombination event, thus preventing uncontrolled expression of SSR.

FIG. 2: Sanger sequencing confirmation of joining of two AAV2 vectors by Bxb1 integrase using 3-vector design strategy. Sanger sequencing results show formation of an attL post-recombination site from Bxb1-mediated assembly of two mKate exons from two AAV2 viruses in living mammalian cells. SEQ ID NOs: 177-179 are indicated.

FIG. 3: Flow cytometric results show expression of assembled mKate fluorescent protein gene from two AAV2 vectors by bxb1 integrase using 2-vector design strategy. Flow cytometric results show expression of mKate fluorescent protein from bxb1-mediated assembly of two mKate exons from two AAV2 viruses in living mammalian cells. Blue dots indicate non-treated cells and red dots indicate those treated with respective conditions. Bxb1(S10A) is a serine to alanine mutation at amino acid residue 10 that deactivates bxb1 site-specific recombination.

FIGS. 4A-4B: In vitro assembly of DNA by Cre recombinase is shown. FIG. 4A: Schematic showing production of two double-stranded DNA fragments containing lox sites using PCR with fluorescently labelled primers (Cy5 or IRD800). FIG. 4B: Results after fragments were incubated together (equimolar and 25 ng of Cy5 left fragment) at 37° C. with (15 U) or without Cre recombinase protein in 1×Cre Reaction Buffer (New England Biolabs) for given amounts of time are shown. Upon completion, reactions were halted with Proteinase K or through 70° C. heat inactivation (indicated with *). EtBr indicates ethidium bromide fluorescence from a 2% ethidium bromide agarose gel.

FIGS. 5A-5C: Assembly of plasmid DNA by Cre recombinase in living mammalian cells is shown. FIG. 5A: A schematic depicting the two AAV ITR plasmids used to produce an assembled ITR plasmid is shown. The left ITR plasmid (LP) was constructed with a lox71 sequence downstream of a human EF1 (hEF1) promoter. The right ITR plasmid (RP) was constructed with a lox66 site upstream of a GFP-WPRE sequence. Primer sites are indicated with half arrows. FIG. 5B: Flow cytometry was performed on the cells 48 hours post-transfection with the plasmids in FIG. 5A in different combinations along with plasmids containing the pCAG promoter driving Cre or Flp recombinases in human embryonic kidney cells (HEK293T). All transfections also included a pCAG-BFP transfection marker plasmid. GFP mean fluorescence intensity (MFI) was determined on single cells containing BFP fluorescence. A.U. indicates arbitrary units. Error bars indicate standard error of the mean over n=3 transfected cell cultures. FIG. 5C: Plasmid DNA was isolated and PCRs were performed using primer sites indicated in FIG. 5A. A 480 bp band was expected if assembly was successful. PCR results are shown.

### DETAILED DESCRIPTION

### Vectors

A vector used as provided herein, in some embodiments, is a viral vector. In some embodiments, a viral vector is not a naturally occurring viral vector. The viral vector may be from adeno-associated virus (AAV), adenovirus, herpes simplex virus, lentiviral, retrovirus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, or human parvovirus B 19. Other viral vectors are encompassed by the present disclosure.

In some embodiments, a viral vector is an AAV vector. AAV is a small, non-enveloped virus that packages a single-stranded linear DNA genome that is approximately 5 kb long and has been adapted for use as a gene transfer vehicle (Samulski, R J et al., Annu Rev Virol. 2014; 1(1):427-51). The coding regions of AAV are flanked by inverted terminal repeats (ITRs), which act as the origins for DNA replication and serve as the primary packaging signal (McLaughlin, S K et al. Virol. 1988; 62(6): 1963-73; Hauswirth, W W et al. 1977; 78(2):488-99). Thus, an AAV vector typically includes ITR sequences. Both positive and negative strands are packaged into virions equally well and capable of infection (Zhong, L et al. Mol Ther. 2008; 16(2):290-5; Zhou, X et al. Mol Ther. 2008; 16(3):494-9; Samulski, R J et al. Virol. 1987; 61(10):3096-101). In addition, a small deletion in one of the two ITRs allows packaging of self-complementary vectors, in which the genome self-anneals after viral uncoating. This results in more efficient transduction of cells but reduces the coding capacity by half (McCarty, D M et al. Mol Ther. 2008; 16(10): 1648-56; McCarty, D M et al. Gene Ther. 2001; 8(16): 1248-54).

In some embodiments, a vector comprises a nucleotide sequence encoding a nucleic acid sequence operably linked to a promoter (promoter sequence). In some embodiments, the promoter is an inducible promoter (e.g., comprising a tetracycline-regulated sequence). Inducible promoters enable, for example, temporal and/or spatial control of gene expression.

A promoter control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof. A promoter drives expression or drives transcription of the nucleic acid sequence that it regulates. Herein, a promoter is considered to be operably linked when it is in a correct functional location and orientation in relation to a nucleic acid sequence it regulates to control (“drive”) transcriptional initiation and/or expression of that sequence.

An inducible promoter is one that is characterized by initiating or enhancing transcriptional activity when in the presence of, influenced by or contacted by an inducing agent. An inducing agent may be endogenous or a normally exogenous condition, compound or protein that contacts an engineered nucleic acid in such a way as to be active in inducing transcriptional activity from the inducible promoter.

Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

The vectors of the present disclosure may be generated using standard molecular cloning methods (see, e.g., Current Protocols in Molecular Biology, Ausubel, F. M., et al., New York: John Wiley & Sons, 2006; Molecular Cloning: A Laboratory Manual, Green, M. R. and Sambrook J., New York: Cold Spring Harbor Laboratory Press, 2012; Gibson, D. G., et al., Nature Methods 6(5):343-345 (2009), the teachings of which relating to molecular cloning are herein incorporated by reference).

### Payloads

The methods and compositions of the present disclosure may be used, for example, to deliver to a cell a payload. A payload, herein, can be any polynucleotide (nucleic acid) of interest. In some embodiments, a payload is a nucleic acid that encodes a molecule of interest or a portion of a molecule of interest, such as, for example, a polypeptide (e.g., protein) of interest. Thus, in some embodiments, a payload is a gene of interest or a segment of a gene of interest.

Vectors described herein are limited in size capacity, which prevents delivery of large nucleic acid sequences. Thus, these large nucleic acid sequences may be divided among two or more vectors, delivered to a cell, and then assembled within the cell. As described above, AAV, for example, has a capacity of only 4.7 kb. AAV vectors may be used as described herein to deliver nucleic acids that are larger than 4.7 kb by dividing the nucleic acid into two or more segments, each segment having a size of smaller than 4.7 kb. Each segment can be delivered to a cell on an independent AAV vector. Other viral vectors may be used in a similar manner, dividing the nucleic acid into segments, guided by size capacity of the vector. Thus, a single gene, for example, may be delivered to a cell by delivering multiple vectors, each payload of the vector being a segment of the gene.

### Therapeutic Molecules

In some embodiments, the methods and compositions of the present disclosure are used to deliver a therapeutic gene to a cell. For example, a first second and a second segment described herein may together (when joined and transcribed/translated together) form a therapeutic gene or encode a therapeutic protein. Table 1 provides examples of therapeutic genes/proteins and their related diseases.

| | | Implicated | Coding |
|---|---|---|---|
| Gene | Description | disease | sequence (kb) |
| USH2A | Usherin | Usher | 15.606 |
| | | syndrome IIA, | |
| | | retinitis | |
| | | pigmentosa | |
| PKD1 | Polycystin | Polycystic | 12.909 |
| | | kidney | |
| | | disease | |
| ALMS1 | Alstrom syndrome | Alstrom | 12.504 |
| | protein 1 | syndrome | |
| PKHD1 | Fibrocystin | Polycystic | 12.222 |
| | | kidney | |
| | | disease | |
| VPS13B | Vacuolar protein | Cohen | 12.066 |
| | sorting- | syndrome | |
| | associated | | |
| | protein 13B | | |
| DMD | Dystrophin | Muscular | 11.055 |
| | | dystrophy | |
| HD | Huntingtin | Huntington | 9.426 |
| | | disease | |
| COL7A1 | Collagen alpha-1 | Recessive | 8.832 |
| | (VII) chain | dystrophic | |
| | | epidermolysis | |
| | | bullosa | |
| | | (RDEB) | |
| CEP290 | Centrosomal | Bardet-Biedl, | 7.437 |
| | protein of | Joubert, | |
| | 290 kDa | Meckel, and | |
| | | Senior- | |
| | | Løken | |
| | | ciliopathies | |
| ABCA4 | Retinal-specific | Stargardt | 6.819 |
| | ATP- | disease | |
| | binding cassette | | |
| | transporter | | |
| MYO7A | Unconventional | Usher | 6.645 |
| | myosin-VIIa | syndrome 1B | |
| NHS | Nance-Horan | Nance-Horan | 4.953 |
| | syndrome | syndrome | |
| | protein | | |
| COL17A1 | Collagen alpha-1 | Epidermolysis | 4.491 |
| | (XVII) | bullosa | |
| | chain | | |
| CFTR | Cystic fibrosis | Cystic fibrosis | 4.440 |
| | transmembrane | | |
| | conductance | | |
| | regulator | | |

The size of the therapeutic gene, other gene of interest, or other nucleic acid of interest may vary. In some embodiments, the nucleic acid (e.g., gene) has a size of at least 4 kilobases (kb). For example, the gene may have a size of at least 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 kb. In some embodiments, the nucleic acid (e.g., therapeutic gene or other gene of interest) has a size of 4-20, 4-19, 4-18, 4-17, 4-16, 4-15, 4-14, 4-13, 4-12, 4-11, 4-10, 4-9, 4-8, 4-7, 4-6, or 4-5 kb. In some embodiments, the nucleic acid (e.g., therapeutic gene or other gene of interest) has a size of 5-20, 5-19, 5-18, 5-17, 5-16, 5-15, 5-14, 5-13, 5-12, 5-11, 5-10, 5-9, 5-8, 5-7, or 5-6 kb. In some embodiments, the nucleic acid (e.g., therapeutic gene or other gene of interest) has a size of 6-20, 6-19, 6-18, 6-17, 6-16, 6-15, 6-14, 6-13, 6-12, 6-11, 6-10, 6-9, 6-8, or 6-7 kb. In some embodiments, the nucleic acid (e.g., therapeutic gene or other gene of interest) has a size of 7-20, 7-19, 7-18, 7-17, 7-16, 7-15, 7-14, 7-13, 7-12, 7-11, 7-10, 7-9, or 7-8 kb. In some embodiments, the nucleic acid (e.g., therapeutic gene or other gene of interest) has a size of 8-20, 8-19, 8-18, 8-17, 8-16, 8-15, 8-14, 8-13, 8-12, 8-11, 8-10, or 8-9 kb. In some embodiments, the nucleic acid (e.g., therapeutic gene or other gene of interest) has a size of 9-20, 9-19, 9-18, 9-17, 9-16, 9-15, 9-14, 9-13, 9-12, 9-11, or 9-10 kb. In some embodiments, the nucleic acid (e.g., therapeutic gene or other gene of interest) has a size of 10-20, 10-19, 10-18, 10-17, 10-16, 10-15, 10-14, 10-13, 10-12, or 10-11 kb.

The size of a nucleic acid segment forming part of a gene or encoding part of a protein may vary. Any of the nucleic acid segments (e.g., a first segment and/or a second segment) may have a size of 0.5 kb to 10 kb. Larger segments are also contemplated herein. In some embodiments, a first and/or second segment has a size of 0.5 kb, 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb, or 10 kb. In some embodiments, a first and/or second segment has a size of 1-10 kb, 2-10 kb, 3-10 kb, 4-10 kb, 5-10 kb, 6-10 kb, 7-10 kb, 8-10 kb, or 9-10 kb.

### Gene Editing Molecules

In some embodiments, the methods and compositions of the present disclosure are used to deliver nucleic acid molecules that collectively encode a protein (e.g., enzyme) used in gene editing. For example, the methods and compositions of the present disclosure may be used to deliver nucleic acid molecules that collectively encode Cas9 protein (or another Cas protein, such as Cas12a protein) and/or guide RNA (gRNA). Cas9 protein is from Streptococcus pyogenes and is a 1367 amino acid (4.101 kb) RNA-guided DNA endonuclease that has been adopted for making DNA edits in genomes of living human cells. Other examples include larger Cas9 variations which have been fused with additional sequences, such as transcription activators (e.g. VP64, p65), transcription repressors (e.g., KRAB), and deaminases for further functionality; these additional sequences further complicate and prevent the packaging into a single AAV vector, for example.

### Site-Specific Nucleic Acid-Rearranging Enzymes

A site-specific nucleic acid-rearranging enzyme is any enzyme that can catalyze the reciprocal exchange of nucleic acid between define sites, referred to herein as recombination sites.

In some embodiments, the site-specific enzyme is selected from the group consisting of site-specific recombinases, transposases, and retrotransposases.

### Site-Specific Recombinases

In some embodiments, the site-specific enzyme is a site-specific recombinase. Site-specific recombinases (SSRs) can rearrange nucleic acid (e.g., DNA) segments by recognizing and binding to short nucleic acid sequences (recombination sites), at which they cleave the nucleic acid backbone, exchange the two nucleic acids (e.g., DNA helices) involved and rejoin the nucleic acid strands. Based on amino acid sequence homology and mechanistic relatedness, most site-specific recombinases are grouped into one of two families: the tyrosine recombinase family or the serine recombinase family. The names stem from the conserved nucleophilic amino acid residue that they use to attack the DNA and which becomes covalently linked to it during strand exchange. Non-limiting examples of site-specific recombinases are described herein and include, Flp, KD, B2, B3, R, Cre, VCre, SCre, Vika, Dre, λ-Int, HK022, φC31, Bxb1, Gin, and Tn3. Table 2 provides non-limiting examples of site-specific recombinases and their corresponding recombination sites.

TABLE 2
Example Site-Specific Recombinases*

| | | | | | SEQ |
|---|---|---|---|---|---|
| | | Classifi- | Target | | ID |
| Recombinase | Origin | cation | site | Target sequence | NO: |
| Flp | S. cerevisiae | Tyrosine | FRT | 5′- | 1 |
| | | | | GAAGTTCCTATTCTCTAGA | |
| | | | | AAGTATAGGAACTTC-3′ | |
| KD | K. | Tyrosine | KDRT | 5′- | 2 |
| | drosophilarum | | | AAACGATATCAGACATTT | |
| | | | | GTCTGATAATGCTTCATTA | |
| | | | | TCAGACAAATGTCTGATAT | |
| | | | | CGTTT-3′ | |
| B2 | Z. bailii | Tyrosine | H2RT | 5′- | 3 |
| | | | | GAGTTTCATTAAGGAATA | |
| | | | | ACTAATTCCCTAATGAAAC | |
| | | | | TC-3′ | |
| B3 | Z. bisporus | Tyrosine | B3RT | 5′- | 4 |
| | | | | GGTTGCTTAAGAATAAGT | |
| | | | | AATT′CTTAAGCAACC-3′ | |
| R | Z. rouxii | Tyrosine | RSRT | 5′- | 5 |
| | | | | TTGATGAAAGAATAACGT | |
| | | | | ATTCTTTCATCAA-3′ | |
| Cre | Phage P1 | Tyrosine | loxP | 5′- | 6 |
| | | | | ATAACTTCGTATAGCATAC | |
| | | | | ATTATACGAAGTTAT-3′ | |
| VCre | Vibrio sp. | Tyrosine | VloxP | 5′- | 7 |
| | | | | TCAATTTCTGAGAACTGTC | |
| | | | | ATTCTCGGAAATTGA-3′ | |
| SCre | Shewattella | Tyrosine | SloxP | 5′- | 8 |
| | sp. | | | CTCGTGTCCGATAACTGTA | |
| | | | | ATTATCGGACATGAT-3′ | |
| Vika | V. | Tyrosine | vox | 5′- | 9 |
| | coralliilyticus | | | AATAGGTCTGAGAACGCC | |
| | | | | CATTCTCAGACGTATT-3′ | |
| Dre | Bacteriophage | Tyrosine | rox | 5′- | 10 |
| | D6 | | | TAACTTTAAATAATGCCAA | |
| | | | | TTATTTAAAGTTA-3′ | |
| λ-nt | Phage λ | Tyrosine | attP | 5′- | 11 |
| | | | | CAGCTTTTTTATACTAAGT | |
| | | | | TG-3′ | |
| | | | attB | 5′- | 12 |
| | | | | CTGCTTTTTTATACTAACT | |
| | | | | TG-3′ | |
| HK022 | Phage HK022 | Tyrosine | attP | 5′- | 13 |
| | | | | ATCCTTTAGGTGAATAAGT | |
| | | | | TG-3′ | |
| | | | attB | 5′- | 14 |
| | | | | GCACTTTAGGTGAAAAAG | |
| | | | | GTT-3′ | |
| φC31 | Phage φC31 | Serine | attP | 5′- | 15 |
| | | | | CCCCAACTGGGGTAACCTT | |
| | | | | TGAGTTCTCTCAGTTGGGG | |
| | | | | -3′ | |
| | | | attB | 5′- | 16 |
| | | | | GTGCCAGGGCGTGCCCTTG | |
| | | | | GGCTCCCCGGGCGCG-3′ | |
| Bxb1 | Phage Bxb1 | Serine | attP | 5′- | 17 |
| | | | | GGTTTGTCTGGTCAACCAC | |
| | | | | CGCGGTCTCAGTGGTGTAC | |
| | | | | GGTACAAACC-3′ | |
| | | | attB | 5′- | 18 |
| | | | | GGCTTGTCGACGACGGCG | |
| | | | | GTCTCCGTCGTCAGGATCA | |
| | | | | T-3′ | |
| Gin | Phage Mu | Serine | gix | 5′- | 19 |
| | | | | TTATCCAAAACCTCGGTTT | |
| | | | | ACAGGAA-3′ | |
| Tn3 | E. coli | Serine | res | 5′- | 20 |
| | | | site | CGTTCGAAATATTATAAAT | |
| | | | 1 | TATCAGACA-3′ | |

*Gaj T et al. Biotechnol Bioeng. 2014; 111(1): 1-15, incorporated herein by reference

Non-limiting examples of tyrosine recombinase family molecules that may be used as a site-specific recombinase include Cre, Flp, XerC/D, XerA, Lambda, P2, HK022, FimB, FimE, HbiF, Rci, Cryptons, DIRS, Ngaro, PAT, Tec, Tn916, CTnDOT, topoisomerase IB, telomere resolvases, Y1-transposases of IS200/IS605 (e.g., IS608 TnpA, ISDra2), ISC (e.g. IscA), Helitrons, IS91, AAV Rep78, TrwC relaxase, MrpA, XerH, XerS, DAI, SSV, PhiCh1, pNOB, pTN3, IntC, IntG, IntI, and SNJ2 recombinases.

Non-limiting examples of serine recombinase family molecules that may be used as a site-specific recombinase include Tn3, gamma-delta, Gin, Hin, Gin, Hin, Bxb1, TP901-1, PhiC31, TG1, PhiRv1, and C.IS607-like serine transposase.

Other site-specific recombinases may be used. For example, Yang L et al. provides phage integrases that may be used in accordance with the present disclosure (see, e.g., Supplementary Table 1 of Yang Let al. Nat Methods. 2014; 11(12): 1261-1266, incorporated herein by reference). Table 3 below provides additional examples of site-specific recombinases that may be used as provided herein.

In some embodiments, a recombination site is positioned between a promoter and a coding region for a site-specific recombinase, which results in promoter cleavage after one recombination event, thus preventing uncontrolled expression of the site-specific recombinase. The design of this “protective” switch can be used to address any off-target genome effects due to potential high copy number expression and prolonged exposure of the site-specific recombinase.

### Transposases and Retrotransposases

In some embodiments, the site-specific enzyme is transposase. A transposase is an enzyme that binds to the end of a transposon and catalyzes its movement to another part of the genome by a cut and paste mechanism or a replicative transposition mechanism. Most transposases include a DDE motif (herein referred to as DDS transposases), which is the active site that catalyzes the movement of the transposon. Aspartate-97, Aspartate-188, and Glutamate-326 make up the active site, which is a triad of acidic residues.

In some embodiments, the site-specific enzyme is a retrotransposase. Retrotransposons are genetic elements that can amplify themselves in a genome and are ubiquitous components of the DNA of many eukaryotic organisms. These DNA sequences are first transcribed into RNA, then converted back into identical DNA sequences using reverse transcription, and these sequences are then inserted into the genome at target sites. In some embodiments, the retrotransposase is a long-terminal repeat (LTR) transposase. LTR retrotransposons have direct LTRs that range from ˜100 bp to over 5 kb in size. LTR retrotransposons are further sub-classified into the Ty1-copia-like (Pseudoviridae), Ty3-gypsy-like (Metaviridae), and BEL-Pao-like groups based on both their degree of sequence similarity and the order of encoded gene products. In some embodiments, the retrotransposase comprises a DDE motif and a LTR (referred to herein as a DDE LTR-retrotransposase). In some embodiments, the retrotransposase is a target-primed retrotransposases, such as a long interspersed nuclear element (LINE). retrotransposase.

### Cells

The methods herein may be used to deliver payloads to any cell. In some embodiments, the cell is a cell of a model organism, such as mouse, rat, or monkey. In some embodiments, the cell is a mammalian cell. The mammalian cell may be, for example, a human cell.

### EXAMPLES

### Example 1

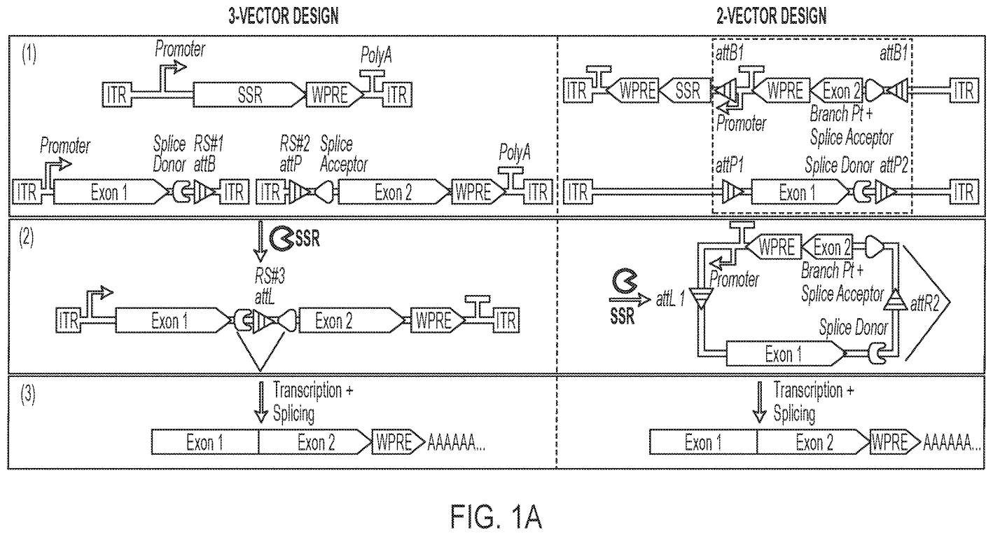

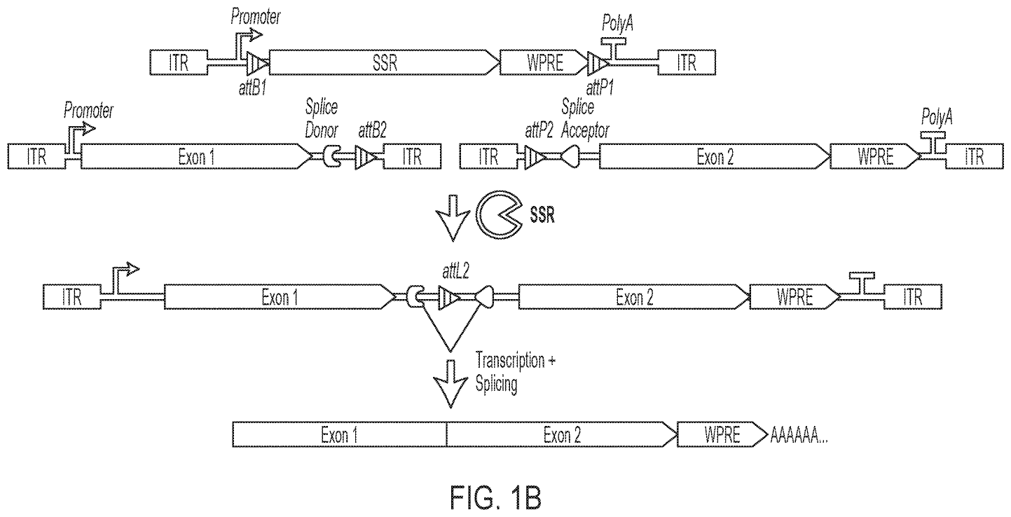

First, nucleic acid vectors are generated. Each vector that is delivered and assembled together contains a recombination site (RS) sequence of the specific site-specific recombinase (SSR) that is used. Long genes that cannot be contained in a single vector are designed into multiple nucleic acid segments to be split among multiple vectors (FIG. 1). Some SSRs have the capacity to join more than two nucleic acid molecules together in a site-specific manner through design of central spacer sequences (e.g., 6 base pair (bp) central region of Cre loxP; 2 bp central region of Bxb1 attB/P sequences). Such RSs are designed in a fashion to connect nucleic acids in a desired order. Since a single RS sequence remains after a recombination event, this “scar” sequence can be transcribed and translated within a gene product if it is contained within an exonic region. If that is not desired, RNA splicing donor, branch point, and acceptor sequences (natural or synthetic) can be placed strategically, such that post-recombined RSs are contained within intronic regions (e.g., splice donor upstream of RS and branch point+splice acceptor downstream of RS); thereby removing RS from mRNA and the translated gene product. Finally, vectors are packaged and delivered to cells along with SSR. While an SSR can be introduced to cells in a similar fashion as the RS-containing sequences, it can be delivered through other means, such as in a purified protein formulation.

### Example 2

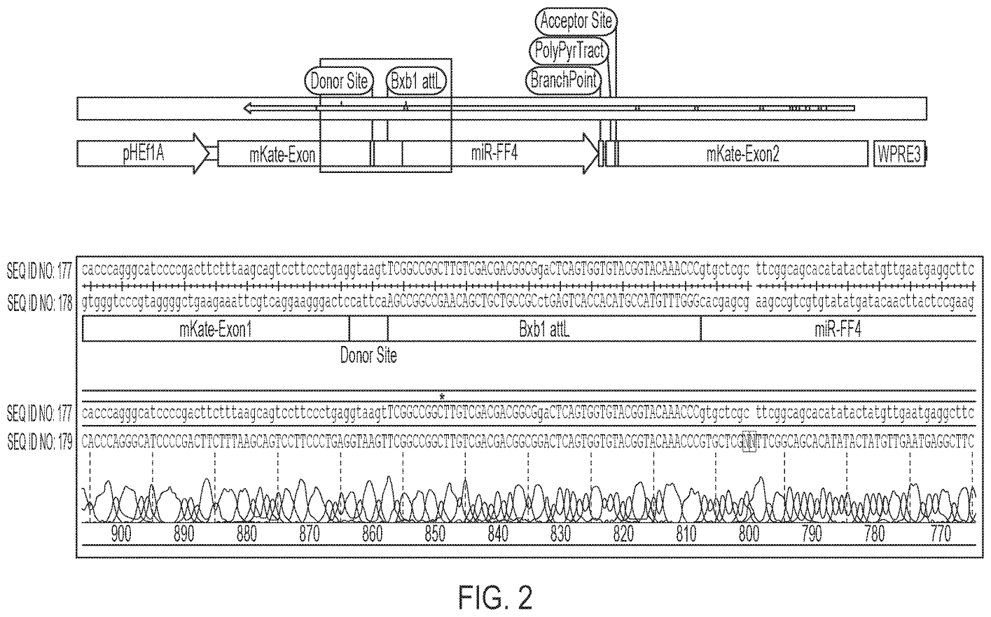

The methods described herein have been demonstrated in living human embryonic kidney (HEK293T) cells. Sanger sequencing confirmed joining of two AAV2 vectors by Bxb1 integrase using a 3-vector design strategy (FIG. 2). Sanger sequencing results show formation of an attL post-recombination site from Bxb1-mediated assembly of two mKate exons from two AAV2 viruses in living mammalian cells (FIG. 2).

### Example 3

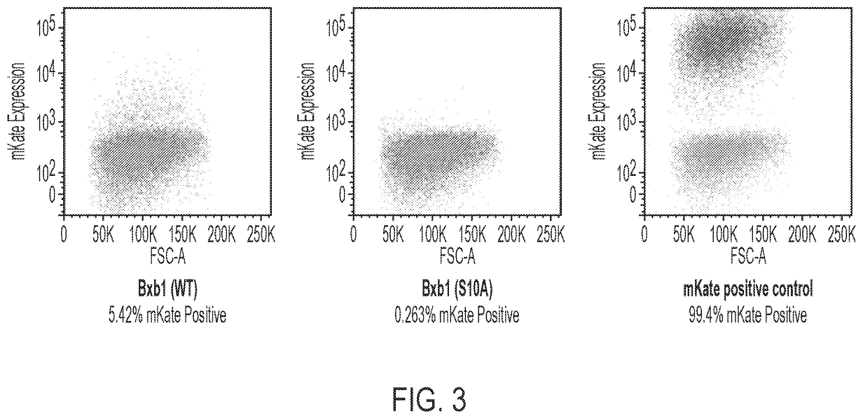

Flow cytometric results showed expression of assembled mKate fluorescent protein gene from two AAV2 vectors by Bxb1 integrase using a 2-vector design strategy (FIG. 3). Flow cytometric results show expression of mKate fluorescent protein from Bxb1-mediated assembly of two mKate exons from two AAV2 viruses in living mammalian cells (FIG. 3).

### Example 4

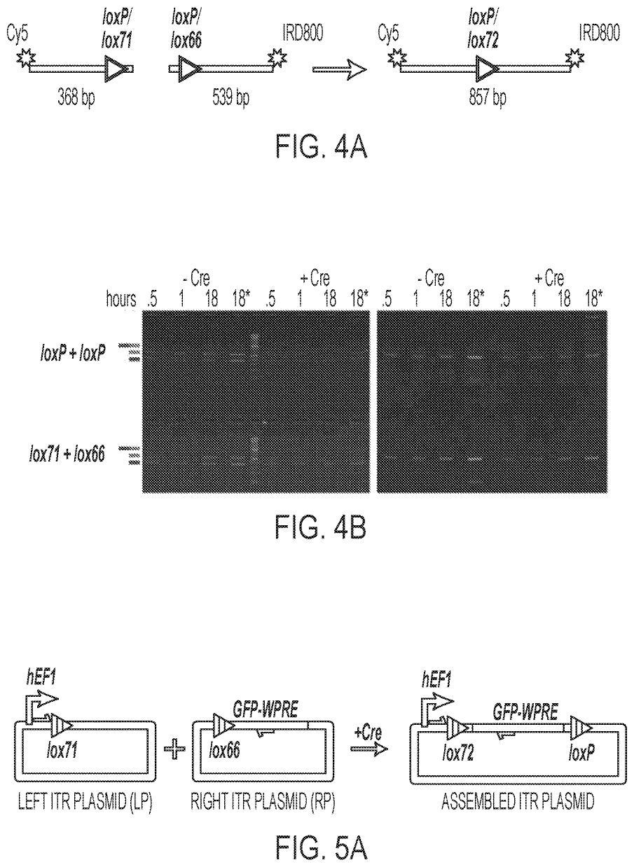

Cre-mediated assembly of two DNA fragments was tested in vitro. Two double-stranded DNA fragments containing lox sites were created by PCR using fluorescently labelled primers (Cy5 or IRD800) (FIG. 4A). Fragments were incubated together (equimolar and 25 ng of Cy5 left fragment) at 37° C. with (15 U) or without Cre recombinase protein in 1×Cre Reaction Buffer (NEW ENGLAND BIOLABS®) for given amounts of time. Upon completion, reactions were halted with Proteinase K or through 70° C. heat inactivation (indicated with * in FIG. 4B). PCR reactions were found to have IRD800 fluorescence for reactions with IRD800 primers (data not shown).

### Example 5

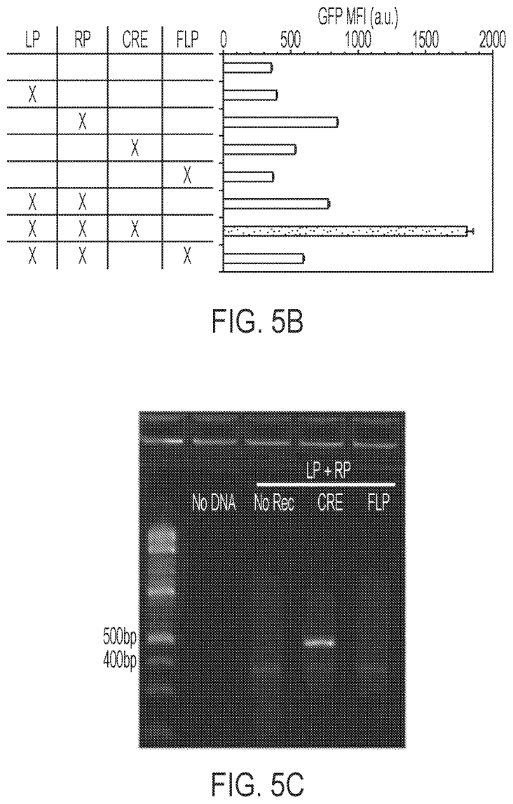

The assembly of plasmid DNA by Cre recombinase was tested in living mammalian cells. As shown in FIG. 5A, two AAV ITR plasmids were constructed. The left ITR plasmid (LP) was constructed with a lox71 sequence downstream of a human EF1 (hEF1) promoter. The right ITR plasmid (RP) was constructed with a lox66 site upstream of a GFP-WPRE sequence. These plasmids were transiently transfected in different combinations along with plasmids containing the pCAG promoter driving Cre or Flp recombinases in human embryonic kidney cells (HEK293T) using polyethylenimine. All transfections also included a pCAG-BFP transfection marker plasmid.

Flow cytometry was performed on the cells 48 hours post-transfection and GFP mean fluorescence intensity (MFI) was determined on single cells containing BFP fluorescence. As shown in FIG. 5B, successful assembly of the ITR plasmid was detected in cells transfected with the LP, RP, and the plasmid with the pCAG promoter driving Cre recombinase expression.

Plasmid DNA was isolated and PCR was performed using primer sites indicated in FIG. 5A. A 480 bp band was expected if assembly was successful. As shown in FIG. 5C, the assembled ITR plasmid was detected in plasmid DNA isolated from cells that were transfected with the LP, RP, and the plasmid with the pCAG promoter driving Cre recombinase expression. PCR products were purified and Sanger sequencing confirmed the formation of the lox72 site (data not shown).

TABLE 3
Additional Examples of SSRs

| | Recombinase | | | | SEQ |
|---|---|---|---|---|---|
| NCBI | name/ | | Protein | | ID |
| identifier: | identifier: | Protein sequence: | (aa): | Type: | NO: |
| CAL92453 | hypothetical | mtdqpgnaidrnvercqecdemseadaeai | 405 | BJ1 | 21 |
| | protein | ldahrqmellgasrlskshhsdvlmravkm | | | |
| | [Archaeal BJ1 | arevgglanaleereateeivrwiqrtydn | | | |
| | virus] | eetnrdyrkclrafgrhatrseeppdsiaw | | | |
| | | vpagysntydpapdpgemfrwqkhvkpmvd | | | |
| | | assnvrdealvalcwdlgprtselhelqvs | | | |
| | | niteadyglrvtiengkngsrsptivkatp | | | |
| | | yvrdwlerhpgdrddylwsrlnspkrvsrn | | | |
| | | ylrdtlkrlasnaamdppatptptqlrkss | | | |
| | | asylarqnvnqtfiedhhgwvrgsdkaary | | | |
| | | vavfddssddaiasahgvdvditddtpsmq | | | |
| | | ecvrcdelnepdrsrcrrcgyaltqeavet | | | |
| | | eetreerfnkqlamldkenamrlvevmdal | | | |
| | | ddpevlaaldevasr | | | |
| WP_004217472 | integrase | mtdadpreevdtlrdrlrssgedaryvqfe | 453 | BJ1 | 22 |
| | [Natrialba | adrrhllkfsdnirlvpseigdhrhlkllr | | | |
| | magadii] | hccrmaalvppptvedfkdndeaadagivd | | | |
| | | eddvddlleehgllgltleyraaaegvvrw | | | |
| | | ineeyanehtnqdyrtalrsfgryrlkrde | | | |
| | | ppesltwiptgtsndfdpvpserdllthdd | | | |
| | | vramieegsrnprdkallavqfeaglrgge | | | |
| | | lydvrvgdvfdgehsvglhvdgkegersvh | | | |
| | | litsvpylqqwltshpapdddqawlwskls | | | |
| | | saerpsyatflnyfknaaarvdvtkdvtpt | | | |
| | | nfrksntrwlilqnfstariedrqgrkrgs | | | |
| | | ehtarymarfgeesnerayaqlhgldvean | | | |
| | | eteevappvpcprcgedtpsdrdfcihchq | | | |
| | | sldfeakelldevrevldnrsieaedpedr | | | |
| | | refvsarrdeekphvmdkddlhefasslsa | | | |
| | | ed | | | |
| WP_004972504 | Phage | mpsdpkqsvatlrkklrngtrggcdrdrel | 435 | BJ1 | 23 |
| | integrase | lldfsdelrllredyghyrhekllrhnvri | | | |
| | [Haloferax | senaetclhetlvrerdgdaddeetfydak | | | |
| | gibbonsii] | daakvvvrwihgtydiedgsqetnrdyrva | | | |
| | | frlfakhvtrgddipdthswistktsrdyq | | | |
| | | pepdeadmldlerdvepmieaarnprdkal | | | |
| | | ialqfeggfrggelydmrveditdgkhslk | | | |
| | | vrvdgkrgehdvhlivavpyvkrwlaehpg | | | |
| | | dhddylwtklteperfsytrflqcfkaagk | | | |
| | | raeirkpvtptnfiksnaywlstreksqaf | | | |
| | | iedrqgrargspvisryvakfsgetqeiqy | | | |
| | | aamhgleavetetkelapvtcprceketpr | | | |
| | | ergfcihcnqsldieskelldrigtaiddk | | | |
| | | vveaddadtrrdllrarrtlderpammdte | | | |
| | | elhelasrfslsdea | | | |
| WP_006672730 | integrase | mattprkridslrdraetggdigdrdrell | 403 | BJ1 | 24 |
| | [Halobiforma | lefsdtldllaqeysdhrhekllrhcvima | | | |
| | nitratireducens] | eeledntiaaaldnrdatetivawinrnyd | | | |
| | | neetnrdyrsairvfakrvtdgsecpptvd | | | |
| | | wvptgtsrnydpspdpremlkweddavpmi | | | |
| | | decfnardaamialqfdaglrggefksltv | | | |
| | | gdiqdhdhglqvtvegkqgrrtimlipsvp | | | |
| | | yvnrwlddhpdrddpdaplwskitkvegis | | | |
| | | drmvskvfdeaagragvekpvtltnfrkss | | | |
| | | aaflasrnlnqahieehhgwvrgsdvaary | | | |
| | | isvfgedsdrelaklhgvdvsedepdpiap | | | |
| | | lectrcgretprdeplcvwcgqamdpqaaa | | | |
| | | eldeaddreaealaelppekakrllevadv | | | |
| | | lddpeirstlldr | | | |
| WP_008312772 | integrase | mpvargtvymtdnpasavdtmvdrledghy | 412 | BJ1 | 25 |
| | [Haloarcula | disdadrdllldldrqirllgpsefsdhrh | | | |
| | amylolytica] | efllrrgliiakrvggladgvddreaaedi | | | |
| | | vqwinteqtgspetnkdyrvafrtigkivt | | | |
| | | dgdeypdavewvpggypdnydpapnpatml | | | |
| | | dwaddiqpmldaclnsrdralvalawdlgp | | | |
| | | rpgelydltpgdivdhdyglqvtlngkngr | | | |
| | | rspvlvpsvpyvrrwlddhpggdtdplwck | | | |
| | | lsspesisnnrvrdalkdvadragvdktvt | | | |
| | | pthfrkssasylasqgvsqahleehhgwtr | | | |
| | | gsdiasryiavfddasereiarahgldvea | | | |
| | | depdsvgpivcprceqktprekdacvwcgq | | | |
| | | vlsqsaaeeaerqrqdamdsmvaadsdlae | | | |
| | | aiatveaeigddvsirieglde | | | |
| WP_011023694 | integrase | msiheyytdiwlpkleekirtadypkrnrd | 390 | BJ1 | 26 |
| | [Methanosarcina | lilkfetylfseglkslrvlkylfvldkia | | | |
| | acetivorans] | sgssvsfskmnehhvqkiiadferselaas | | | |
| | | tkrdykviirrffkwlkgdkspaawikvsk | | | |
| | | kvsdqklpeymitedevkrmieaasnardk | | | |
| | | aiiallydsgcrigelggvkiknitfdqyg | | | |
| | | avvvvsgktgarrvrvtfaasylaawldvh | | | |
| | | pykekseafvfinlegvkkgeqmqyqafqy | | | |
| | | tlkkiakaagiekrihlhlfrhsrstelaq | | | |
| | | ylteaqmeehlgwaqgsemprtyvhlsgkq | | | |
| | | iddailgiygkkkkedtmpkltsrictrck | | | |
| | | kengptssfcaqcglpldpqavqevqvred | | | |
| | | amaqileqlmknkelrdlwnvaaegksses | | | |
| WP_049986559 | site-specific | msdsdqierlrervrnspticdadketllt | 423 | BJ1 | 27 |
| | integrase | fsdelefldveytdvrhikllqhcillagd | | | |
| | [Halobellus | sekytteelpdvaltstfgskdavkdlgrw | | | |
| | rufus] | irmydneetkrdyrialrmlgkrvtegddi | | | |
| | | peplqllsagtprsydptpdpakmlwwedh | | | |
| | | iepmiknahhlrdkaaiavawdsgarseef | | | |
| | | cglrvgdvsdhehgmkisvdgktgersfll | | | |
| | | ttatsyllqwlnvhpasndptaplwcklna | | | |
| | | pedtsyrmklkmlkkparragiehtditfr | | | |
| | | rmrkssasylasqnvnqahledhhgwkrgs | | | |
| | | niasryiavfgeandreiarahgvdvqtee | | | |
| | | heplapvtctrcrnetpmesfcvwcgqame | | | |
| | | hgaveeleaekreariellriaredptlld | | | |
| | | eidrleqvvgfvdsnpsilreardfvdasa | | | |
| | | d | | | |
| WP_052735531 | hypothetical | Mfkladaenflkseelsecnreilskyfry | 397 | BJ1 | 28 |
| | protein | lrhegnsertalnhmenmiwiakalhecdlg | | | |
| | [Methanosarcina | klaeddlylffdalenytytdragkvkkys | | | |
| | mazei] | eptketrkvslkkflkwnknyelhekikck | | | |
| | | rlkgkklpedikckedivkmieagsnsrdr | | | |
| | | aiiacfyesgarrgeqlsvklknveldeyg | | | |
| | | avitfipegktgarrvrlifsapylrewld | | | |
| | | dhprkddrdaplwctldknaghmsvtglvn | | | |
| | | vfnrcgekagiekkvnphsfrhdrathlaa | | | |
| | | nfteqqlkmylgwsptstqpatyvhlsgkn | | | |
| | | mddavlkmygikkaeddpeflkpgicprcr | | | |
| | | elttvnakfcykcglpltqeaattletikt | | | |
| | | eymqlsdldeiremknalkqeleeisklke | | | |
| | | mmlkagk | | | |
| WP_058994141 | site-specific | mtrnadrrienlqerieraeemsgddqnvl | 415 | BJ1 | 29 |
| | integrase | qafdnrlallgsqygkerrekllrhcvria | | | |
| | [Haloarcula | eevggladslddkraaedivrwihdtydne | | | |
| | sp. CBA1127] | esnrdyrvafrmfgkhvtdgdeipdsiswv | | | |
| | | sattskdynpmpnpakmlwweehilpmlde | | | |
| | | crhardkaliavawdsgarsgelrnltvgd | | | |
| | | vsdhkyglrisvdgkkgersitlvpsvphl | | | |
| | | rqwlnvhpgkdqpdaplwsklskpedisyq | | | |
| | | mklkilkkharkagidhtevtftqmrkssa | | | |
| | | sylasdgvnqahledhhgwdrgsdvasryv | | | |
| | | avfgdandraiaqahgvdveedesdpiapv | | | |
| | | tcprcrnetprdeptcvwcsqamdaaavee | | | |
| | | iereqkeirsellqiahddpdfldnldrve | | | |
| | | rfielgdenpeilrearafadates | | | |
| WP_066141378 | site-specific | mtadpagsierlmrversdtitpqdrenil | 415 | BJ1 | 30 |
| | integrase | afsnrmallrseysdqrhekllghitrmae | | | |
| | [Haladaptatus | qiedisdalddrkkaedvvrwinrnydnee | | | |
| | sp. R4] | tnkdyriafrvfakrvtdgddtpdsidwip | | | |
| | | sgysnnydpapnpknmlrwegdilpmvkgt | | | |
| | | rnsrdaalvtvawdsgarpgelqsltvgdv | | | |
| | | tdykhglqvtvegktgqrtvslipsvpylq | | | |
| | | rwltdhpdsgdpnaplwsklsspdqlsnrm | | | |
| | | lrkalnsaadragvkkpvnltnfrkssasy | | | |
| | | lasqnvnqahledhhgwtrgskvaaryvsv | | | |
| | | fggdsdreiarahgldvgedepdpiaplec | | | |
| | | prckretprqeefcvwcgqavepgaietme | | | |
| | | ndqretraallrlaqedpklldrveqlqdv | | | |
| | | maltdehpdllpdaqrfvntlred | | | |
| WP_076580843 | integrase | mpdirkqitslqdriersndisekdkqlll | 414 | BJ1 | 31 |
| | [Haloterrigena | afsdeidllkskysdhrhnkllrhctimae | | | |
| | daqingensis] | evgglsealedpgaakglvrwihmynneyt | | | |
| | | nhdyrtalrvfgqrvtegedyppgiewips | | | |
| | | gtssshdpvpdpadmlewetdilpmvdatm | | | |
| | | srdaalitvafdagpradelrtlsigdisd | | | |
| | | tehglriwvdgktgqrsvdlips | | | |
| | | vpylkrwlsdhpasddstaplwsklnspeg | | | |
| | | isyrqflnclkdaakragvtksvtptnlrk | | | |
| | | snatylarkgmnqafiedrqgrkrgsdata | | | |
| | | hyvarfgtdseaeyarlhgleveeeepepi | | | |
| | | gpvkcprcsketprhesscvwcnqvleyda | | | |
| | | idsiedaqrdirdvvlqfardd | | | |
| | | peiltdfqrnrelmdlfesnpdlyeeaqef | | | |
| | | veslpde | | | |
| WP_082224511 | site-specific | mtdqpktaikrnvercrerdglgdadaeai | 417 | BJ1 | 32 |
| | integrase | ldahhhmelvgnagvsdshhsdvlmravki | | | |
| | [Halolamina | aretepgtlaaaledrdaaedvvrwinrty | | | |
| | rubra] | dnpetnrgyrqafrafgrhslgvdelpecl | | | |
| | | dwvpagypsnydpapdpaqmlrwddhikpm | | | |
| | | legcnnvrdealvalcwdlgprtselhelq | | | |
| | | vgnisegdygltvtiengkngsrsptiwsv | | | |
| | | pfvrdwlerhpgdrddylwtrmdrpervsr | | | |
| | | nylrdalknaarrvdldlpatptptrfrks | | | |
| | | sasylasqnvnqafledhhgwvtgsdkaar | | | |
| | | yitvfsdqsdraiaeahgvdvdveddgpdm | | | |
| | | vecvrcealndadrsrcrqcdqvlsqeaae | | | |
| | | qealvdrvlsrlddqlleaddrderaelle | | | |
| | | gkqvveerrsdldvdalhqllssgda | | | |
| WPJ137035652 | recombinase Cre | mgnlsptnqtlpaiqaeedvlarlkefvqd | 349 | Cre | 33 |
| | [Rahnella | keafspntwrqlmsvmrichrwsiensrsf | | | |
| | sp. WP5]. | lpmlpadlrdylnwlqengrasstiathgs | | | |
| | | lismlhmaglippntsplvfravkkinrva | | | |
| | | vvtgertgqavpfrledlleldalwsdsis | | | |
| | | prhkrdlaflhvaystllriseiarlrvrd | | | |
| | | isratdgriilnvsytktivqtggliksln | | | |
| | | sqssrrltewlsvsginsepdaflfcpvhr | | | |
| | | sgsatlsvtrplstpaiesifaqawhtiga | | | |
| | | gepiipnkgryaawtghsarvgaaqdmagr | | | |
| | | gyavaqimqegtwkkpetlmryirnlqahe | | | |
| | | gamtdimekstqnhnntk | | | |
| WP_067435909 | recombinase Cre | mtdslpaplplhalsadadisarlaefvrd | 349 | Cre | 34 |
| | [Erwinia | kdafspntwrqllsvmricfswsqqngrsf | | | |
| | gerundensis] | lpmspddlrdylthlqeigrasstisthas | | | |
| | | lismlhrnaglvppntspavfrtmkkinrv | | | |
| | | aviagertgqavpfrlndlmaldrcwvnat | | | |
| | | rlqdlrnlaflhiaygtllrvselarlrvr | | | |
| | | dvtraedgriildvawtktivqtgglikal | | | |
| | | salstrrleawiaaaglarepdaflfcrvh | | | |
| | | rcnkallteeaplstpaieaifshawqtig | | | |
| | | paeparanksryrgwsghsarvgaaqdmak | | | |
| | | qgyavaqimqegtwkkpetlmryirnidah | | | |
| | | qgamvdlmerlrpdaesnn | | | |
| WP_081139620 | recombinase Cre | mnalvplspsdddlaqrlrefvqdkeafap | 337 | Cre | 35 |
| | [Pantoea latae] | ntwrqlmsvmrvchrwasannrtllpmspe | | | |
| | | dlrdylsylqsigrasstigthqslismlh | | | |
| | | rnaglvppstsplvsravkkinrvavvsge | | | |
| | | rtgqavpfrlsdlqkveaawaetpslrnmr | | | |
| | | dlaflhvaystlmrisevsrfrvgdvmrae | | | |
| | | dgriilegswtktildagslikalgskssa | | | |
| | | vvtkwivasglinepdaflfspvhrsgkvm | | | |
| | | vaidepmstpalksiftraweaagytdtak | | | |
| | | pnknryrrwsghsarvgaaqdlarkgysvp | | | |
| | | qimqegtwkkpetlmryiryveahkgamvd | | | |
| | | lmenqde | | | |
| WP_081365423 | hypothetical | mlqnekysgfpknrvnfiknltdytnvmvv | 391 | Cre | 36 |
| | protein | frnesllvpvhlrdmpmtnlpvnqtespll | | | |
| | [Citrobacter | itadkydervaenlhmffvdreaasentwa | | | |
| | freundii] | qmksvlrswglwckqfnkv | | | |
| | | wlpadpadvreyliylretlgrkkntiamh | | | |
| | | ksminkihreaglalpashilvtrgmkkis | | | |
| | | rqavlsgerveqaiplhlddlfqlaeitqa | | | |
| | | sgkmqqlrdlaflgvayntllrmsevarlr | | | |
| | | igdiqfqrdgsatldvgytktikdelgwkv | | | |
| | | lapdvagwlrnwlnasgltdestfifgkvd | | | |
| | | rygnahpavkpmagkniekifakaweavkg | | | |
| | | aplessryrtwtghsprvgaaqdmalkgte | | | |
| | | ltqimhegtwkrpeqvmsyiryidanksvm | | | |
| | | ldivnsqrmkr | | | |
| WP_084886047 | recombinase Cre | mnefsgftgvalsgaagddltakltafvrh | 342 | Cre | 37 |
| | [Pantoea septica] | reafspntwrqllsvmricwrwsqenhrsf | | | |
| | | lpmlpedmqdylfhlqatgrststisvhaa | | | |
| | | lmsmlhrnaglvpptvspdvvrakkkinrt | | | |
| | | avvsgerigqavpfcrpdlnrldklwkhsp | | | |
| | | rlqhlrdlafmhvaystllrmselsrlrvr | | | |
| | | ditraadgriildvgwtktilqsggivkal | | | |
| | | sarsserlmewisasgladepdailfcpvh | | | |
| | | rsnkittfttapmsapclediwrrarrqag | | | |
| | | daprvktnkgrysswsghsarvgaaqdmar | | | |
| | | kgisiaqimqegtwtqtqtvmryirmveah | | | |
| | | kgamiglmeeds | | | |
| YP_006472 | Cre [Escherichia | msnlltvhqnlpalpvdatsdevrknlmdm | 343 | Cre | 38 |
| | virus P1] | frdrqafsehtwkmllsvcrswaawcklnn | | | |
| | | rkwfpaepedvrdyllylqarglavktiqq | | | |
| | | hlgqlnmlhrrsglprpsdsnavslvmrri | | | |
| | | rkenvdagerakqalafertdfdqvrslme | | | |
| | | nsdrcqdirnlaflgiayntllriaeiari | | | |
| | | rvkdisrtdggrmlihigrtktlvstagve | | | |
| | | kalslgvtklverwisvsgvaddpnnylfc | | | |
| | | rvrkngvaapsatsqlstralegifeathr | | | |
| | | liygakddsgqrylawsghsarvgaardma | | | |
| | | ragvsipeimqaggwtnvnivmnyimldse | | | |
| | | tgamvrlledgd | | | |
| AAY91263 | site-specific | mgsitvrkrkdgsaaytaqirimqkgvtvy | 380 | DAI | 39 |
| | recombinase, phage | qesqtfdrkttaqawirkreaelhepgaie | | | |
| | integrase family | ranrsgvsvkemidqylkqyeklrplgktk | | | |
| | [Pseudomonas | ratlnaikeswlgdvtdaeltsqklveyav | | | |
| | protegens Pf-5] | wrmetfgiqaqtvgndlahlgavlsvarpa | | | |
| | | wgydvdphamsdarsvlrkmgavsrsrern | | | |
| | | rrptldeldriltyfeqmrdrrrqeidmlr | | | |
| | | vivfalfstrrqeeitrirwdllneseqsa | | | |
| | | lvtdmknpgqkygndvwchmpdeawrvlqs | | | |
| | | mpkvadevfpynsrsvsasftracnfleie | | | |
| | | dlhfhdlrhdgvsrlfemgwdipkvasvsg | | | |
| | | hrdwnsmrrythlrgngdpyagwqwiervi | | | |
| | | sgpvieaqvrvkrraagrap | | | |
| AEA60511 | integrase family | mgtivprkrkdgsigytaqirlkvkgkvvh | 358 | DAI | 40 |
| | protein | teaktfdrepaasawikkrerelsqpgaie | | | |
| | [Burkholderia | gakredptlgeviaryiredkrgigrtkkq | | | |
| | gladioli BSR3] | vletirgkdiaerpcselrsadyiqfarsl | | | |
| | | dvqpqtvgnymshlgaivriarpawgypla | | | |
| | | esefddamvvgkrlgltgksvardrrptpd | | | |
| | | elnrileyytemakreraelpmrelivfal | | | |
| | | fstrrqeeittirvedfegdrvlvrdmkhp | | | |
| | | gqkkgndtwcdvppeaarvieavrpksgpi | | | |
| | | fpynhrsisasftkacaflsiddlhfhdlr | | | |
| | | hegasrlfemglniphvaavtghrswsslk | | | |
| | | rythlrhvgdrwarwawldrvaplqeqs | | | |
| AGH34419 | shufflon-specific | mgsitarkgadgnvsyraairinkkgypay | 382 | DAI | 41 |
| | DNA recombinase | sesktfyskkvaenwlkkreveiqenpdil | | | |
| | [Acinetobacter | fgkeqlidltlsdaidkyldevgseygrtk | | | |
| | baumannii | ryalllikklpiarniitkihsthlaehva | | | |
| | D1279779] | lrrrgvpnlglepiatstqqhellhirgvl | | | |
| | | shasvmwgmdidlssfdkataqlrktrqis | | | |
| | | sskvrdrlptneelvtltkffaerwklnky | | | |
| | | gtkypmhlviwfaifscrreaeltrlwlqd | | | |
| | | ydsyhsswkvhdlknpngskgnhksfevle | | | |
| | | pcktivellldnevrsrmlqlgyderlllp | | | |
| | | lnpksigkefrdackmlgiedlrfhdlrhe | | | |
| | | gctrlaeqsftipeiqkvslhdswsslqry | | | |
| | | vsvksrrnviqleevlrlidet | | | |
| WP_003795408 | integrase | mgsivkrinpsgktvyraqiridraaypky | 387 | DAI | 42 |
| | [Kingella | aesrtfserrlaaawlkkreaeleanpell | | | |
| | oralis] | yyggkkqtiptlaqaieryfsepaatefgr | | | |
| | | tktatlkflsgypiaklpldkirradiaah | | | |
| | | inqrrdgwggflpvkpqtvnndlqyirsml | | | |
| | | khahfvwglnvnwaeidlaiegarrarlig | | | |
| | | kseermrlataqelqaltthfyqqwttrpn | | | |
| | | stkfpmhlimwfaiyscrreaeitrlawvd | | | |
| | | ydktagdwlvrdlkspsgskgnharflvnd | | | |
| | | klrqviaafrqpeiqnrlkwremqpet | | | |
| | | wliggdsksisasftrackllgiedlrfhd | | | |
| | | lrhegatrlaedgltvpqmqqitlhqswkt | | | |
| | | lqryvnlatrprenrldfadalavaqqkaa | | | |
| WP_024708115 | site-specific | mgtitarkkkksglivytaqiritrkgktv | 357 | DAI | 43 |
| | integrase | hsesqtfdrkklavawmnkregdllepggl | | | |
| | [Martelella | erakhgnvtladvidqyirenaapmgrtka | | | |
| | sp. AD-3] | qvlrtlkgydiadlpceeitsahiialare | | | |
| | | lsidkkpqtvanylshlssvfaiarpawgy | | | |
| | | pldrqamqdgvivakrlgmtsksrqrdrrp | | | |
| | | tleelgriltffrrrsiqapqsmpmdeivl | | | |
| | | falfstrrqdeicritwadldaqnsrvlvr | | | |
| | | dmknpgqkigndnwcdmpapamavirraaq | | | |
| | | kderifpyapesisanftracrligiedlh | | | |
| | | fhdlrhegisrlfeigyniphaaavsghrs | | | |
| | | wvslkryshirqrgdkyedwewmpdta | | | |
| WP_026380671 | site-specific | mgtitarkrkdgsvgyrarvrvmrdgmtyh | 356 | DAI | 44 |
| | integrase | etetfdrrpaaaawmkkrerelsrpgaipa | | | |
| | [Afifella | akfddptlakaidryieesvkeigrtkaqv | | | |
| | pfennigii] | lraikkhpivempcstikskdiieflqslt | | | |
| | | sqpqtvgnyashlaavfaiarpmwdyrlde | | | |
| | | remkdaitvarrlgiisrslqrdrrptlde | | | |
| | | ldkllahfierrkkapqalpmhkvivfalf | | | |
| | | strrqeeitriawkdfqkehkrvlvrdmkh | | | |
| | | pgeklgndtwvdlpseaiqiiesmrkskpe | | | |
| | | ifpystdaitanftracklldienlhfhdl | | | |
| | | rhegisrlfemgwniphvaavsghrswvsl | | | |
| | | krythiretgdkyagwgglrlavstk | | | |
| WP_033133807 | integrase | mgsvtarkgtdgsvsyraairinrkgypvy | 382 | DAI | 45 |
| | [Acinetobacter sp. | sesktfhskkmaenwlkkreveiqenpdil | | | |
| | MN12] | lgkekhidltladaidkyleevgseygrtk | | | |
| | | ryslllikkfpiarniitkiksvhladhva | | | |
| | | lrkagipllkldpiststqqhellhirgvl | | | |
| | | ahasvmwdididlnsfdkataqlrktrqis | | | |
| | | sskkrdrlptneelialtkyfverwklnkh | | | |
| | | gtkypmhlviwfaifscrreaeltrlsldd | | | |
| | | ydqyhsswkvhdlknpngskgnhksfdvld | | | |
| | | pckemikrlkqsevrermlrlghdenllip | | | |
| | | lnpkslgkefreackmlgiddlrfhdlrhe | | | |
| | | gctrlaeqsftipeiqkvslhdswsslqry | | | |
| | | vsvkarrsvmqledvlrlidet | | | |
| WP_064084314 | integrase | mgtitkrtnpsgavvyraqvrikkagapay | 383 | DAI | 46 |
| | [Eikenella | nesktftkkalaaewlkrreaeieanpdli | | | |
| | corrodens] | fgiqkmrmptlaaaidsylaelpavgrskk | | | |
| | | qgllflrgfriaalpldkitrdqvalfaqq | | | |
| | | rrnglpelglkpvkpptilqdiqyirvvik | | | |
| | | hafyvwnlnvswqeidfaieglergrivdr | | | |
| | | ptimrlpsseelqsltnhfyqayagrktta | | | |
| | | vpmhlimwlaiytcrrqdeicrmmladfdr | | | |
| | | ehgewlihdvkhpdgsrgndksfvispaai | | | |
| | | qvidellqdnvqrcmtrlggrpgslvplka | | | |
| | | ttisaqftrackvldirdlrfhdlrhegat | | | |
| | | rlaedgatipqiqrttlhdswsslqryvnl | | | |
| | | rrrgdrldfaeaianacapvkP | | | |
| WP_066317058 | site-specific | mativkrpkrdgsfsylaririartgqpdy | 351 | DAI | 47 |
| | integrase | sesktfpkkamaaewakrrelelaapggvl | | | |
| | [Halomonas | takwkgvtlndaierylhefadgagrskra | | | |
| | sp. G11] | tieqlrrfpiarvkitelsseqiidhaqmr | | | |
| | | rrsgvkpstaalditwlgiilktavaawrm | | | |
| | | pvdlnefesaklllrskglinrpasrdrrp | | | |
| | | tpeeieqirayfqhsqkirpsaiipmedim | | | |
| | | dfaiassrrqeeitrltwddldteamtcwv | | | |
| | | rdakhprqkwgnhkrfkltheamaiiqrqp | | | |
| | | rkrdeprifpyysrsigtrwraateskgie | | | |
| | | dlrfhdlrheatsrlfeagyeivevqqftl | | | |
| | | heswdvlkrythlrpeklqlr | | | |
| WPJ182277758 | integrase | matitkrrnpsgetvyrvqvrvgkkgypaf | 384 | DAI | 48 |
| | [Neisseria | nesrtfskkalavewgkkreaeieagpell | | | |
| | gonorrhoeae] | fkrgkvkmmtlseamrkylnetlgagrskk | | | |
| | | mglrflmefpiggigidklkrsdfaehvmq | | | |
| | | rrrgipeldiapiaastalqelqyirsvlk | | | |
| | | hafyvwgleigwqeldfaanglkrsnmvak | | | |
| | | sairdrlptteelqtlttyflrqwqsrkss | | | |
| | | ipmhlimwlaiytsrrqdeicrllfddwhk | | | |
| | | ndctrsvrdlknpngstgnnkefdilpmal | | | |
| | | pvidelpeesvrkrmlankgiadslvpcng | | | |
| | | ksvsaawtrackvlgikdlrfhdlrheaat | | | |
| | | rmaedgftipqmqrvtlhdgwnslqryvsv | | | |
| | | rkrstrldfkeammqaqsdiksgk | | | |
| WP_087542849 | integrase | mgtisqrkladgtirfraeirisrkglanf | 380 | DAI | 49 |
| | [Acinetobacter sp. | kesktfssmrlaqkwlamreeeieenpeil | | | |
| | WCHA29] | lgrsdvtnitlanaiekyldevgneygrtk | | | |
| | | tyclrliqkfpiaqhiitkikpadisdhva | | | |
| | | lrkngydkldlkpiatstlqhellhirgvl | | | |
| | | shasvmwdvnvdlagfdkataqlrktrqis | | | |
| | | ssgkrdrlpttvelkklteyfyrkwqnpvy | | | |
| | | sypmhlimwfaifscrreaeitemlladhd | | | |
| | | vdnevwkvrdlknpkgskgnhkefnvlepc | | | |
| | | qkmiellqrkdvrkrmlkrgydkdllipls | | | |
| | | prtiggefrnackllgiedlrfhdlrhegc | | | |
| | | trlaeqgftipqiqqvslhdswgsleryvs | | | |
| | | vkkrkktielaevlpliged | | | |
| AAB59340 | recombinase (FLP) | mpqfgilcktppkvlvrqfverferpsgek | 423 | Flp | 50 |
| | (plasmid) | ialcaaeltylcwmithngtaikratfmsy | | | |
| | [Saccharomyces | ntiisnslsfdivnkslqfkyktqkatile | | | |
| | cerevisiae] | aslkklipaweftiipyygqkhqsditdiv | | | |
| | | sslqlqfesseeadkgnshskkmlkallse | | | |
| | | gesiweitekilnsfeytsrftktktlyqf | | | |
| | | lflatfincgrfsdiknvdpksfklvqnky | | | |
| | | lgviiqclvtetktsvsrhiyffsargrid | | | |
| | | plvyldeflrnsepvlkrvnrtgnsssnkq | | | |
| | | eyqllkdnlvrsynkalkknapysifaikn | | | |
| | | gpkshigrhlmtsflsmkglteltnvvgnw | | | |
| | | sdkrasavarttythqitaipdhyfalvsr | | | |
| | | yyaydpiskemialkdetnpieewqhieql | | | |
| | | kgsaegsirypawngiisqevldylssyin | | | |
| | | rri | | | |
| NP_040495 | hypothetical protein | matfsklserkrstfikysreirqsvqydr | 372 | Flp | 51 |
| | (plasmid) | eaqivkfnyhlkrphelkdvldktfapivf | | | |
| | [Lachancea | evsstkkvesmvelaakmdkvegkgghnav | | | |
| | fermentati] | aeeitkivraddiwtllsgvevtiqkrafk | | | |
| | | rslraelkyvlitsffncsrhsdlknadpt | | | |
| | | kfelvknrylnrvlrvlvcetktrkpryiy | | | |
| | | ffpvnkktdplialhdlfseaepvpksras | | | |
| | | hqktdqewqmlrdslltnydrfiathakqa | | | |
| | | vfgikhgpkshlgrhlmssylshtnhgqwv | | | |
| | | spfgnwsagkdtvesnvarakyvhiqadip | | | |
| | | delfaflsqyyiqtpsgdfelidsseqptt | | | |
| | | finnlstqedisksygtwtqvvgqdvleyv | | | |
| | | hsyamgklgirk | | | |
| NP_040496 | hypothetical protein | msefselvrilpldqvaeikrilsrgdpip | 474 | Flp | 52 |
| | (plasmid) | lqrlaslltmviltvnmskkrksspiklst | | | |
| | [Zygosaccharomyces | ftkyrrnvakslyydmssktvffeyhlknt | | | |
| | bailii] | qdlqegleqaiapynfvvkvhkkpidwqkq | | | |
| | | lssvherkaghrsilsnnvgaeisklaetk | | | |
| | | dstwsfiertmdlieartrqpttrvayrfl | | | |
| | | lqltfmnccrandlknadpstfqiiadphl | | | |
| | | grilrafvpetktsierfiyffpckgrcdp | | | |
| | | llaldsyllwvgpvpktqttdeetqydyql | | | |
| | | lqdtllisydrfiakeskenifkipngpka | | | |
| | | hlgrhlmasylgnnslkseatlygnwsver | | | |
| | | qegvskmadsrymhtvkksppsylfaflsg | | | |
| | | yykksnqgeyvlaetlynpldydktlpitt | | | |
| | | neklicrrygknakvipkdallylytyaqq | | | |
| | | krkqladpneqnrlfssespahpfltpqst | | | |
| | | gsstpltwtapktlstglmtpgee | | | |
| XP_004178636 | hypothetical protein | mpreknsivasgkvdaysnsnvrelirafk | 514 | Flp | 53 |
| | TBLA_0B02750 | ecktvqdyfiiliqvrfeiyeelfqelfgk | | | |
| | [Tetrapisispora | dkviidkrifgsllsyyilhtfpkikrvty | | | |
| | blattae CBS 6284] | gtyrknkaitinsleidysrhkiqfkyris | | | |
| | | gnrliqlqtflneqsffkpwkfrilsdgrk | | | |
| | | eenlfiidknplknhnepntnskhirnset | | | |
| | | nlkfnqnvleylnkngdpwdiysqcfamfe | | | |
| | | nhsremsciryklisvltftnacrisdlir | | | |
| | | ldpssfhlkknkylgtivcghtfntlnnip | | | |
| | | rtvqfipaytrgcdmlqlleeylkinkngp | | | |
| | | feyvpmqnnkspiqttndvnqkyqffkegv | | | |
| | | gaaytklmsvhpahhlfklknapktdlgiy | | | |
| | | lminylnkiglqneghrlgnwtkvcpidgs | | | |
| | | elkkrnftttltpchsvrdstraiisgyyq | | | |
| | | iskytnnnkkrmvrvhtlpeeptsftysdn | | | |
| | | lqlhyghwakivphdvlaflleysvtskea | | | |
| | | rlaldtlpeiltpslsmpytsssssssdds | | | |
| | | hsyh | | | |
| XP_018218754 | hypothetical protein | mskfdilyktppkvlvsqfiarfgepsgek | 423 | Flp | 54 |
| | DI49_5675 (plasmid) | lascaaeltylcwmithngaaikratflsy | | | |
| | [Saccharomyces | ntiiskslqydvvkktlqfkyktqkaailq | | | |
| | eubayanus] | aslqklipgweftiipyygqkeqsdvtdiv | | | |
| | | snlqlqfespeevekgnshskkmlkallne | | | |
| | | desvwniaekildsfeytsrytktkaqyqf | | | |
| | | lflatfvncarfsdiknvdpqsfkliqney | | | |
| | | lgviiqclvtetktgvsrhiyffsakgrld | | | |
| | | slvyldeflrysepvpkrinktssssgnkq | | | |
| | | qyqllkdnlvrsynkalksnapysilaikn | | | |
| | | gpkshigrhlmtsflsmkglteltnvvgnw | | | |
| | | sdkrasvvarttythqvtaipdhyfalvsg | | | |
| | | yygydqiskemipwkdetnpieewrhieql | | | |
| | | kgstggstryaawngiiaqevldylssyis | | | |
| | | rri | | | |
| CAF28569 | putative phage | meiemnkanydeilqdyffskslrpatews | 326 | IntC | 55 |
| | integrase [Yersinia | yrkvinsfrryigdnllpgevdrltvlnwr | | | |
| | pseudotuberculosis] | rhvlnkqglssitwnnkvahmraifnhall | | | |
| | | hdlvsfknnpfngvivrpdvkrkktltqse | | | |
| | | ikkiylimearereehvgimgksrsalrpa | | | |
| | | wfwltvvdtlrytgmrqnqllhirlgdvnl | | | |
| | | ndgwinlrpeasknhkehripiarvlrprl | | | |
| | | erlvataiekganqvdqlfnisridgrket | | | |
| | | vtenmdspplrsffrrlsvecrctisphrf | | | |
| | | rhtiatemmkspdrnlkvvqtllghssiav | | | |
| | | tleyvegdidslrlaleetferkevf | | | |
| CAF29071 | Putative site- | mqhncnlkypdevskllilqwrkavvgksi | 270 | IntC | 56 |
| | specific | ievtwnsyvrqlktifkfgienqflpftkn | | | |
| | recombinase | pfdglfiregkrkrkvyspsdldrlsfgik | | | |
| | (plasmid) | eskylpailrplwftralimtfrytairrs | | | |
| | [Haemophilus | qlnklrirdidllnqvihispeinknheyh | | | |
| | influenzae] | ilpishtlypyldnllnelkkmkqsadaql | | | |
| | | fninlfskavkrrgkemtadqisylfkvis | | | |
| | | khtgvnssphrfrhtaatnlmknpenlyvv | | | |
| | | kqllghkdikvtlsyiesdisslrkhidel | | | |
| CAX67909 | probable phage | metnitwqqlideyffakplrsasewsytk | 337 | IntC | 57 |
| | integrase | vfksfvhymgplscpndvtyhkvlawrrfl | | | |
| | [Salmonella bongori] | lkekklsgrtwnnkvahmraifnygiqrgl | | | |
| | | lqydenpfnnsvvkpdkkrkktltqaqiey | | | |
| | | ayqimeqyenqentglglkysrcalfpawf | | | |
| | | wltvldtlyytgirqnqllhirlndvdlre | | | |
| | | gqirlitegcknhkehyvpvisflrprltc | | | |
| | | lvekaqseglkgndrlfnialftgkdpaig | | | |
| | | ddmdspqvraffrrlskecqfaisphrfrh | | | |
| | | tlatemmkmpeqnlhmaqsvlghsnmkstl | | | |
| | | eyvendiavmgraleaqfmqikaaharsiy | | | |
| | | sgltknr | | | |
| WP_011817054 | site-specific | mememnqvnyddilqdyffskslrpatews | 327 | IntC | 58 |
| | integrase [Yersinia | yrkvinsfirryigdnllpgevdrqivlnw | | | |
| | enterocolitica] | rrhvlnkqglssitwnnkvahmraifnhal | | | |
| | | lydlvvlkhnpfngvivrpdvkrkktltqs | | | |
| | | eiekiylimearereehvgimdksrsalrp | | | |
| | | awfwltvvdilrytgmrqnqllhirlgdvn | | | |
| | | lndgwinlrseasknhkehrvpiarvlrpr | | | |
| | | lerlvaaaidkganqadqlfnisrfdgrke | | | |
| | | sitenmdnpplrsffrrlsvecrctisphr | | | |
| | | frhtiatemmkspdrnlkvvqtllghssia | | | |
| | | vtleyvegdidslrlaleetferkavff | | | |
| WPJI24108415 | tyrosine recombinase | mtdigyesllddyffskslrpatewsyrkv | 318 | IntC | 59 |
| | XerD [Dickeya | tnsfirfasdippcrvdraavlhwrrhllt | | | |
| | dianthicola] | ekkvsartwnnkvahmraifnhgiktrllp | | | |
| | | htenpfnnvitrpdmkrkktlaagqldaid | | | |
| | | rlmeqhlelerqgmgvnfnecalypawfwk | | | |
| | | tvldtlrytgmrqnqllhirlsdvnldlgi | | | |
| | | inlrpegsknhrehrvpvisvlrqglsrli | | | |
| | | eesvareaqpdeqlfnvyrfigrasndrnv | | | |
| | | prnseiplrsffrrlsnecrftvsphrfrh | | | |
| | | tlatemmkspdrnlqivknllghssltttl | | | |
| | | eyvesnidsiraalegelrc | | | |
| WP_034939985 | site-specific | meqrmtfediltdyffskvlrpatewsyrk | 319 | IntC | 60 |
| | integrase [Erwinia | vvktftefcgddinpehitrmdilkwrrhv | | | |
| | mallotivora] | lveqklskrtwnnkvshmraifnhaishkl | | | |
| | | tshednpfsmvvvrpdikrkktltdeqikk | | | |
| | | aclvmerkimeeergthehranalkpawfw | | | |
| | | mtvidtlrytgmrqnqllhirlcdvdlkng | | | |
| | | vinlcpegsknhrehrvpvtdrlrpglavl | | | |
| | | harsvdkgakpedqlfninrftykknvqgk | | | |
| | | nmdhpplrsffrrlsrecgciisphrfrht | | | |
| | | iatdlmkrperslndvqmllghsslavtle | | | |
| | | yveanidnlrknleaafaf | | | |
| WP_071921402 | recombinase XerD | mensitfgeiienyffsktlrnatewsyrk | 319 | IntC | 61 |
| | [Kosakonia | vlksflhfaggnmmpedvddklvinwrrhv | | | |
| | radicincitans] | ineeglskitwnnklthmralfnysmaegy | | | |
| | | vshkknpfngkiarpdvkrkktltdiqikk | | | |
| | | tyllmesreideftgnietrrnalkpawfw | | | |
| | | ftvldtfsrtgmrqnqllhirlrdvdlehs | | | |
| | | wislcpegsknhkehrvpitamlrprlesl | | | |
| | | ynkavergaglndqlfnvsrfdvnrketat | | | |
| | | nmdnpplraffrrlskecgfvvsphrfrht | | | |
| | | iatnlmrlpdmikltqdllghstpavtlqy | | | |
| | | vesdidkvrsvleqldaa | | | |
| WP_080281299 | site-specific | mkseekmhdeweflleeyfftkqlrsatew | 343 | IntC | 62 |
| | integrase [Serratia | syrkvvltftrfiggtitpamvtqrdvllw | | | |
| | marcescens] | rrhllkeknlsvhtwnnkvahlraifnlgi | | | |
| | | kktliqhtenpfngtvvrsdtkkkriltks | | | |
| | | qltrlylvmqqyeqrekerkpvkggrcaly | | | |
| | | ptwfwmtvldtfrytgmrnnqmihirlrdv | | | |
| | | nleqgwielrlegskthrewkvpvvrqlre | | | |
| | | rikllimratergagqhdllfdvkrftspr | | | |
| | | hahyiydeknvlqsfrsfyrrlsresgfdi | | | |
| | | sshrfrhtlatelmkspdmlklvkdllghr | | | |
| | | nvsttmeyieldmevagkaleqelvlhtdi | | | |
| | | tatrslqsltqa | | | |
| WP_080859203 | recombinase XerD | mkekitwtefveeyilekelrtasewsyrk | 333 | IntC | 63 |
| | [Citrobacter braakii] | vsscfaehlgpfvfpedvtrrhallwrrr | | | |
| | | vlkvekrqettwnnkashmnalfnya | | | |
| | | ikrrlfeidenpfaetkvkagkkkkktmrq | | | |
| | | aqishayrvmeaheeeerrlgilasmalfp | | | |
| | | awfwltmmdtlyytgmrqnqllhlrvgdif | | | |
| | | ldeniirlgnkgsknhqehflsvvsylkpr | | | |
| | | lalilqkaaerglkkndllfnipvftgkde | | | |
| | | nitedmgsppvrsffrrlsrecgftmtshr | | | |
| | | frhtlatemmklpeqnlyitrnvlghssmk | | | |
| | | stleyverdldaerrvlekqfavlkkhkvi | | | |
| | | dhcdedg | | | |
| ABQ80725 | phage integrase | mcaqtarlsdrqlkavkpkdkdyvltdgdg | 418 | IntG | 64 |
| | family protein | lqlrvrvnrsmqwnfnyrhpvtknrinmal | | | |
| | [Pseudomonas putida | gsypevslaqarrkavearevlaqgidpka | | | |
| | FI] | qrndlaqaklaetehtfekvasawfelkkd | | | |
| | | svtpayaediwrsltlhvfpsmkstpisev | | | |
| | | sapmvikilrpieskgsletvkrlsqrlne | | | |
| | | imtygvnsgmifanplsgiravfkkpkken | | | |
| | | maalppeelpelmleianasikrttrclie | | | |
| | | wqlhtmtrpaeaattrwvdidferrvwtip | | | |
| | | permkksrphsiplsdqamslleilkshsg | | | |
| | | hreyvfpadrnprthansqtanmalkrmgf | | | |
| | | qdrlvshgmrsmastilnehgwdpelieva | | | |
| | | lahvdkdevrsaynradyierrrpmmawws | | | |
| | | eyilkastgnlsasamnvardrnvvpir | | | |
| EAQ07179 | symbiosis island | mplsdiqvmlkprekaykvsdfeglfvlvk | 395 | IntG | 65 |
| | integrase [Yoonia | pngsklwqfkyrmdgkerllsigvypnisl | | | |
| | vestfoldensis | aqarktkdgaranvaagidpseakqqekrq | | | |
| | SKA53] | rrevndqtfeklgaeffakqrkegksaads | | | |
| | | kteyhlqlasrdfgrkpiieitapmilktl | | | |
| | | rkveakghyetahrlrsrigsiffyavasg | | | |
| | | iaetdptyalrdalirptrkhraaiidpqa | | | |
| | | lgrlmneidvfegqattrialkllamvaqr | | | |
| | | pgeirhakwseidfvkkvwsipadrmkmrr | | | |
| | | dhivplpdqaialldqlrrmngngeylfps | | | |
| | | lrtwkrpmsentlnaalrrmgysgdemtah | | | |
| | | gfrasfstlanesglwnpdaieralahvek | | | |
| | | nevrrayargehweervrlanwwagylenl | | | |
| | | qam | | | |
| EAY64047 | Phage integrase | mavrgfllqtstsdhqwkqppiwgsfggfa | 447 | IntG | 66 |
| | [Burkholderia | khplqtpprhqhmaltdlkvrtakpaekqq | | | |
| | cenocepacia PC184] | klydgsgllllitpaggkrwifkyridgke | | | |
| | | kslalgtypdislaearsrrdsareklaag | | | |
| | | ldpseakkadkraaqlaaassfeivarewf | | | |
| | | etqrggwsevyagkvinclevdvfprlgar | | | |
| | | piasidapellaiirtvesrgvretakrvl | | | |
| | | qrsravfqygimtgrcampaadidaetvlk | | | |
| | | kstgvqhmarvkvteipqlmrdideysgdl | | | |
| | | vtrlalrfmaltfvrtkemiqaewpeidvg | | | |
| | | aaewrvpaermkmrdphivplsrqaldvla | | | |
| | | qlreingqqrfvfysvqgrshisnntmlya | | | |
| | | lyrmgyksrmtghgfrglaattlrelgysr | | | |
| | | dvverqmahaernqvtaayvhaeylperrk | | | |
| | | mmqhwadhldelragakiipitastp | | | |
| WP_009758561 | DUF4102 domain- | maltdarirnlkprekpfktadydglyvlt | 395 | IntG | 67 |
| | containing protein | npngsklwrlkyrfmdkerlltlgkypsvs | | | |
| | [Ahrensia sp. | ladarqarddarerlaqgqdpndtkrqktl | | | |
| | R2A130] | aakishgnsfskiaeqymakiikegraest | | | |
| | | lakidwlmdmanadlgskpiteitspmvlh | | | |
| | | tlkkvetkgnyetakrlrsqigavfrfaia | | | |
| | | nalaendptfalrdalvnvkatpraaildk | | | |
| | | avlgglmrsidgfdgqtttrlgmellaivv | | | |
| | | trpgelrharweefdfdqavwavpaprmkm | | | |
| | | rkphfvplparaleileelrmlngwgqlvl | | | |
| | | psikssirpmsentmnaalrrmgyggdemt | | | |
| | | shgfratfstianesglwnpdaiekalahv | | | |
| | | eankvrgayargqywdervrmanwwsglls | | | |
| | | dlrtq | | | |
| WP_034388214 | DUF4102 domain- | maltdakiralkpkgksykvsdfgglylsv | 398 | IntG | 68 |
| | containing protein | tskgsklwrqkyrfngkegtlsfgpypevs | | | |
| | [Hellea balneolensis] | lkeardqrdeakanlkkglnpadlkrkaaa | | | |
| | | eelgkseytfnkvadnfvkkltkegrspat | | | |
| | | lskldwllkdarkdfghmpiatitapiilk | | | |
| | | tlrkretqehyetasrmrsriggvfryava | | | |
| | | sgitdtdptyalrdalirptvthraaivtk | | | |
| | | dglaelvmaideyrgsrqtaialkllmqfa | | | |
| | | crpgeirqakweefnfeecvwsipsnrmkm | | | |
| | | rrphkvpltksslllleelkeltgwgeflf | | | |
| | | jpaqtsskkpmsdntmnqalvrmgfrkdev | | | |
| | | tphgfrstfstfanesglwapdvieaycar | | | |
| | | qdrnavrraynrslywgervklanwwanil | | | |
| | | cnitthhdd | | | |
| WP_059187617 | DUF4102 domain- | malsdvkcrnarpasklfklsdggglqlwv | 407 | IntG | 69 |
| | containing protein | qptgsrlwrlayrfdgkqkllalgsyplis | | | |
| | [Mesorhizobium loti] | laearqarddakrlllagmdpaherrsrka | | | |
| | | gsakdtfrsiaeeyvdklkkegradrtitk | | | |
| | | vkwlldfayptigdtcireidaatilvalr | | | |
| | | svevrgryesarrlrstigsvfryaiatar | | | |
| | | agtdptsalrdalirpivtpraaitepkal | | | |
| | | ggllraidafdgqttsrtalklmallfprp | | | |
| | | gelrgaeweefdfessvwtipetrmkmrrp | | | |
| | | hrvplsrqaitilirlreisgagtllfpsv | | | |
| | | rstsrpisdntlnaalrrmgyskeeatahg | | | |
| | | fratastllnecgkwhpdaierqlahiekn | | | |
| | | dvrrayaraehweervrmvqwwadyldkig | | | |
| | | nakterrplapkalrye | | | |
| WP_065323774 | DUF4102 domain- | mpvlsdakvralkpkekpykqadfdglfll | 403 | IntG | 70 |
| | containing protein | vnpggsklwrfkyrwmgkekllsfgkypdl | | | |
| | [Epibacterium | slkqardqrddarkllaegkdp | | | |
| | mobile] | sferkraqtakeaehretfsrladallekk | | | |
| | | rlegksastlaktewlhgllcadlgaypis | | | |
| | | qisardvlvplrkmeakgrnesalrmrsaa | | | |
| | | gqifryaiaqgliendptfglrdaltrapv | | | |
| | | rhrsalidpekvgglmraiagfdgqpttrl | | | |
| | | alqllavtalrpgelrmaewseidldkaiw | | | |
| | | tvpahrakmrrphmvplspealgklrelqe | | | |
| | | ltgwgqllfpsirsskrcmsentlnaalrr | | | |
| | | mgysgedmtahgfratfstlanesglwsad | | | |
| | | aieralahvegneirkayargthwdervri | | | |
| | | aawwagylqqladnagqhqtp | | | |
| WP_069879560 | DUF4102 domain- | mpltdtaiknakalskvrklsdggglqlwl | 407 | IntG | 71 |
| | containing protein | mptgaklwrlayrfdgkqrklsigaypgid | | | |
| | [Bosea sp. | lkaaraareeakehlragrdpseqkrldri | | | |
| | BIWAKO-01] | tkqetrattftslaaelkakkqregkaegt | | | |
| | | iekfewllsmaekdlgkrpvaeisaaevls | | | |
| | | vlrksekrghletakrlrsvigqvfryaia | | | |
| | | agkvandptlalrgalampkptsraaitdp | | | |
| | | krlgallraidgyegqnqtraalqlmallf | | | |
| | | qrpgelrsaewsefnldeavwlipaarmkm | | | |
| | | rrehavplprqalltleelreisdrspllf | | | |
| | | pslrsasrpmsdvtmnaalrrlgyakdemt | | | |
| | | phgfratastllnecgkwssdaiekalahq | | | |
| | | ernavrrayargehwqervrmaqwwadyld | | | |
| | | tlrngatiipmpakdtg | | | |
| WP_076486125 | DUF4102 domain- | mplsdvtirnlkprdrsykvsdfdglfvlv | 396 | IntG | 72 |
| | containing protein | kptgarlwqfkyridgkekllsigrypeig | | | |
| | [Rhodobacter | laqarlardearsmvangrdpsaakqerkr | | | |
| | aestuarii] | aelerrgvtfetqaqaflektrkeglastt | | | |
| | | laknewllamaiadfgakpmseisaqmilr | | | |
| | | clrkveakgnyetakrlrakisavfryava | | | |
| | | ngvaetdptyalrdalvrpkakpraaiidp | | | |
| | | qalgglmraietytgqrvtkialellalmv | | | |
| | | prpgelrqarweeidldariwaipaermkm | | | |
| | | rrphriplsdravrllhelreltgwtgfll | | | |
| | | pslvsprrvmsentlntalrrmgfgademt | | | |
| | | shgfrasfstlanesglwnpdaieralahi | | | |
| | | eqndvrrayargehwdervrlaqwwadyle | | | |
| | | tlrtsa | | | |
| WP_084396548 | DUF4102 domain- | mpltdiqlrqlkprekdyktadggglyvhv | 399 | IntG | 73 |
| | containing protein | sktgsrlwrfryrfdgkqkllafgaypais | | | |
| | [Henriciella | lararelraeaktllaegidpaahakaeka | | | |
| | aquimarina] | qqaaltehtfekiaaelveklrkegkadvt | | | |
| | | ltkkqwlldmanadfgdrpitaitaadilt | | | |
| | | tlrkveakgnyetakrlrstigqvfryaia | | | |
| | | taraendptyglrgalvapkvshmaaitdw | | | |
| | | dgfgdliraiwdyeggspstraalklmall | | | |
| | | ytrpgelrlalwdefdlekstwtipaartk | | | |
| | | mrrehtkplpslavdilktlraetgsnyrv | | | |
| | | fpssiardkpisentlnqalrrmgfdkheh | | | |
| | | tshgfratassllnesglwnadaieaelgh | | | |
| | | vgadevrrayhrarywdervrmadwwanqi | | | |
| | | tktistarl | | | |
| AAO32355 | IntI3 integrase | mnrynrndkpdwvpprsiklldqvrervry | 346 | IntI | 74 |
| | (plasmid) | lhyilqtekayvywakafvlwtarshggfr | | | |
| | [Klebsiella | hpremgqaevegfltmlatekqvapathrq | | | |
| | pneumoniae] | alnallflyrqvlgmelpwmqqigrpperk | | | |
| | | ripvvltvqevqtllshmagteallaally | | | |
| | | gsglrlrealglrvkdvdfdrhaiivrsgk | | | |
| | | gdkdrvvmlpralvprlraqliqvravwgq | | | |
| | | dratgrggvylphalerkyprageswawfw | | | |
| | | vfpsaklsvdpqtgverrhhlfeerlnrql | | | |
| | | kkavvqagiakhvsvhtlrhsfathllqag | | | |
| | | tdirtvqellghsdvsttmiythvlkvaag | | | |
| | | gtsspldalalhlspg | | | |
| AAT72891 | IntI2 [Shigella | msnspflnsirtdmrqkgyalktektylhw | 325 | IntI | 75 |
| | sonnei] | ikrfilfhkkrhpqtmgseevrlflsslan | | | |
| | | srhvaintqkialnalaflynrflqqplgd | | | |
| | | idyipaskprrlpsvisanevqrilqvmdt | | | |
| | | rnqviftllygaglrineclrlrvkdfdfd | | | |
| | | ngcitvhdgkggksrnsllptrlipaikxl | | | |
| | | ieqarliqqddnlqgvgpslpfaldhkyps | | | |
| | | ayrqaawmfvfpsstlcnhpyngklcrhhl | | | |
| | | hdsvarkalkaavqkagivskrvtchtfrh | | | |
| | | sfathllqagrdirtvqellghndvkttqi | | | |
| | | ythvlgqhfagttspadglmllinq | | | |
| ACJ39716 | IntI1 [Acinetobacter | mktataplpplrsvkvldqlrerirylhys | 344 | IntI | 76 |
| | baumannii AB0057] | lrteqayvnwvrafirfhgvrhpatlgsse | | | |
| | | veaflswlanerkvsvsthrqalaallffy | | | |
| | | gkvlctdlpwlqeigrprpsrrlpvvltpd | | | |
| | | evvrilgflegehrlfaqllygtgmriseg | | | |
| | | lqlrvkdldfdhgtiivregkgskdralml | | | |
| | | peslapslreqlsrarawwlkdqaegrsgv | | | |
| | | alpdalerkypraghswpwfwvfaqhthst | | | |
| | | dprsgvvrrhhmydqtfqrafkraveqagi | | | |
| | | tkpatphtlrhsfatallrsgydirtvqdl | | | |
| | | lghsdvsttmiythvlkvggaasngrlrkv | | | |
| | | lpasadgrqqpvva | | | |
| WP_069970415 | class 1 integron | mktataplpplrsvkvldqlrerirylhys | 337 | IntI | 77 |
| | integrase IntI1 | lpteqayvhwvrafirfhgvrhpatlgsse | | | |
| | [Klebsiella | veaflswlanerkvsvsthrqalaallffy | | | |
| | pneumoniae] | gkvlctdlpwlqeigrprpsrrlpvvltpd | | | |
| | | evvrilgflegehrlfaqllygtgmriseg | | | |
| | | lqlrvkdldfdhgtiivregkgskdralml | | | |
| | | peslapslreqlsrarawwlkdqaegrsgv | | | |
| | | alpdalerkypraghswpwfwvfaqhthst | | | |
| | | dprsgvvrrhhmydqtfqrafkraveqagi | | | |
| | | tkpatphtlrhsfatallrsgydirtvqdl | | | |
| | | lghsdvsttmiythvlkvggagvrxpldal | | | |
| | | ppltser | | | |
| WP_071681306 | class 1 integron | mktataplpplrsvkvldqlrerirylhys | 337 | IntI | 78 |
| | integrase IntI1 | lpteqayvhwvrafirfhgvrhpatlgsse | | | |
| | [Citrobacter | veaflswlanerkvsvsthrqalaallffy | | | |
| | freundii] | gkvlctdlpwlqeigrprpsrrlpvvltpd | | | |
| | | evvrilgflegehrlfaqllygtgmriseg | | | |
| | | lqlrvkdldfdhgtiivregkgskdralml | | | |
| | | peslapslreqlsrarawwlkdqaegrsgv | | | |
| | | alpdalerkypraghswpwfwvfaqhthst | | | |
| | | dprsgvvrrhhmydqtfqrafkraveqagi | | | |
| | | tkpatphtlhhsfatallrsgydirtvqdl | | | |
| | | lghsdvsttmiythvlkvggagvrspldal | | | |
| | | ppltser | | | |
| NP_037686 | integrase | mgrrrsherrdlppnlyirnngyycyrdpr | 357 | Lambda | 79 |
| | [Escherichia virus | tgkefglgrdrriaiteaiqaniellsgnr | | | |
| | HK022] | reslidrikgadaitlhawldryetilser | | | |
| | | girpktlldyaskirairrklpdkpladis | | | |
| | | tkevaamlntyvaegksasaklirstlvdv | | | |
| | | freaiaeghvatnpvtatrtaksevrrsrl | | | |
| | | taneyvaiyhaaeplpiwlrlamdlavvtg | | | |
| | | qrvgdlcrmkwsdindnhlhieqsktgakl | | | |
| | | aipltltidalnisladtlqqcreassset | | | |
| | | iiaskhhdplspktvskyftkarnasglsf | | | |
| | | dgnpptfhelrslsarlymqigdkfaqrll | | | |
| | | ghksdsmaaryrdsrgrewdkieidk | | | |
| NP_037720 | integrase | mgrrrsherrdlppnlyirnngyycyrdpr | 356 | Lambda | 80 |
| | [Escherichia | tgkefglgrdrriaiteaiqanielfsghk | | | |
| | virus HK97] | hkpltarinsdnsvtlhswldryekilasr | | | |
| | | gikqktlinymskikairrglpdapledit | | | |
| | | tkeiaamlngyidegkaasaklirstlsda | | | |
| | | freaiaeghittnpvaatraaksevrrsrl | | | |
| | | tadeylkiyqaaesspcwlrlamelavvtg | | | |
| | | qrvgdlcemkwsdivdgylyveqsktgvki | | | |
| | | aiptalhvdalgismketldkckeilgget | | | |
| | | iiastrreplssgtvsryfmrarkasglsf | | | |
| | | egdpptfhelrslsarlyekqisdkfaqhl | | | |
| | | lghksdtmasqyrddrgrewdkieik | | | |
| NP_040609 | integration protein | mgrrrsherrdlppnlyirnngyycyrdpr | 356 | Lambda | 81 |
| | [Escherichia | tgkefglgrdrriaiteaiqanielfsghk | | | |
| | virus Lambda] | hkpltarinsdnsvtlhswldryekilasr | | | |
| | | gikqktlinymskikairrglpdapledit | | | |
| | | tkeiaamlngyidegkaasaklirstlsda | | | |
| | | freaiaeghittnhvaatraaksevrrsrl | | | |
| | | tadeylkiyqaaesspcwlrlamelavvtg | | | |
| | | qrvgdlcemkwsdivdgylyveqsktgvki | | | |
| | | aiptalhidalgismketldkckeilgget | | | |
| | | iiastrreplssgtvsryfmrarkasglsf | | | |
| | | egdpptfhelrslsarlyekqisdkfaqhl | | | |
| | | lghksdtmasqyrddrgrewdkieik | | | |
| NP_700401 | Integrase protein | mgrkrapgnewmpkgvffrpsgyywkpggs | 329 | Lambda | 82 |
| | [Salmonella | teniapadatkaevwvayekkvegrknrit | | | |
| | phage ST64B] | ftqlwrkflasadyadlaprtqkdylahek | | | |
| | | yilavfgdaeakaikpehirrymdargqks | | | |
| | | rvqanhehssmsrvfrwsyqrgyvpgnpcv | | | |
| | | gvdkfpkpqrdryitdeeyraiynnatpav | | | |
| | | raameiaylcaarvsdvlkmnwnqilekgi | | | |
| | | fiqqgktgvkqikswtdrlrdaveicrewg | | | |
| | | eegpvirtmygerysykgfneawrkarkaa | | | |
| | | gddlglpldctfhdlkakgisdyegtakdk | | | |
| | | qkysghktesqvlvydrkvkmsptldrkr | | | |
| YP_009275635 | integrase family | maprprkegskdlppnlykktdsrsgvtyy | 367 | Lambda | 83 |
| | site-specific | ayrdpvsgrmfglgkdkaraireaieanht | | | |
| | recombinase | ealqptiadrlnsepsrpprlfddwlieye | | | |
| | [Pseudomonas | kiyaerglaaasvrntrmrlkrlrarfgtm | | | |
| | phage Phi2] | dirdigtidvagyfsemakegkaqmaramr | | | |
| | | sllrdvfmesmaagwtdknpvevtkaarvk | | | |
| | | ikrerltletwrliyaeakqpwlkramela | | | |
| | | vitgqrredlaamqfkdeqdgylqvvqskt | | | |
| | | gmrlristsiglavlgldlasvikscrgrv | | | |
| | | lsrymihhhrtisrakagqpimldtisaaf | | | |
| | | adardraakkhgldfgasppsfhemrslaa | | | |
| | | rlheeegrdaqrllghrsakmtdlyrdsrg | | | |
| | | aewidva | | | |
| AAB09182 | integrase | mavrkdtkngkwlaevyvngnasrkwfltk | 337 | Phages | 84 |
| | [Haemophilus virus | gdalrfynqakeqttsavdsvqvlessdlp | | | |
| | HP1] | alsfyvqewfdlhgktlsdgkarlaklknl | | | |
| | | csnlgdppanefnakifadyrkrrldgefs | | | |
| | | vnknnppkeatvnrehaylravfnelkslr | | | |
| | | kwttenpldgvrlfkeretelaflyerdiy | | | |
| | | rllaecdnsmpdlglivriclatgarwsea | | | |
| | | etltqsqvmpykitftntkskknrtvpisk | | | |
| | | elfdmlpkkrgrlfndayesfenavlraei | | | |
| | | elpkgqlthvlrhtfashfmmnggnilvlk | | | |
| | | eilghstiemtmryahfapshlesavkfnp | | | |
| | | lsnpaq | | | |
| AAG03003 | integrase | mkvsvnkrnpnskglqqlrlvyyygvvege | 405 | Phages | 85 |
| | [Salmonella enterica | dgkkrakrdyeplelylyenpktqaerqhn | | | |
| | subsp. enterica | kemlrqaeaarsarlveshsnkfqledrvk | | | |
| | serovar | lassfydyydkltaskesgsssnysiwisa | | | |
| | Typhimurium] | gkhlrsyhgraeltfeeidkkflegfrkyl | | | |
| | | leepltksqsklakntassyfnkvraalne | | | |
| | | afregiirdnpvqrvksvkaentqrtyltl | | | |
| | | devramtkaecrydvlkraflfscttglrw | | | |
| | | sdiqkltwkeieefqdghyriifkqaklln | | | |
| | | agnslvyldlpdsavklmgerqdkaervfk | | | |
| | | glkyssytnvallhwamlagvqkhvtfhvg | | | |
| | | rhtfavaqlnrgvdiyslsrllghselrtt | | | |
| | | eiyadilesrrvtamrgfpdifedkvqesg | | | |
| | | tccphcgksvlnktl | | | |
| NP_046786 | Int [Escherichia | maikklddgryevdirptgmgkrirrkfdk | 337 | Phages | 86 |
| | virus | kseavafekytlynhhnkewlskptdkrrl | | | |
| | P2] | seltqiwwdlkgkheehgksnlgkieiftk | | | |
| | | itndpcafqitkslisqycatrrsqgikps | | | |
| | | sinrdltcisgmftalieaelffgehpirg | | | |
| | | tkrlkeekpetgyltqeeialllaaldgdn | | | |
| | | kkiailclstgarwgeaarlkaeniihnrv | | | |
| | | tfvktktnkprtvpiseavakmiadnkrgf | | | |
| | | lfpdadyprfrrtmkaikpdlpmgqathal | | | |
| | | rhsfathfminggsiitlqrilghtrieqt | | | |
| | | mvyahfapeylqdaislnplrggteaesvh | | | |
| | | tvstve | | | |
| NP_059584 | Int [Salmonella virus | mslfrrgetwyasftlpngkrfkqslgtkd | 387 | Phages | 87 |
| | P22] | krqatelhdklkaeawrvsklgetpdmtfe | | | |
| | | gacvrwleekahkksldddksrigfwlqhf | | | |
| | | agmqlkditetkiysaiqkitnrrheenwk | | | |
| | | lmdeacrkngkqppvfkpkpaavatkathl | | | |
| | | sfikallraaerewkmldkapiikvpqpkn | | | |
| | | krirwlepheakrlidecqeplksvvefal | | | |
| | | stglrrsniinlewqqidmqrkvawihpeq | | | |
| | | sksnhaigvalndtacrvlkkqignhhkwv | | | |
| | | fvykesstkpdgtkspwrkmrydantawra | | | |
| | | alkragiedfrfhdlrhtwaswlvqagvpi | | | |
| | | svlqemggwesiemvrryahlapnhlteha | | | |
| | | rqidsifgtsvpnmshsknkegtnnt | | | |
| NP_459869 | putative Fels-1 | mtlldaggimakpayptgvekhgdklricf | 441 | Phages | 88 |
| | prophage integrase | hykgrrvrenlgvpdtpknrkvagelrasv | | | |
| | [Salmonella phage | cfaikvgtfdyaaqfpdspnlklfgivnke | | | |
| | Fels-1] | itvaeladkwlklkemeiskntmlryesii | | | |
| | | kisvsllggrvlassvtqedllffrkelmt | | | |
| | | ghhitrpgrelapkgrsvatvnsylgvvsg | | | |
| | | lfqfaarngyipqnpfngitmlkrakaepd | | | |
| | | plsreefarlidachhqqiknlwslavytg | | | |
| | | mrhgelcalawedidlkagtlivrrnytqa | | | |
| | | keftlpktqagtdrvihlvqpaidalksqa | | | |
| | | sftklskqhkievklreygrtkthsctfvf | | | |
| | | npqitdrsgkskahyaapslnriwesalrr | | | |
| | | aglrhrkayqsrhtyacwalaaganpnfia | | | |
| | | sqmghsnaqmvytvygawmadnnqsqvdil | | | |
| | | nqqlastapgvpqkdnmlnfi | | | |
| NP_536628 | Int [Vibrio virus | msvrnlkdgskkpwlcecypqgregkrvrk | 345 | Phages | 89 |
| | K139] | rfatkgeatayenfimrevddkpwmgskpd | | | |
| | | nrrlselletwwqvhghtiksgkvvyrkta | | | |
| | | ltikelgdpiastftskqylafrasrvshf | | | |
| | | nkenkslsptyqnfqlnllsgmfsrlikyk | | | |
| | | qwnlpnplddiepikvnqralayldkadiq | | | |
| | | pflqrlggfesdgrsvsipeivliakicla | | | |
| | | tgarisealslersqisefkltfvetkgkr | | | |
| | | irsvpisenlykeimlasssstkifsttyg | | | |
| | | sahryikkalpdyvpegqathvlrhtfath | | | |
| | | fmmnrgdililqrilghqkieqtmayahfs | | | |
| | | pdhliqavqlnplen | | | |
| NP_599058 | integrase | mslfrrgeiwyasftlpngkrfkqslgtkd | 387 | Phages | 90 |
| | [Enterobacteria | krqatelhdklkaeawrvsklgeipditfe | | | |
| | phage SfV] | eacvrwleekahqksldddksrigfwlqhf | | | |
| | | agmqlrditeskiysaiqkmtnrrheenwr | | | |
| | | lraeacrkkgkpvpeytpkpasvatkathl | | | |
| | | sfikallraaerewkmldkapiikvpqpkn | | | |
| | | krirwlepheaqrlidecpeplkswefala | | | |
| | | tglrrsniinlewqqidmqrrvawinpees | | | |
| | | ksnraigvalndtacrvlkkqignhhrwvf | | | |
| | | vykesctkpdgtkaptvremrydantawka | | | |
| | | alrragiddfrfhdlrhtwaswlgqagvpl | | | |
| | | svlqemggwesiemvrryahlapnhlteha | | | |
| | | rqidsilnpsvpnssqsknkegtndv | | | |
| NP_996675 | integrase | matyqkrgktwqysisrtkqglprltkggf | 374 | Phages | 91 |
| | [Lactococcus | stksdaqaeamdiesklkkgfivdpikqei | | | |
| | phage phiLC3] | seyfkdwmelytknaidemtykgyeqtlky | | | |
| | | lktympnvliseitassyqralnkfaetha | | | |
| | | kastkgfhtrvrasiqplieegrlqkdfit | | | |
| | | travvkgngndkaeqdkfvnfdeykqlvdy | | | |
| | | ffnrlnpnyssptmlfiisitgmraseafg | | | |
| | | lvwddidfnnntikcrrtwnyrnkvggfkk | | | |
| | | pktdagirdividdesmqllkdfreqqktl | | | |
| | | feslgikpihdfvcyhpyrkiitlsalqnt | | | |
| | | lehalkklkistpltvhglrhthasvllyh | | | |
| | | gvdimtvskrlghasvaitqqtyihiikel | | | |
| | | enkdkdkiielllel | | | |
| WP_016065986 | MULTISPECIES: | mairklpeggwlselypngakgkrirkkfa | 345 | Phages | 92 |
| | integrase | tkgealayeqhavqlpwneeqtdrrtlkdl | | | |
| | [Erwiniaceae] | itswysahgitlkdgekrqlamlhafecmg | | | |
| | | eplavdfdaqmfsryrerrlkgdfarssrv | | | |
| | | kevsprtlnlelayfravfnelgrlgewkg | | | |
| | | enplrhirpfrteesemawlthsqiahlla | | | |
| | | ecrnsdqadletvvkiclatgarwseaegl | | | |
| | | kksqiskykityiktkgrknrtvpitesiy | | | |
| | | riipenktgrlfadcygaffsalertgiel | | | |
| | | pagqlthvlrhtfashfmmnggnllvlqrv | | | |
| | | lghtdikmtmryahfapdhleeaaklnpla | | | |
| | | qsgdemaiemanvgn | | | |
| YP_004934132 | phage integrase | msiklrggtwhcdfvapdgsrvrrsletsd | 386 | Phages | 93 |
| | family protein | krqaqelhdrlkaeawrvknlgespkklfk | | | |
| | [Escherichia | eacirwlreksdkksidddksiisfwmlhf | | | |
| | phage HK75] | retilsditsekimeavdgmenrrhrlnwe | | | |
| | | msrdrclrlgkpvpeykpklaskgtktrhl | | | |
| | | ailrailnmavewgwldrapkistprvkng | | | |
| | | rirwlteeeskrlfaeiaphffpwmfaitt | | | |
| | | glrrsnvtdlewsqvdldkkmawmhpdetk | | | |
| | | agnaigvplnetacqilrkqqglhkrwvfv | | | |
| | | htkpayrsdgtktasvrkmrtdsnkawkga | | | |
| | | lkragisnfrfhdlrhtwaswlvqsgvsll | | | |
| | | alkemggwetlemvqryahlsaghltehas | | | |
| | | kidaiisrngtntaqeenvvylnar | | | |
| YP_005087193 | unnamed protein | mprpslpvgahgrisrtklpdgrwraacrf | 412 | Phages | 94 |
| | product | fdadgvtrqvvrytpptvdrdktgaaaera | | | |
| | [Rhodococcus | lvdalkgrsttgdlsadsrvselwmayraq | | | |
| | phage REQ3] | leeknrsqstlqdydrmaakildglgnlrv | | | |
| | | reattqrldtfvreiatrqgagtgkkakti | | | |
| | | lsgmfriavrygavqanpvrevtdlgagrk | | | |
| | | kraksmdrellvqlladvrgseapcpvvls | | | |
| | | eaqikrgvkttskagqvpsvaqfcqaadla | | | |
| | | dlivmfaatgarigevlgirwedvdlkkrt | | | |
| | | vaiagkvirvkgdglvredstktesglrql | | | |
| | | plpgfavemlekrlvdrtgpmvfpskvgtl | | | |
| | | rdpdtvqrqwrqvraaldlewvtthtfrkt | | | |
| | | vatilddegltarqaadhlghaqvsmtqdv | | | |
| | | ylgrgrthsaaaaaldaavakr | | | |
| YP_008409003 | integrase | mptvrkrtrsdgtpcylvqyrfggrgskqg | 375 | Phages | 95 |
| | [Mycobacterium | altfddpkaaeafaaavtahgaaralemyg | | | |
| | phage Bobi] | idpsprrtdgrskgmtvaewvrhhidhltg | | | |
| | | veqytldkyeqylanditphlgdiplskls | | | |
| | | eddiarwvkvmettggrdgnghapktlmky | | | |
| | | gflsgalnaavprylstnpasgrrlprgna | | | |
| | | edddeirmlthaefdrlrdavtphwklmvq | | | |
| | | fmvstglrwgevsalqpkhvdletstirvr | | | |
| | | qawkyssagyvlgppktkrsrrtvdvparl | | | |
| | | lerldlsnefvfvntdggpvrypgflrrvw | | | |
| | | npavekaglvprptphdlrhtyaswqltgg | | | |
| | | tpvtivsrqlghesiqitvdtytdvdrtss | | | |
| | | rvaaefmdgllgdf | | | |
| YP_009002695 | integrase Y-int | masirtrsrkdgstytqvryrlngeetsts | 365 | Phages | 96 |
| | [Mycobacterium | fddvghavefkrmvdqlgaakaleviettd | | | |
| | phage Validus] | aasqhytlgewldhylrhktgvekstlydy | | | |
| | | rkmvekdiapalgaiplaaltaedvakwvq | | | |
| | | glaeaglagktisnkhgflssalnvavtrg | | | |
| | | hiaanpatagaglievprteraemvflsre | | | |
| | | qyaklhdnmplrwqplveflvasgarwgev | | | |
| | | talrpsdvnradgtvrisrawkrtyasggy | | | |
| | | algapktersrrtinvdasvldkldyshew | | | |
| | | lfvngrgapvrghnfhenhwqpaikragld | | | |
| | | vkprihdlrhtcaswliaagvplpaiqqhl | | | |
| | | ghesikvtigvyghldrshgktvaaaiaaq | | | |
| | | ldpgr | | | |
| YP_009032437 | integrase | masirsvsrkdgttftqvryrlngkqtsts | 366 | Phages | 97 |
| | [Mycobacterium | fddgahavefkrmveqlgaakalevlettd | | | |
| | phage ZoeJ] | aasmftlagwlkhyldhktgvekstiydyr | | | |
| | | kmvekditpvlgaiplaaltaedvakwvqg | | | |
| | | ladkglagktiankhgflssalnvaasagh | | | |
| | | ikanpavggaglvavprteraemvfltadq | | | |
| | | yaklhdnmplrwqplveflvasgarwgevt | | | |
| | | alrpsdvnraegtvrisrawkrtyarggye | | | |
| | | lgapktnksrrtinvdtavldrldysgew | | | |
| | | lftnvrggpvrghnf | | | |
| | | henhwqpalkkagldgldvkprihdlrhtc | | | |
| | | aswliaagvplpaiqqhlghesiqvtigvy | | | |
| | | ghldrssgrtvaaaiaaalgr | | | |
| YP_009195219 | integrase | mkghfykpnckcpgkktkkcscgatwsyii | 407 | Phages | 98 |
| | [Paenibacillus | dvginpntgkrkqkkkggfktkteaqeaaa | | | |
| | phage | llvaelsqgtyveeknntfeeyakewlsey | | | |
| | HB10c2] | qatgtvkistvrirkkgiklllpylaklri | | | |
| | | siitakqyqhalldlhdkgysnntivsahq | | | |
| | | tgrmifqraielkiikndptssavipkrqr | | | |
| | | tiedletekeipkymekeelalflqtakek | | | |
| | | gldrdyaifltlaytgmrvgelcalkwsdi | | | |
| | | dfseqtvsitktyynpnnniknytlltpkt | | | |
| | | ksskrviivdkkvldeleqlqaeqkrikmf | | | |
| | | frktyhdknfvfsqqgeenagfptypklva | | | |
| | | lrmtrllklaglntkltphslrhthtslla | | | |
| | | earvsleqimqrlghrsdettkniylhvtk | | | |
| | | pkkkeasqkfaelmssf | | | |
| YP_009304294 | integrase | masihtrtladgtdsyrvswrhngrqrrls | 359 | Phages | 99 |
| | [Gordonia | feniqaatthklnlekfghdramqilgvie | | | |
| | phage Lucky 10] | thrdettltqtlehhinsltgvepgtirry | | | |
| | | hsylrndfadigqlpvsgisetviaswite | | | |
| | | lakknsgktiankhgllsaalaravregrl | | | |
| | | tanpcdhtrlprkdpvddpvfldrdqfdel | | | |
| | | aaampehwrplatwlvmtgmrfseataltv | | | |
| | | gditptstggvvriskawkwtgttekrlsy | | | |
| | | pksragrrtinvpaqaiqlldldrpktrll | | | |
| | | ftnmddrvtysrfydggwkpamqktawhas | | | |
| | | phdlrhtcaswmiaagvplpviqahlghes | | | |
| | | itvtigvyghldrsshesaaaaigqmfg | | | |
| AAM88709 | putative | mskerhahedalnetefqklldgahlltpp | 224 | PhiCh1 | 100 |
| | site-specific | anleatfvitmsgklgmrigeiahmkrtwv | | | |
| | recombinase Int1 | kpdqglievpshepcekgrdgglcgycrrq | | | |
| | [Natrialba | anrtyqndpenrdldellksywepkteaae | | | |
| | phage PhiCh1] | ravpyefdedvedvvssffeyyyevplsvn | | | |
| | | tcrrrvkdaaeasdlnrrvyphalrataas | | | |
| | | thayeglniasmkammgwaklstaekyiri | | | |
| | | sggrtkralleiyg | | | |
| WP_081461325 | site-specific | mserefqlllegaaslrdpyaqqarfvilv | 216 | PhiCh1 | 101 |
| | integrase | agrlgmrageiahmdrswidwrnqmiwprh | | | |
| | [Halalkalicoccus | dpctkargeagpcgyckrlaeqaadhnpel | | | |
| | jeotgali] | syeaalarawtpktdsaarsipfdfdprtd | | | |
| | | lvierfferyekfphskqavnrrvnkaaev | | | |
| | | tdeldedsiyphclrataaty | | | |
| | | hasrglsalplqsmlgwsdlstsqkyvrrs | | | |
| | | geataralrtvhrq | | | |
| WP_081927589 | site-specific | mvatreralserefelllegagrigdtqrr | 223 | PhiCh1 | 102 |
| | integrase | letraaillggrlglrpgetthlskswvdl | | | |
| | [Halobellus | erqmiqippqenctkgrdggicgycrqavk | | | |
| | rufus] | qrldhnpntdfqsfadrywlpkteaasrtv | | | |
| | | pyhfsyrvrvavelllnehsgwpysfstlq | | | |
| | | rrletalerspelsndatslhglrataasy | | | |
| | | hagrgldlpalramfgwedittarqylnvd | | | |
| | | gamtrraldsihq | | | |
| WP_082256404 | integrase | maptrekslserefelllegagridepvqr | 222 | PhiCh1 | 103 |
| | [Haloferax | lesraailiggrlglrpgetthlssswidh | | | |
| | sp. ATB1] | erqmiripehhactkgrdgglcgycrqaie | | | |
| | | qrlrhdpdsrfedfadlywlpktdaaartv | | | |
| | | pfhfsyrvrvaidllitehggwpysfstlq | | | |
| | | rrlntaldlaprlsrnatslhglrataasy | | | |
| | | hasrglelaalramfgwediatarqylnvd | | | |
| | | gamtrralnnih | | | |
| YP_008059154 | integrase | mrkeirenrkgrytredalndrefqllleg | 233 | PhiCh1 | 104 |
| | [Halovirus | aremehyysqqarfiilvagrlgmrkgeit | | | |
| | HCTV-5] | hiqekwvdwrkdmieiprfepcdkgkngga | | | |
| | | cgyckqqakqaveyneeadieeeirckwep | | | |
| | | kteaaarkipfgfdprtslilerffdryde | | | |
| | | fcwsaqaitrrvkkaaklakeldeeeiyph | | | |
| | | clrataatyhasrglemvplqamfgwaqps | | | |
| | | tamnyiqnsgentaralhmvhsq | | | |
| CAA09137 | hypothetical | maevgnhlgkignhlnpevetnimpildid | 439 | pNOB | 105 |
| | protein | kltneqkirlftyvteekgityeqlgiska | | | |
| | (plasmid) | tgwrykkglreipkeimekalqflapdeia | | | |
| | [Sulfolobus | rwygkkiekadindllkvintavedlqfrs | | | |
| | sp. | llfmmlnrflgeyvkqntnsyavteedlkl | | | |
| | NOB8H2] | fekileqkskatkeerlrhikyamkdlgfs | | | |
| | | lspeslkeyivelaaeegpnvarhrantlk | | | |
| | | lfikevvmsrnpilgqilynsfkvpkvdyk | | | |
| | | yspppisldllkkifqsidhlgaktfflil | | | |
| | | aetglrvgevysltleqvdlengiiklmks | | | |
| | | satkrayisflhketiewikknylpfredf | | | |
| | | iskyekavqqiggdvekwrmkffpfqladl | | | |
| | | raevkegmrkvgkefrlydlrsffasymak | | | |
| | | sgvspfiinvlqgrmapgqfkilqqhyfvi | | | |
| | | sdielkkiyeekapklls | | | |
| WP_010979387 | integrase | mivdvsslseeqkikivetvlqkgisykel | 413 | pNOB | 106 |
| | [Sulfurisphaera | gidrvtwwryknkkrkipdevvqkaaeylt | | | |
| | tokodaii] | pdelvqltysidiskigineaigvivkatk | | | |
| | | dpefrefflsllqrnlgefikaasysypit | | | |
| | | qedlqmfkklienkakntfedywryinria | | | |
| | | kdnnyvispdkikdyileqfdesphrarqm | | | |
| | | atvlklfikeivrskdpilaqilyhsfsip | | | |
| | | rpktkykpavlsldllkkvfseiqelgakt | | | |
| | | yfliaaetglrtgelfylsvnqvdlqhrii | | | |
| | | klfkenetkrayiaflhretakwieenylp | | | |
| | | yrenyirrhwggvkaigqdiekwkmkffpm | | | |
| | | nedkmraeikaamqrggkvfrlydlrafwa | | | |
| | | symikqgvspmivnilqgraapnqfrilqe | | | |
| | | hylpfseeelreiyekyapkllt | | | |
| WP_012548831 | helix-turn-helix | mlinvskldeqqrkriikklveklglsqaa | 419 | pNOB | 107 |
| | domain-containing | kmlgvgrstlyryvnsdrnipldivrkaae | | | |
| | protein | mlaqdelsdaiyglkvvevdattalsvwka | | | |
| | [Acidianus | mkdekfrnffvsilyqylgdylksasstyi | | | |
| | hospitalis] | vteedvkkfekllqgkskstidmrmrylri | | | |
| | | altklgyelspdsirdliaelsedssniar | | | |
| | | htanslklfiktvvkeknlqlaqllynsfk | | | |
| | | vpkskykykpqpltletlrrifdnidhlga | | | |
| | | kafflllsesglrvgevyslkvdqldlenr | | | |
| | | iikvmkesetkrayisfihtetrkwlqevy | | | |
| | | fpyreefvrtyefavkqigadveawkqklf | | | |
| | | pfqladlrssikegmrkvlgkefrlydlrs | | | |
| | | ffasylikngvspmivnilqgrappaqfqi | | | |
| | | lqnhyfvmseielqkvfdekgpkllspk | | | |
| WP_012735688 | integrase | Mrhskliyinyvdgyllimdttkldddkk | 433 | pNOB | 108 |
| | [Sulfolobus | lkilekaiekfgkayiaq | | | |
| | islandicus] | kcgvsrqtiyrylkreiqsipdefiqcvsn | | | |
| | | flsieelgdivyglrtvevdenialsvivk | | | |
| | | mkrdpnfrafflslmkqflgeyiqdastsy | | | |
| | | vitkndvdrflnyiksksnttyktfknyfv | | | |
| | | ktiaelnytltpeavkdyitkemtiskgra | | | |
| | | shiskilklfikeiiipknsslgrelynsf | | | |
| | | ktikvekeyspesltledlkrvfttiehig | | | |
| | | akafflllaetglrineilklnidqidlek | | | |
| | | riiyvnkisaskrayitflhentakwlket | | | |
| | | ylpyreefinkyekklrnininveawknrl | | | |
| | | fpineynmrkeikeamkkvlsrefrlydlr | | | |
| | | sffasymikqgvspmivnllqgrappqqfq | | | |
| | | ilqnhyfvvsdielqqyydkyaprll | | | |
| WP_052885762 | hypothetical protein | mirsgrrrvgdgllcsmlrlltpeelqsll | 385 | pNOB | 109 |
| | [Vulcanisaeta | rgwvperraslsdalrviitaredptfreq | | | |
| | distributa] | flallsrylgdyvqslgrawhvtqedieaf | | | |
| | | ikakrlkgvgektlndelryirraleeldw | | | |
| | | vltpegiteflgglaeeespyvvrhvtvsl | | | |
| | | ksliktvlkprdpglfavlynsfttikprn | | | |
| | | hnktklptleelrqvlskiesieaktyfii | | | |
| | | laetglrpsepflvsmddvdlehgmlrigk | | | |
| | | itetkrtfiaflqpktlefikaqymprrdw | | | |
| | | lvrnrleaikadylgvkpsvedwarkfmpf | | | |
| | | drdrlrreikeaarqvlgrdfelyelrkff | | | |
| | | atwmisrgvpesivntlqgrappsehrili | | | |
| | | ehywsprheelmwylrhapcllch | | | |
| WPJ166797986 | site-specific | mdpdlirveaipqdvrrkvleyvtgvkgig | 426 | pNOB | 110 |
| | integrase | psdlgynktymyrvrhgmvpisdglikall | | | |
| | [Caldivirga | rfidideyarlvgsapplveatpddivrvv | | | |
| | sp. MU80] | kkalvdksfrnllfdmlrqafgdefreyra | | | |
| | | swtvkeadieefvrakrlkglsgrtirdev | | | |
| | | ryirlalselnwvlepegireyiaglaeeg | | | |
| | | eyniarhvsvglksilktvlkprdpalfrl | | | |
| | | lydsftvykhkasthvklptleqlrliwar | | | |
| | | lpsvearfyftvlaecglrpsepflasidd | | | |
| | | ldlehgvirigkvtetkrsfvaflrpefad | | | |
| | | wvresylparealikakldivradylgvna | | | |
| | | naedwarrlipfdrgrlrreikeaakqvlg | | | |
| | | relelyelrkffatwmisqgvpesivntlq | | | |
| | | grappsefrilvehywspxheelrqwylry | | | |
| | | aprvcc | | | |
| WP_081228025 | hypothetical protein | mkpmvdceliniekigneervriinyvmek | 431 | pNOB | 111 |
| | [Vulcanisaeta | kgvkardlgvtlnlismirsgkrrvtedll | | | |
| | sp. | cralkflsneelakllgqipelepasisdl | | | |
| | EB80] | vrvvararadpeyrdlllsyldrylgdyvr | | | |
| | | amgnkwvvteqdieefikakrlegvtektl | | | |
| | | rdythylremlaelnwnltpdgireylsgl | | | |
| | | aeegeehvlhhlttalksllktileprdpf | | | |
| | | lfgllyhafktykaksnnriklptidqlrq | | | |
| | | iwqqlptietrfyfallaetglrpgepfll | | | |
| | | siddldlehgmlrigkvtetkrafvaflrp | | | |
| | | eflewvktnylphreawivrmaklwessnl | | | |
| | | fitqeviekakrklipfdqsrlrreikdta | | | |
| | | rqvlgrefelyelrkffathmisqgvpesi | | | |
| | | vntlqgrappsefrvlvehywsprheelrg | | | |
| | | wylkyaprvccd | | | |
| YP_008369965 | integrase (plasmid) | mltdvtklddeqrrrilkklveklglaqta | 419 | pNOB | 112 |
| | [Saccharolobus | klleigrstlyryvntnqnipleivrkaad | | | |
| | solfataricus | mltpdelsdviyglkvvevdattalsvvik | | | |
| | P2] | amkdekfrnffvsvlyqylgeylkntssty | | | |
| | | ivtgedvkrfekslqgktkstidmrmryli | | | |
| | | palirlgyelspdgirdllaelseessnia | | | |
| | | rhtanslklfikavireknlqlaqllynsf | | | |
| | | kvpksrykyrpqplsletirdifdnishlg | | | |
| | | arafflllaesglrvgevyslkldqldlen | | | |
| | | rvikvmketetkrayvsfihietrkwlqei | | | |
| | | yfpyreefirtyehavkqigadvevwkqkl | | | |
| | | fpfqladlrasikegmrkvlgkefrlydlr | | | |
| | | sffasymikngvspmivnllqgrapptqfq | | | |
| | | ilqnhyfvmseielqrifdekgpkllslk | | | |
| YP_138392 | integrase (plasmid) | mlidvtkldeeqrkrilkklidklgltlaa | 419 | pNOB | 113 |
| | [Sulfolobus | kmlgvgrstlyryvntnqsiplevvkkate | | | |
| | islandicus] | mlapdelsdaiyglkvvevdattalsvvik | | | |
| | | aikdekfrnffvsilyqylgdylksassty | | | |
| | | ivteedvkkfekslqgkskstidmrirylr | | | |
| | | malirlsyelspdgirdllaelseessnia | | | |
| | | rhtanslklfiktvvkeknlqlaqllynsf | | | |
| | | kvpkskykykpqplsvdtlrkifdsidhlg | | | |
| | | akafflllaesglrvgevyslkmdqldlen | | | |
| | | riikvmkesetkrayisfvhketkewlqgv | | | |
| | | yfpyreefirtyehvvkqigadveawkqkl | | | |
| | | fpfqladlrasikegmkkvlgkefrlydlr | | | |
| | | sffasylikngvspmivnilqgrappaqfq | | | |
| | | ilqnhyfvmseielqkifdekgpkllspk | | | |
| WP_013683375 | hypothetical protein | mrglykeraaeafneavldydkykeefkew | 291 | pTN3 | 114 |
| | [Archaeoglobus | lfkevsketaeqylrdleqtiagkkindph | | | |
| | veneficus] | elyniykdypqrhhrkairtfmrfliksgi | | | |
| | | rkkselmdfqavidipgtqprppeeafttd | | | |
| | | ekiiealnspkvkkderrqilirllaytgl | | | |
| | | rlrealellrtfdknklefhgnyaryptye | | | |
| | | lkskagtkrtyyaympadfarqlkridike | | | |
| | | ttvkgakladriilpeqlrkwhtnflkrki | | | |
| | | kekklqlgvtaetlinfiqgrvgkavidry | | | |
| | | yldlvedadelytkiadefpf | | | |
| WP_013748767 | integrase | mvgprgfeprtstlseklndlwsfykiqfs | 287 | pTN3 | 115 |
| | [Pyrococcus sp. | ewlsgqitevvrkdyikaldkffdrheivt | | | |
| | NA2] | yqdleralkfenytdrlvkglrkfvtfleee | | | |
| | | hildfrraddlrriiklrretrirdvfisde | | | |
| | | elriayekvkqkelvkvvlfellvfsgirls | | | |
| | | havqllnsfdesklfrindkiaryplfaisr | | | |
| | | gkkrgfwayapvelfekimsigrqninykta | | | |
| | | qdwvtygkvsantirkwhytfmirqgvpaei | | | |
| | | adfiqgrasrtvgpthylnktiladewysvi | | | |
| | | vdelkkvleg | | | |
| WP_048053722 | hypothetical protein | makkyiplldkylwgkkantpeelrkiies | 292 | pTN3 | 116 |
| | [Thermococcus | ipptkkgnpnrhaylairsyinflvdtgri | | | |
| | kodakarensis] | rkseaidfkavipniktnaraesakvitse | | | |
| | | diremfsqlkgknetilrarklylkllaft | | | |
| | | glrgdevrelmnqfdprvveetfkafglpe | | | |
| | | ewrkkiavydmervklptrrhgtkrgyvav | | | |
| | | fpaelvrelewfastgykltadnsdkhklf | | | |
| | | rdytkvkdlallrkfwqnfmndnvmstvpn | | | |
| | | ppadafhlieflqgrapktvggrnyrwnvm | | | |
| | | avriyyymvdrlkeelgilel | | | |
| WP_048148949 | hypothetical protein | mnprpadyksvialktlnevwnhekkafle | 286 | pTN3 | 117 |
| | [Palaeococcus | wlslkigrertvkdyynalkvmfkdyevrp | | | |
| | ferrophilus] | tkksiknaidalgnkkryvyglrnflkylt | | | |
| | | ekelinedfskmlqgaakakksgvrevhln | | | |
| | | dheiteawqhvknrreeaqmlfkamvfsgi | | | |
| | | rlaqlirmfktydparlqfplegiarypik | | | |
| | | disegkkkgfwayfpadlvpelrrfsaket | | | |
| | | tawkwvrygrvsansirkwhytflirkgvp | | | |
| | | adladfiqgreaetvgarhylnktlladew | | | |
| | | ystvvddlkkvlegek | | | |
| WP_070105199 | hypothetical protein | mkdyisalerffgrhtirdikglkvslqqe | 247 | pTN3 | 118 |
| | [Thermococcus | nynekivkglrnfvnflldeglinegtaal | | | |
| | kodakarensis] | fkkpltfkrgtprqvfisneelreayielt | | | |
| | | khygkeaevlfkllaftglrlkhivkmlnt | | | |
| | | ydpqklvivnekvarypmaehgkgtkrafw | | | |
| | | aympadfarslermsityfqaqprttykrv | | | |
| | | sastvrkwfstflaqrkvsmevidfiqgra | | | |
| | | prsvlerhylnltvladeayakvvddlrkv | | | |
| | | legqthd | | | |
| WP_084063640 | hypothetical protein | mrssaarqftssiseiesnnglirypeeak | 327 | pTN3 | 119 |
| | [Geoglobus | gsklhqkyngynerikfedidyedfelfwt | | | |
| | acetivorans] | aerkmktskgrvkrlynvlrkvlsgkvine | | | |
| | | eslregfhkttnkkdyvnavrvlleylkvr | | | |
| | | klmprevvqeileqpfltpirskrrgiylk | | | |
| | | deeirqayewlkekwkdkdtellfkllvfs | | | |
| | | girldhaldllynfdprklefkgrvarypl | | | |
| | | tnisneiksgeyafmpaefarklkkikkkl | | | |
| | | nyqtwenrinvkrwrgdekykksrvdanai | | | |
| | | rkwfgnfclshdvsesateyfmghaikgmg | | | |
| | | gkayfdlrdklswreyekivdkfpipp | | | |
| YP_005271232 | unnamed protein | mnemginksqffndtarwvflgeempeiiv | 318 | pTN3 | 120 |
| | product | klewcggrdlnpghrlgrslslnemwvayr | | | |
| | [Thermococcus | aefekallaevaettakdylsalnrffgah | | | |
| | prieurii | kikttedlrnsylkegqkrnlgkglrkfft | | | |
| | virus 1] | flyqhdaisfelyqklkniiklkptkasgk | | | |
| | | fittgelleaydyffkhgrpeelllffila | | | |
| | | ysgirlrhavqllnsfsrdkliyhenfaky | | | |
| | | plfkhegtkvvyyaymprelaeelfqsgyt | | | |
| | | edmarkylrygkvsastirkwfstflvskg | | | |
| | | vppaavnyiqgrkpknvldayyvqleklad | | | |
| | | eaysrvlpdlkkvledge | | | |
| YP_008619357 | SSV1-like integrase | mvksggvyvhsqatgeeqagarkrrrprrl | 455 | pTN3 | 121 |
| | (plasmid) | sprlyitlppeiyrkakerwdnvsriiasl | | | |
| | [Thermococcus | levalaedltveevvtavtllrsgalvvns | | | |
| | nautili] | pssagvaepgqrrwtqdalfspneglsrqn | | | |
| | | dnkeepsadnvftgkalidstakihygrdr | | | |
| | | qkyiewvkrrtpsmadkyislldkylwgkk | | | |
| | | antpedlrriveaipptrggfpnrhaymal | | | |
| | | rsyinflvdtgklrkseaidfkavipnvkt | | | |
| | | naraesakvitvediremfnqlkgknetil | | | |
| | | rarklylkllaftglrgdevrelmnqfdpr | | | |
| | | videtfkafglpeeykekiavydmervkik | | | |
| | | trrsqtkrgyvavfpaelvpelewffstgy | | | |
| | | kltadnsdkhklfrdskevkdlallrkfwq | | | |
| | | nfmndnvmstvpnppadtwhlieflqgrap | | | |
| | | knvggrnyrwnvknavriyyymvdklkeel | | | |
| | | gilel | | | |
| BAA75171 | shufflon-specific | mpsprirkmslsraldkylktvsvhkkghq | 384 | Shufflon | 122 |
| | recombinase | qefyrsnvikrypialrnmdeittvdiaty | | | |
| | (plasmid) [Shigella | rdvrlaeinprtgkpitgntwlelallssl | | | |
| | sonnei] | fniarvewgtcrmpvelvrkpkvssgrdrr | | | |
| | | ltsseerrlsryfreknlmlyvifhlalet | | | |
| | | amrqgeilalrwehidlrhgvahlpetkng | | | |
| | | hsrdvplsrrarnflqmmpvnlhgnvfdyt | | | |
| | | asgfknawriatqrlriedlhfhdlrheai | | | |
| | | srffelgslnvmeiaaisghrsmnmlkryt | | | |
| | | hlrawqlvskldarrrqtqkvaawfvpypa | | | |
| | | hittineengqkahrieigdfdnlhvtatt | | | |
| | | keeavhrasevllrtlaiaaqkgervpspg | | | |
| | | alpvndpdyimicplnpgstpl | | | |
| BAB91676 | shufflon-specific | mpsprirkmslsraldkylktvsvhkkgh | 384 | Shufflon | 123 |
| | DNA reconbinase | qqefyrsnvikrypialrnmdeittvdiat | | | |
| | (plasmid) | yrdvrlaeinprtgkpitgntvrlelalls | | | |
| | [Salmonella | slfniarvewgtcrtnpvelvrkpkvssgr | | | |
| | enterica | drrltsseerrlsryfreknlmlyvifhla | | | |
| | subsp. enterica | letamrqgeilalrwehidlrhgvahlpet | | | |
| | serovar | knghsrdvplsrrarnflqmmpvnlhg | | | |
| | Typhimurium] | nvfdytasgfknawriatqrlriedlhfhd | | | |
| | | lrheaisrffelgslnvmeiaaisghrsmn | | | |
| | | mlkrythlrawqlvskldarrrqtqkvaa | | | |
| | | wfvpypahittideengqkahrieigdfdn | | | |
| | | lhvtattkeeavhrasevllrtlaiaaqkg | | | |
| | | ervpspgalpvndpdyimicplnpgstpl | | | |
| CAR09669 | shufflon-specific | mfrkikirkmtlnraldkylktvsihkkgh | 374 | Shufflon | 124 |
| | DNA recombinase | lqefyrvnvikrhpmaerymdeittvdiat | | | |
| | [Escherichia coli | yrdqrlaqinprtgrqitgntvrlelalls | | | |
| | ED1a] | slfniasvewgtcrmnpvelvrkpkissgr | | | |
| | | drrltsgeerrlsryffdknqqlyvifhla | | | |
| | | letamrqgeiltlrwehldlqhgvahlpet | | | |
| | | knglprdvplsrkamylqilpqqingnvfs | | | |
| | | ytssgfksawrtalldlkienlhfhdlrhe | | | |
| | | aisrffelgtlnvmevaaisghrslnmlkr | | | |
| | | ythlrayqlvskldtkrkqtckiapyfvpy | | | |
| | | patvgnrnglfivtlhdfdletraetrela | | | |
| | | ishasvlllrtlaqaaqrgervptpgelpa | | | |
| | | nidarvmicplts | | | |
| WP_025211037 | site-specific | mpsprfrirkmtlsraldkylktvsvhkkg | 385 | Shufflon | 125 |
| | integrase | hlqefyranvirrypiaqrfmdeittvdia | | | |
| | [Escherichia | ayrdmrlaeinprtgkaitgntvrlelall | | | |
| | coli] | ssmyniarvewgtcrdnpvelvrkprvspg | | | |
| | | rerrltsseerrlsryffernmslyvafhl | | | |
| | | aletamrqgeilslrwehidlrhgvahlpe | | | |
| | | tknghsrdvplsrramflqmlpvalhggvf | | | |
| | | sytssgfksawriatqtlriedlhfhdlrh | | | |
| | | eaisrffelgslnvmeiaaisghrsmnmlk | | | |
| | | rythlrawqlvskldarrrqtqkvaawfvp | | | |
| | | ypghittddgqtvridicdfddlsvtaatr | | | |
| | | eealsrasevllrtlaiaaqkgervpapga | | | |
| | | lpvndpafvmvcplnpqgaltaqv | | | |
| WP_050303304 | site-specific | msrpqrikkmslskaldkyyatvsvhkrgh | 383 | Shufflon | 126 |
| | integrase | qqefyrvrviqrhplaekmmdeittvdias | | | |
| | [Salmonella | yrddrlsqvntrtgrcisgntvrlelalls | | | |
| | enterica] | slynlasvewgtcrtnpvemvrkpkisggr | | | |
| | | drrltsqeerrlsryfqeqnpalhaifhla | | | |
| | | ietamrqgeilslrwehidlqhgvahlpmt | | | |
| | | kngssrdvplsrkarhllqgmtvalsgnvf | | | |
| | | hysssgfksawrvalqrlnivdlhfhdlrh | | | |
| | | eaisrlfelgtlnvmevaaisghrslnmlk | | | |
| | | rythlrayqlvskldarrrqtqkiapyfvp | | | |
| | | ypaciesinegsdgccgfrvhlpdfdnlsv | | | |
| | | saasresaleaagvlllrtlakaaqrgerv | | | |
| | | prpgdlpegkhervmihpllsaa | | | |
| WP.070794953 | integrase | msqpsrirkmtlsaaltkyydtvsvhkrgy | 376 | Shufflon | 127 |
| | [Salmonella | qqefwrvsvikrhpvvqkmmdevttvdiaa | | | |
| | enterica] | yrddrlsqesprtgkpisgntvrlelalls | | | |
| | | alynlakvewgtcrtnpvemvrkpkpspgr | | | |
| | | drrltsseerrlsryfqarnaelytifhla | | | |
| | | letgmrqgeilslrwehidlqhgvahlpvt | | | |
| | | kngstrdvplsrrarnllhelpvqlsgavf | | | |
| | | hykstgfksawrvalqslkiedlhfhdlrh | | | |
| | | eaisrlfelgtlnvmevaaisghkslnmlk | | | |
| | | rythlrayqlvskldtrrrqsqkiatyfvp | | | |
| | | ypavleeagdgfrvhlhdfegmsvsgdtpe | | | |
| | | samdaasvvllrtlaiaaqrgervprpgdl | | | |
| | | pvhtgvmidplpgmrq | | | |
| WP_079899823 | site-specific | mlpsvrvkkislfraldryldtvsvhkrgy | 379 | Shufflon | 128 |
| | integrase | qqefwrvsvikrhpvaqkmmdevtsvdias | | | |
| | [Salmonella | yrderlsqvntrtgkpisgntvrlelalms | | | |
| | enterica] | alynlakvewgtcrtnpveivrkpkpssgr | | | |
| | | drrltsseerrlskyfqvrnaelytifhla | | | |
| | | letgmrqgeilslqwehidlqhgvahlpvt | | | |
| | | kngsvrdvplsrrarnllhelpvqlsgtvf | | | |
| | | hykstgfksawrvalqklkienlhfhdlrh | | | |
| | | eaisrlfelgtlnvmevaaisghkslnmlk | | | |
| | | rythlrayqlvskldtrrrqsqkiatyfvp | | | |
| | | ypaileeagdgfrvhlhdfegmsvsgdtre | | | |
| | | samdtasvvllralataaqrgervprpgdl | | | |
| | | plnagvminplagsvpvcv | | | |
| WP_080861315 | site-specific | maqpvrikkmslsaaltkyydtvsvhkrgh | 379 | Shufflon | 129 |
| | integrase | qqefwrvsvikrhpvaqkmmdevttvdiaa | | | |
| | [Citrobacter | yrddrlaqvnprtgkpisgntvrlelalls | | | |
| | braakii] | alynlakvewgtcranpveavrkpkpspgr | | | |
| | | drrltsseerrlsryfqarnaelytifhla | | | |
| | | letsmrqgemlalrwehidlqhgvahlpvt | | | |
| | | kngsprdvplsrrarsllqqlsvqisgpvf | | | |
| | | hykssgfksawraalqrlkienlhfhdlrh | | | |
| | | eaisrlfelgtlnvmevaaisghkslnmlk | | | |
| | | rythlrayqlvskldvrrrqsqkiatyfvp | | | |
| | | ypaemedtadgfrvhlhdfeglsvsghtre | | | |
| | | aamdaasvmllrrlataaqhgervprpgdl | | | |
| | | plhagvminplagaapvfv | | | |
| WPJ187639219 | MULTISPECIES: | mfrkikirkmtlnraldkylktvsihkkgh | 374 | Shufflon | 130 |
| | integrase | lqefyrvnvikrhpiaerymddittvdian | | | |
| | [Enterobacteri | yrdqrlaqinprtgrqitgntvrlelalls | | | |
| | aceae] | slfniarvewgtcrmnpvelvrkpkissgr | | | |
| | | drrltsgeerrlsryfrdknqqlyvifhla | | | |
| | | letamrqgeiltlrwehldlqhgvahlpet | | | |
| | | knglprdvplsrkarnylqilpqqingnvf | | | |
| | | sytssgfksawrtalldlkienlhfhdlrh | | | |
| | | eaisrffelgtlnvievaaisghrslnmlk | | | |
| | | rythlrayqlvskldarrkqtskispyfvp | | | |
| | | ypatvrcrnglfvvtlhdfdletraetrel | | | |
| | | aishasvlllrtlaqaaqrgervptpgelp | | | |
| | | anidervmicpltn | | | |
| AAV47109 | phage integrase/site- | mylkarqdeltestiqsqeyrleafeqfcr | 330 | SNJ2 | 131 |
| | specific recombinase | eegienlndlsgrdlyayrvwrregngkgr | | | |
| | [Haloarcula | deiepitlrgqlatvrsflrfaaevdavpe | | | |
| | marismortui | dlrtkvplptisnagevsastldperadvi | | | |
| | ATCC | ldylqmykyasrvhvialllwhtgarmgai | | | |
| | 43049] | rgldiddceleqdnpgiqfvhrpqtdtplk | | | |
| | | ngekgqrwnaisdhvanvlqdyidgprepv | | | |
| | | fdehgrrplvttpqgraststfrttmyrvt | | | |
| | | rpcwrgaecphdrdpeeceatsnrkastcp | | | |
| | | sarsphdvrsgrvtayrredvprrvvsdrl | | | |
| | | nasdqildkhydrrgerekseqrrdylpev | | | |
| ACV10974 | integrase domain | mrlvemrrwpgvseelsplspeegidrflr | 351 | SNJ2 | 132 |
| | protein SAM domain | hrepsvrestmrnartrlrffrewceerei | | | |
| | protein | enlntltgrdladfvawrrgdvkaltlqkq | | | |
| | [Halorhabdus | lstirtalrfwadveavqeglaeklhapel | | | |
| | utahensis | pdgaesrdvaldadraadileylrelhyas | | | |
| | DSM | rdhvvmeilwrtamrrgalrsidvddlrpd | | | |
| | 12940] | dhaivlrhridegtklkngesgerwvylgp | | | |
| | | styqviddyldnpdrydvtddhgreplltt | | | |
| | | pygrpigdtiyswvnrltqpcriggcphdr | | | |
| | | dpsdpstcdalgsdgspsrcpsarsphgir | | | |
| | | rgsithhlntdvspeivsercdvtldvlye | | | |
| | | hydvrtdqekmavrkrqlsef | | | |
| ACV47094 | integrase family | mpdpdlepispveavemyhdamvdela | 351 | SNJ2 | 133 |
| | protein | estrksnkhrlrafiqfcdeeeienlndl | | | |
| | [Halomicrobium | tgrdlykyriwrregngdgrepikkvtlkg | | | |
| | mukohataei | qlatlrsflkfageidsvkpdlyeqlslpa | | | |
| | DSM | mkggedvsestldperaldileyleksqpg | | | |
| | 12286] | srdhiiiallwetggrtgairgldlqdldl | | | |
| | | dgdhprfsgpavhfvhrpetgtplknqksg | | | |
| | | trwnrisektaafiedyiefhrpdvtddhg | | | |
| | | rdplltseygrvagntyrrtlyrvtrpcwr | | | |
| | | geecphdrdldeceathldhaskcpsarsp | | | |
| | | hdvrsgrvtyyrredvprkivqerlnased | | | |
| | | ildrhydrrsnreqaeqrsdflpdV | | | |
| ADE02447 | XerC/D-like | mselesleparavrmylearqdeladwt | 348 | SNJ2 | 134 |
| | integrase | lkshkyrlrafvewceesgvddlteldgr | | | |
| | [Haloferax | dlyefrvwrregnfgvedgetpeeiapvt | | | |
| | volcanii | lksqlttlraflrfaanihavpedfyervp | | | |
| | DS2] | lpklsgtddvsdstlepdratdileylhry | | | |
| | | hyasrrhvefallwetgarmgairgldlrd | | | |
| | | ldldgrtpvvrykhrpdqgtpikngekge | | | |
| | | rfnsvsdrvgtmlqayidgprvdktdef | | | |
| | | grkpllttshgrvsastirqdvyvvtrpcw | | | |
| | | lnqgcphnrdietceavelnhvstcpssr | | | |
| | | sphdvrkgvvtlyrreevprrvvsdrlda | | | |
| | | sdlvldkhydrrgereraeqrrnhlpw | | | |
| AF055992 | Phage | mvigmsddlepigpeqavemyiegrrdels | 349 | SNJ2 | 135 |
| | integrase/site- | dqtlpshvyrleaftqwcaeegienlneit | | | |
| | specific | grnlyayrvwrregngegreevttitlrgq | | | |
| | recombinase | latlraflrfcadidavpedlfskvplptv | | | |
| | [Natrinema | sasegvsdttlepdraveildylqryeyas | | | |
| | sp. J7-2] | rkhitllllwhtgaraggvrgldlrdcele | | | |
| | | gespglqfvhrpetdtplkkgekgerwnsi | | | |
| | | sghvagvlqdyvdgprdnvtddhgrspllt | | | |
| | | trsgrpcistirdtmygltrpcwrgaecph | | | |
| | | drdpeeceatyyakastcpssrsphdvrsg | | | |
| | | rvtayrredvprrvvgdrldasddildrhy | | | |
| | | drmarekaeqrrdylpdl | | | |
| AGB16629 | integrase | mseleplsplealelwlerlqstrseatie | 362 | SNJ2 | 136 |
| | [Halostagnicola | syryrmqsfvewcdeeeidnlndltsrdvf | | | |
| | larsenii | rydserrseglspatlktqlgtlklflefc | | | |
| | XH-48] | drleavpeglyekvevptvelaervndelv | | | |
| | | raeraeqiledlelydrasrrhaifaiawh | | | |
| | | cgcrlgglraldledcffepsdldrlrhqd | | | |
| | | didhealeevdlpflyfrhrpetdtplknk | | | |
| | | kqgerpvalsddvasliksyiqvkrakrsd | | | |
| | | gdrrplfttekgdnarvskssirrdiyilt | | | |
| | | qpcrygtcphnrdeencealkhghearcps | | | |
| | | srsphpirtgaithmrdegwppevvaervn | | | |
| | | atpevirahydhpdpirrmqsrrsflnkea | | | |
| | | dt | | | |
| AHG00321 | integrase domain | msedlqplppkegvdrflehrapsiressm | 337 | SNJ2 | 137 |
| | protein SAM domain | qnarhrlsvflewcdendvddlndltgrdl | | | |
| | protein | safvawrqgdvaaitlqkqlssvrmalrww | | | |
| | [Halorhabdus | adiegveeglaeklhspdlpdgaeskdvfl | | | |
| | utahensis | eadrakralryydrhhyasrdhallaliwr | | | |
| | DSM | tgmrrgavrgldvddldsddqairvehrpd | | | |
| | 12940] | tgtplkngdggnrwvylgprwftiledfva | | | |
| | | npdrknvrdehgrrplfttqqetrptghsi | | | |
| | | ykwviralhpckyaecphdrkpsecealgs | | | |
| | | ssvpskcpsarsphsirr | | | |
| | | gaitnhlneetapetvsermdvsldvlyqh | | | |
| | | ydarterekmavrrhnlpe | | | |
| CAI49276 | XerC/D-like | msrnrsreapsewsprnaaeryikhrasdt | 362 | SNJ2 | 138 |
| | integrase | tessrsgwwyrlklfvewceevgletvsdi | | | |
| | [Natronomonas | qpldideyhdiraeavapvtlegematlqe | | | |
| | pharaonis | ylrylegldavaddlseavhvpnldasqrs | | | |
| | DSM | ndvklstpeamamlqyfretpavrasrkhv | | | |
| | 2160] | flelvwftgarqsglraldlrdvhlddafv | | | |
| | | wfkhrpsegtglknnldgerpvslpsgvvd | | | |
| | | vlreyihenrnsetdvhgraplfttlqgrp | | | |
| | | sgdsvrkwcylatlpclhsdcphgkdresc | | | |
| | | dwtgykyaskcpstrsphrirtgsityqln | | | |
| | | igfptevvanrvnaspktirdhydkadrqe | | | |
| | | rrrrqrrrmesdrrgyvqqmdfdyendigs | | | |
| | | dd | | | |
| CAI50775 | XerC/D-like | msddlepiapaeavemyiearqddctenti | 349 | SNJ2 | 139 |
| | integrase | egqyyrlqaflawcdeeditnlneldgrdl | | | |
| | [Natronomonas | yayrvwrreggysdtelagatlrgdlatlr | | | |
| | pharaonis | aflrfcgeveavppeftdrvplpsvsggad | | | |
| | DSM | vsastldpdraqaileylqqfeyaskrhvi | | | |
| | 2160] | vlllwhagcrvgalraldvddldlagdipn | | | |
| | | atgpgikfvhrpdegtplknkrkserwnti | | | |
| | | segvanviedyiasrrteaeddygrrplis | | | |
| | | trygrmsrsairqelyrvtrpcwyndgcph | | | |
| | | drdpdeceatddgsmskcpssrsphdvrsg | | | |
| | | rltfyrlrevdekvvsdrmdaseeildkhy | | | |
| | | drrserqkaeqrrshlpdv | | | |
| ELZ11643 | phage | mgddlepiapeqalemyvegrrdelsdqtl | 345 | SNJ2 | 140 |
| | integrase/site- | pshvyrleaftqwceeegienlntltgrdl | | | |
| | specific | yayrvwrregngdgrdevatvtlrgqlatl | | | |
| | recombinase | raflqfcadidavpeelyskvplpsvsase | | | |
| | [Haloterrigena | gvsdttldperaveildylqryeyasnhvt | | | |
| | thermotolerans | vlllwhtgaraggiraldlrdcelegespg | | | |
| | DSM | vqfvhrpetdtrlkkgekgerwnsisghva | | | |
| | 11522] | gvlldyvegprkdvtddhgrspllttrsgr | | | |
| | | psvstirntmygvtrpcwrgaecphdrdpe | | | |
| | | dcdatyyakastcpssrsphdvrsgrvtay | | | |
| | | rredvprrvvgdrldasddildrhydimar | | | |
| | | ekaeqrrdylpdl | | | |
| WP_004515348 | phage | mylkarqdeltestiqsqeyrleafeqfcs | 330 | SNJ2 | 141 |
| | integrase/site- | eegienlndlsgrdlyayrvwrregngker | | | |
| | specific | egiepitlrgqlatvrsflrfaaevdavpe | | | |
| | recombinase | nlrtkvplptingagevsastldperadvi | | | |
| | [Haloarcula | ldylqmykyasrthvivlllwhtgarmgai | | | |
| | vallis mortis] | rgldiddcelegsdpgiefvhrpqsdtpik | | | |
| | | ngekgqrwnaisehvanvvqdyingpresv | | | |
| | | fdehgrrplittqqgraststyrmainyrv | | | |
| | | trpcvvrgaecphdrdpeeceatsnkkast | | | |
| | | cpsarsphdvrsgrvtayrredvprrvvsd | | | |
| | | rldasdqildkhydrrgerekseqrrdylp | | | |
| | | ev | | | |
| NP_039778 | ORF D-335 | mtkdktrykygdyilrerkgryyvykleye | 335 | SSV | 142 |
| | [Sulfolobus | ngevkeryvgpladvvesylkmklgvvgdt | | | |
| | spindle- | plqadppgfepgtsgsgggkegterrkial | | | |
| | shaped virus 1] | vanlrqyatdgnikafydylmnergisekt | | | |
| | | akdyinaiskpyketrdaqkayrlfarfla | | | |
| | | srniihdefadkilkavkvkkanadiyipt | | | |
| | | leeikrtlqlakdysenvyfiyrialesgv | | | |
| | | rlseilkvlkeperdicgndvcyyplswtr | | | |
| | | gykgvfyvfhitplkrvevtkwaiadferr | | | |
| | | hkdaiaikyfrkfvaskmaelsvpldiidf | | | |
| | | iqgrkptrvltqhyvslfgiakeqykkyae | | | |
| | | wlkgv | | | |
| NP_944456 | integrase | mpnfyvgskfyvkeikgkyyvysiengddg | 328 | SSV | 143 |
| | [Sulfolobus | kqrhtyigsleqivneyydmkcgrrdlnpg | | | |
| | spindle-shaped | spaweagirgtppktpdanddelkgvriid | | | |
| | Virus 2] | snltssnnseisasdllkfeftlrqkkitd | | | |
| | | ktikeyincvkqgrkesnncikawrnfykl | | | |
| | | vlnrdppeslkikrtkpdlrvptleevrkt | | | |
| | | lstvkeypnlylfyrlllesgsresealkv | | | |
| | | lndynpqneireegfsiyilnwtrgqkksf | | | |
| | | yifhvtelkqikiskayvdkyvrrlnlvpp | | | |
| | | kyirkffatkalelgipsevvdflegrtpg | | | |
| | | diltkhyldlltlakkyyplyaewlytf | | | |
| NP_963933 | ORF D355 | meflsssfsltgdkiiiilfkclrdkykwa | 355 | SSV | 144 |
| | [Sulfolobus | egmgnkvftfgdirirevkgkyyvyliekd | | | |
| | virus | negnrrdhyvgsldqivkdyisikvrgtgf | | | |
| | Ragged Hills] | epaqafasgasvrpmgdtpippdlknkgvi | | | |
| | | tkdmeitrdklneffewcvkkrknsidtck | | | |
| | | dyilylkrplnknkkwsvfayrlyyeflgk | | | |
| | | edkakelkvekkmsipvyripsleeikkvl | | | |
| | | nhederirilyrlllesgirlkealfilnn | | | |
| | | ydpaldqmedgfyvytvnlirkskksfyaf | | | |
| | | hitplqktyitesiidhtdlpvkpkfirkf | | | |
| | | vatkmlelgipsevvdffqgrtpssilskh | | | |
| | | yldlltlakkeykkyaewltkyvll | | | |
| NP_963973 | ORF 1-340 | mpsfyvgsnfyikeikgkyyvysiekgedn | 340 | SSV | 145 |
| | [Sulfolobus | kqrhhyiapldkviefyisngglrgyppng | | | |
| | virus | gvgvpptmgacrapdpgsnpgrgaflyvds | | | |
| | Kamchatka 1] | nnelkgvriidsnltssnnseisasdllkf | | | |
| | | eltlrqkniseetikkyiscvkqgrkesnn | | | |
| | | cikawrnfyrlvlnrdppselkpkktkpdl | | | |
| | | kvptleevretldkvkqypslyllyrllle | | | |
| | | sgsrlrealkllnnynpqneirgdgfsiyv | | | |
| | | lnwtrgqkksfylfhitelkaekvtegqit | | | |
| | | savrrlnlvppkyirkfvatklfelgvsse | | | |
| | | vvdflegrtpgniltkhyldlltlakkeyk | | | |
| | | kyaewlkqii | | | |
| YP_003331413 | Integrase | matiilgdkmakdktrykygdiilrerkgr | 347 | SSV | 146 |
| | [Acidianus | yyiykletingetketyvgplidvvesylk | | | |
| | spindle-shaped | mkeigvlgvspnvagppgfepgtyglkarr | | | |
| | Virus 1] | eldelrdraeelkevailrkyvtegnleef | | | |
| | | yswatmkkgidertaklyvrqiqkpfekkr | | | |
| | | nrifayrafarfliekgigvsdileklkti | | | |
| | | sskpdlrvptldevrktlqlakeysenvyf | | | |
| | | vyrlalesgsrlseilkvlkepekdvcdnd | | | |
| | | icyyplawtrgqksvfyvfhltplrkidit | | | |
| | | qwaisdferrndeaipikyirkfvatelag | | | |
| | | lginfdiidfiqgrkpsrvltqhyvsmfai | | | |
| | | akenykkyaewirqtlt | | | |
| YP 003331458 | integrase | mivislfkhqrdnykwaegmgnkvftfgdi | 334 | SSV | 147 |
| | [Sulfolobus | rirevkgkyyvyliekdnegnrrdnyvgkk | | | |
| | spindle- | levvifyiknaktgvvgafppqgsgpwdqg | | | |
| | shaped virus | snpcpatflsplsnnelnvvitneasftgd | | | |
| | 6] | kkteklpsemelfafyndcvkkvsretcke | | | |
| | | yvnylrkpldvnnkasilawkkyykwkgdl | | | |
| | | eawkkiktkksgvdlrvpseaeikewltkv | | | |
| | | kgtkvellfklllesgirlteavklvneyd | | | |
| | | pknetiessyyiytmnwsrgskrvfyvfhv | | | |
| | | tplqklqitynyakklfhelkidpkyvrkf | | | |
| | | vatkclelnipaevvdflegrtptqiltrh | | | |
| | | yldlltltkkyyplyaewlrqtlt | | | |
| YP_003331490 | integrase | mpnfyvgskfyvkeikgkyyvysiengddg | 336 | SSV | 148 |
| | [Sulfolobus | kqrhtyigsleqiitsylelgvwgvppqcg | | | |
| | spindle-shaped | rrdlnpgspaweagirgappktptdnnvel | | | |
| | virus | kgvriidsnltssnnseisvsdlikfefal | | | |
| | 7] | rqkkitdktikeylscikrnkkdsnncika | | | |
| | | wrnfyrlvlnrdppeslkikrtkpdlrvpt | | | |
| | | leevrktlstvkeypnlylfyrlllesgsr | | | |
| | | esealkvlseynsqnemqevgfsiyilnwt | | | |
| | | rgqkksfylfhvtelkqikiskayvdkyvk | | | |
| | | klnltppkyirkftatkmlelgipsevvdf | | | |
| | | iqgrtpsevltkhyldlltlakkeykkyae | | | |
| | | wlrqni | | | |
| YP_00767X011 | integrase | madkprtvtlgefrlrylknkvyvykvkng | 323 | SSV | 149 |
| | [Sulfolobales | yeeeyiaplerlvehflstadakgqdrkdg | | | |
| | Mexican | kgqidvlqsapenvgetkvnrnevtvssvi | | | |
| | fusellovirus | elqrffnwcvkfaseqtcntyvkylqrppn | | | |
| | 1] | sthpsiravvrayykwkgkedklkelklpr | | | |
| | | sgsdlrlvtedevkralknssgdevahyil | | | |
| | | sllvesglrlsevvkvlneyepsqdtaynt | | | |
| | | fnvynvnwrrgrkntlymfhisplrqmtld | | | |
| | | yentrvklaryidakfmrkfvatkmfelei | | | |
| | | paevidfiqgrapttvatkhyiylftiark | | | |
| | | yyeekwvpyvrallnlnsqgeskt | | | |
| YP_009177672 | hypothetical protein | mwgepllygagdstvtlvpkplyvyvhtvk | 399 | SSV | 150 |
| | [Aeropyrum pernix | skgriyqylvveeylgqgrrrtilrmrlee | | | |
| | ovoid virus 1] | avrkllnnekkdsaetagwcggwdlnprrp | | | |
| | | tptglkpapskpfssmviekrdsgdgesep | | | |
| | | stkqdgglivsetlasrflewldlpedsrq | | | |
| | | lrdyrnnlrlligkpldcatlhefasqskr | | | |
| | | kyetasrllsfvaskrglglrqlaaelrec | | | |
| | | lgkkprsgsdtyvppdssileaarrlegtr | | | |
| | | vyhvflllvgsgarlstvhwllrqgldssr | | | |
| | | lvcledrgfcryhvdyvkgeklqwalyspr | | | |
| | | efwervleeprltlsynrvqeqiagagvka | | | |
| | | khirnwvynkmlslgmpegvvefivghkas | | | |
| | | sigrrhymnmivqadmwyttylpvipkslk | | | |
| | | lscttcyeg | | | |
| WP_009990677 | recombinase XerD | mkldlgsppesgdlynafmaliiagagngt | 291 | XerA | 151 |
| | [Saccharolobus | iklystavrdfldfinkdprkvtsedlnrw | | (Crenar | |
| | solfataricus] | issllnregkvkgdevekkraksvtiryyi | | chaeota) | |
| | | iavrrflkwinvsvrppipkvrrkevkald | | | |
| | | eiqiqkvlnackrtkdkliirllldtglra | | | |
| | | nellsvlvkdidlennmirvrntkngeeri | | | |
| | | vfftdetklllrkyikgkkaedklfdlkyd | | | |
| | | tlyrklkrlgkkvgidlrphilrhtfatls | | | |
| | | lkrginvitlqkllghkdikttqiythlvl | | | |
| | | ddlrneylkamsssssktpp | | | |
| WP_012021561 | recombinase XerD | mklqlgepptdadpfiyfmeslkfsgagqg | 286 | XerA | 152 |
| | [Metallosphaera | tiklystaiqdflqfvkkdprsvttqdvid | | (Crenar | |
| | sedula] | wigslnsrkgrsrvvdkrgrsatirsyvia | | chaeota) | |
| | | vrrflkwlgvnvkppvprirspermalree | | | |
| | | divallsacrrlrdkvivsllvdtglrsse | | | |
| | | llslrrsdvdlermlirvretkngeerivf | | | |
| | | ftsrtatllrqylrktqdkesddaplfnls | | | |
| | | yqalyklikrlgrktgltwlrphvlrhtfa | | | |
| | | tnairrgvplpavqrlmghkdikttqiyth | | | |
| | | lvtedlenayrrafet | | | |
| WP_010901720 | integrase | mpaetneylsrfveymtgerksrytikeyr | 283 | XerA | 153 |
| | [Thermoplasma | flvdqtlsfmnkkpdeitpmdieryknfla | | (Euryar | |
| | acidophilum] | vkkrysktsqylaikavklfykaldlrvpi | | chaeota) | |
| | | nltppkrpshmpvylsedeakrlieaassd | | | |
| | | trmyaivsvlaytgvrvgelcnlkisdvdl | | | |
| | | qesiinvrsgkgdkdrivimaeecvkalgs | | | |
| | | yldlrlsmdtdndylfvsnrrvrfdtstie | | | |
| | | rmirdlgkkagiqkkvtphvlrhtfatsvl | | | |
| | | rnggdirfiqqilghasvattqiythlnds | | | |
| | | alremytqhrpry | | | |
| WP_011013007 | recombinase XerC | mrektlrsevleefatylelegkskntirm | 286 | XerA | 154 |
| | [Pyrococcus | ytyflskfleegysptardalrflaklrak | | (Euryar | |
| | furiosus] | gysirsinlvvqalkayfkfeglneeaerl | | chaeota) | |
| | | rnpkipktlpkslteeevkklievipkdki | | | |
| | | rdrlivlllygtglrvselcnlkiedinfe | | | |
| | | kgfltvrggkggkdrtipipqpllteikny | | | |
| | | lrrrtddspylfvesrrknkeklspktvwr | | | |
| | | ilkeygrkagikvtphqlrhsfathmlerg | | | |
| | | idiriiqellghaslsttqiytrvtakhlk | | | |
| | | eaveranllenligge | | | |
| WP_011249728 | recombinase XerC | msepnevieefetyldlegksphtirmyty | 282 | XerA | 155 |
| | [Thermococcus | yvrrylewggdlnahsalrflahlrkngys | | (Euryar | |
| | kodakarensis] | nrslnlvvqalrayfrfeglddeaerlkpp | | chaeota) | |
| | | kvprslpkaltreevkrllsvipptrkrdr | | | |
| | | livlllygaglrvselcnlkkddvdldrgl | | | |
| | | ivvrggkgakdrvvpipkyladeirayles | | | |
| | | rsdeseyllvedrrrrkdklstrnvwyllk | | | |
| | | rygqkagvevtphklrhsfathlleegvdi | | | |
| | | raiqellghsnlsttqiytkvtvehlrkaq | | | |
| | | ekaklieklmge | | | |
| WP_012034516 | integrase | mcmgigmdyvavfidekrlssspgtirqyg | 278 | XerA | 156 |
| | [Methanocella | milnrfykytgkqpemvvrpeivrylnylm | | (Euryar | |
| | arvoryzae] | fekhlskttvanvlsvlksfysfmldngyv | | chaeota) | |
| | | ssnptrginnikldkkapvyltvsemndll | | | |
| | | dtaidtrdriivrllyatgvrvselvnirk | | | |
| | | kdidfdrctikvfgkgakerivlvpetvvk | | | |
| | | emydyaaslsnddrlfnltprtvqrdikql | | | |
| | | arrakinknvtphklrhsfathmlqnggnv | | | |
| | | vaiqkllghsslnttqiythynvdelkemy | | | |
| | | grthplgk | | | |
| WP_012997197 | integrase | msdkfmdyvdyelekfkeylrgekrsenti | 284 | XerA | 157 |
| | [Aciduliprofundum | keyahfisdmlryfhkraeditpgdlnkyk | | (Euryar | |
| | Boonei] | mylstkrkysknslylatkairsyfkyknl | | chaeota) | |
| | | dtaknlsspkrprqmpkylsedevkrliea | | | |
| | | ssenprdyaiisllaysglrvselcnlkie | | | |
| | | dvdfnerivyvhsgkgdkdrivvvsprvie | | | |
| | | alqnylytreddmeylfasqksnkisrvqv | | | |
| | | frivkkyaekagikkevtphvlrhtlattl | | | |
| | | lrrgvdirfiqqflghssvattqiythvdd | | | |
| | | allksvydkvlqey | | | |
| WP_042690709 | recombinase XerC | mdevieefetyldlegkspntirmysyyvr | 278 | XerA | 158 |
| | [Thermococcus | rylewggalnarsalrflarlrregysnrs | | (Euryar | |
| | nautili] | lnlvvqalrayfrfeghdeeaeklkppkvp | | chaeota) | |
| | | rslpkaltreevkrllsvipptrkrdrliv | | | |
| | | lllygaglrvselvnlkksevdlergiivv | | | |
| | | rggkgakdrvvpipeflveeirsyletrsd | | | |
| | | sseyllveerrknkdrlstktvwyllkkyg | | | |
| | | kragvevtphrlrhsfathmlergvdirai | | | |
| | | qellghsnlsttqiytkvtvehlrkaqeka | | | |
| | | rlmeglve | | | |
| NP_232049 | site-specific | msealspdqglveqfldtmwferglaentv | 302 | XerCD | 159 |
| | tyrosine | asyrndlskllewmaqnqyrklfisfaglq | | | |
| | recombinase | eyqswlseqnykptskarmlsairrlfqyl | | | |
| | XerD | hrekvraddpsallvspklptrlpkdlsea | | | |
| | [Vibrio cholerae | qveallsapdpqsplelrdkamlellyatg | | | |
| | O1 biovar EI Tor | lrvtelvsltmenmslrqgvvrvmgkggke | | | |
| | str. | rlvpmgenaievvietflqqgrslllgeqt | | | |
| | N16961] | sdivfpssrgqqmtrqtfwhrikhyaviag | | | |
| | | idveklsphvlrhafathllnygadlrvvq | | | |
| | | mllghsdlsttqiythvaterlkqlhnehh | | | |
| | | pra | | | |
| NP_417370 | site-specific | mkqdlarieqfldalwleknlaentlnayr | 298 | XerCD | 160 |
| | recombinase | rdlsmmvewlhhrgltlataqsddlqalla | | | |
| | [Escherichia coli | erleggykatssarllsavrrlfqylyrek | | | |
| | str. | freddpsahlaspklpqrlpkdlseaqver | | | |
| | K-12 substr. | llqaplidqplelrdkamlevlyatglrvs | | | |
| | MG 1655] | elvgltmsdislrqgvvrvigkgnkerlvp | | | |
| | | lgeeavywletylehgrpwllngvsidvlf | | | |
| | | psqraqqmtrqtfwhrikhyavlagidsek | | | |
| | | lsphvlrhafathllnhgadlrvvqmllgh | | | |
| | | sdlsttqiythvaterlrqlhqqhhpra | | | |
| NP_418256 | site-specific | mtdlhtdverylrylsverqlspitllnyq | 298 | XerCD | 161 |
| | tyrosine | rqleaiinfasenglqswqqcdvtmvrnfa | | | |
| | recombinase | vrsrrkglgaaslalrlsalrsftdwlvsq | | | |
| | [Escherichia coli | nelkanpakgvsapkaprhlpknidvddmn | | | |
| | str. | rlklidindplavrdramlevmygaglrls | | | |
| | K-12 substr. | elvgldikhldlesgevwvmgkgskeirlp | | | |
| | MG 1655] | igrnavawiehwldlrdlfgseddalflsk | | | |
| | | lgkrisarnvqkrfaewgikqglnnhvhph | | | |
| | | klrhsfathmlessgdlrgvqellghanls | | | |
| | | ttqiythldfqhlasvydaahprakrgk | | | |
| WP_006927519 | tyrosine recombinase | mdkhirdflrylflerryarntirsygtdl | 306 | XerCD | 162 |
| | XerC [Caldithrix | lqfeefleqhftutnipwslvdkrvirffl | | | |
| | abyssi] | irlqeqkiskrsiarklatlksffiyllkn | | | |
| | | giiesnpvatvkmpklekklpehlgpaeie | | | |
| | | allrlpklntfeglrdlailelfygtgirl | | | |
| | | selinlkvsqvdfqenlirvigkgnkeriv | | | |
| | | pfggsaklilekylsirpqfaensvdnlfv | | | |
| | | lksgkkmypmavqrivkkyltqasnlkqks | | | |
| | | phvlrhtyathllnqgadirvvkdllghen | | | |
| | | lattqiythlsiehlkkvynqahpratnks | | | |
| | | sknrrr | | | |
| WP_011848048 | tyrosine recombinase | mstqtaevsalntqwlqtferylsterqls | 306 | XerCD | 163 |
| | XerC [Shewanella | ahtvrnylyelnrgsdllpdgvnllnvsre | | | |
| | baltica] | hwqqvlaklhrkglsprslslclsavkqwg | | | |
| | | efilregvielnpakglsapkqakplpkni | | | |
| | | dvdaishlldiegtdplslrdkammelfys | | | |
| | | sglrlaelaalnlssvqydlkevrvlgkgn | | | |
| | | kerivpvgrlaiaallnwlncrkqipcedn | | | |
| | | alfvtekgkrlshrsiqarmakwgqeqals | | | |
| | | vrvhphklrhsfathmleasadlravqell | | | |
| | | ghanlattqiytsldfqhlakvydnahpra | | | |
| | | kktqdk | | | |
| WP_012175913 | tyrosine recombinase | mskdhgaypakpladafveslasekgyspn | 308 | XerCD | 164 |
| | XerC [Desulfococcus | tcraysadlkeflaflsppddtehpvcldd | | | |
| | oleovorans] | isviairgylaflhkkkmdkstvsrklsvl | | | |
| | | rsffrylekrgimtgnparavlspkigrki | | | |
| | | paflsvddmfrlldastgdtlldlrnraif | | | |
| | | etiystgirvseaagldaahvetdervfrv | | | |
| | | ygkgakervvpvgkkalasiaayrtrlfee | | | |
| | | tgigveegplflnknrgrlttrsmdrilkq | | | |
| | | talrcgltvslsphalrhsfathmldagad | | | |
| | | lrtvqeilghkslsttqkythvsmdklmev | | | |
| | | ydhahprk | | | |
| WP_031544907 | site-specific | mnfkryieeyllflsvekglsqssissyrq | 296 | XerCD | 165 |
| | tyrosine | dlmqyeaflsdhsaldpsqidtellirflk | | | |
| | recombinase XerD | elrhagksaktisrmqstlknfhqflvndg | | | |
| | [Salinicoccus | itthnpalrlhsikeakklpvyltveemek | | | |
| | luteus] | llstpdqsvagvrdksmmellyasglrvse | | | |
| | | lidirtsdlntdmgyirimgkgskerivpi | | | |
| | | tdfvgelleqymsnermallkddvveelfi | | | |
| | | tnrgrgftrqglwktikkyelasgigknit | | | |
| | | phtfrhsfathlvengadlravqemlghsd | | | |
| | | isttqiytqisavkiremykkfhprk | | | |
| WP_041330811 | tyrosine recombinase | mqenfnkyleyltveknvsvytlrnyrtdl | 307 | XerCD | 166 |
| | XerC | igfinyliekkvsstdrvdryilrdymssl | | | |
| | [Dehalococcoides | iekgivkgsiarklsavrsfyrylmregli | | | |
| | mccartyi] | qknptlnassprldkrlpefittaevskll | | | |
| | | ripdsstpqglrdkafmellyasglrvsel | | | |
| | | vkldienldlhshqirvwgkgskerivlmg | | | |
| | | lpaiqsiqtylnlgrpllkskrntpalfln | | | |
| | | pnggrlsarsfqerldklahqagiekhvhp | | | |
| | | hmlrhtfathlldggadlrvvqellghsnl | | | |
| | | sttqiythvtksqarkvymsshplakpqnd | | | |
| | | isgsede | | | |
| WP_044141062 | site-specific | mndqlsdfihfmtverglsentivsykrdl | 296 | XerCD | 167 |
| | tyrosine | qnylsflmtheqltdikdvtrlhiihylkq | | | |
| | recombinase XerD | lkeegkssktsvrhlssirsfhqfllrekv | | | |
| | [Bacillus pumilus] | ttddpswnietqkterklpkvlsleevekl | | | |
| | | ldtpnqhtpfdyrdkamlellyatgirvse | | | |
| | | mldltladvhltmgfircfgkgrkerivpi | | | |
| | | geacasaieeylekgrskllkkqpadalfl | | | |
| | | nhhgkkmsrqgfwknlkkraleagiqkelt | | | |
| | | phtlrhsfathllengadlravqemlghad | | | |
| | | isttqiythvtktrlkdvyhkfhpra | | | |
| WP_047052972 | tyrosine recombinase | mshsplfacvdrflrylgverqlspitltn | 300 | XerCD | 168 |
| | XerC [Klebsiella | yqrqlealialaddaglkswqqcdaaqvrs | | | |
| | aerogenes] | favrsrraglgpaslalrlsalrsffdwmv | | | |
| | | sqgelaanpakgiaapkiprhlpknidvdd | | | |
| | | vnrlldidlndplavrdramlevmygaglr | | | |
| | | lselvnldiqhldlesgevwvmgkgskerr | | | |
| | | lpigrnavawiehwldlrglfggdddalfl | | | |
| | | sklgkrisarnvqkrfaewgikqglnshvh | | | |
| | | phklrhsfathmlessgdlrgvqellghan | | | |
| | | lsttqiythldfqhlasvydaahprakrgk | | | |
| WP_053463963 | site-specific | metnydvvieeylkfiqiekglsantigay | 299 | XerCD | 169 |
| | tyrosine | rrdlnkykeylvlkkinnidfidreiiqqc | | | |
| | recombinase XerD | lgylhddghsaksiarfistvrsfhqfalr | | | |
| | [Staphylococcus | eryaakdptvlietpkyerrlpdvldvedv | | | |
| | camosus] | lalletpdlsknngyrdrtilellyatgmr | | | |
| | | vtelihvrvedvnlimgfvrvfgkgskeri | | | |
| | | iplgetvidylkkyietvrpqllkqavtdv | | | |
| | | lflnlhgkplsrqgiwklikqygvkanikk | | | |
| | | kltphslrhsfathllengadlravqemlg | | | |
| | | hsdisttqlythvsksqirkmynefhpra | | | |
| WP_057085168 | tyrosine recombinase | mnpdsplsapaeaflrylrverqlspltqs | 302 | XerCD | 170 |
| | XerC [Dickeya | syahqlqviidmlsasgitdwqaldaagvr | | | |
| | solani] | avvarskrdglnaaslaqrlsalrsfldwl | | | |
| | | vgrgelkanpargvpapkagrhlpknmdvd | | | |
| | | emsrlldidlsdplavrdramlevmygagl | | | |
| | | rlaelvgldcghvdldsgevwvmgkgsker | | | |
| | | klpigatavtwlrhwlairdiyapeddaif | | | |
| | | isslgkrismrnvqkrfaewgvkqgvnshv | | | |
| | | hphklrhsfathmlessgdlravqellgha | | | |
| | | nlsttqiythldfqhlasvydaahprarrg | | | |
| | | kp | | | |
| WP_066352736 | tyrosine recombinase | meyevvdsflnyikaaknqsentlkayand | 304 | XerCD | 171 |
| | XerC [Fervidicola | lgqfieyleqnkmsetkslknithldirgf | | | |
| | ferrireducens] | laylkekgvakksitrklsalrsffkyltt | | | |
| | | egiisedptkmvqgmklpkklplfiypaei | | | |
| | | eallsapkndvlgirdraimellyatgvrv | | | |
| | | gelvsiklkdvnmganfiivygkgsrermv | | | |
| | | ffgskaaesleeylkksrpylvknlsceyl | | | |
| | | finkngtrltdrsvrriidkyvkelslnkn | | | |
| | | isphtlrhtfathmlnngadlktvqellgh | | | |
| | | vslsttqlythvtkerlkeiydkvfprakk | | | |
| | | kees | | | |
| WP_074824603 | tyrosine recombinase | msertepltcpslqqpvdnflrylrverql | 308 | XerCD | 172 |
| | XerC [Pragia | spytlksyqrqlaalidllvnigltdwtkl | | | |
| | fontium] | daagvrmlvtrskrsglesaslalrlsalr | | | |
| | | sfldwlvgqgiiganpakgistprkgrhlp | | | |
| | | knmdvdevnhlldidlndplavrdrtmlel | | | |
| | | mygaglrlseligldcrqvnldageirvvg | | | |
| | | kgskerklpigrmavtwlnrwlpmrefyap | | | |
| | | dddalfvskhgnrisarnvekrfaewgvkq | | | |
| | | gisshvhphklrhsfathmlessgdlravq | | | |
| | | ellghanltttqiythldfqhltkvydaah | | | |
| | | prakrgkp | | | |
| WP_082736062 | tyrosine recombinase | mllfqyieaflnhmrveksasnftlssykt | 303 | XerCD | 173 |
| | XerC | dlsqffaflsqkkginpeevgvelinhnsv | | | |
| | [Syntrophomonas | rkylaqmqekglsratmarklaalrsfikf | | | |
| | wolfei] | lcreniladnpitavstpkqerklprflyt | | | |
| | | remellmnapdlsmaagkrdrailetlyas | | | |
| | | glrvseltnldkpdidfgedyikvlgkggk | | | |
| | | erivplgskarealllylqqgrvyleakgq | | | |
| | | aspalflnkngqrlstrsirniinkyveti | | | |
| | | ainqkvsphtlrhsfathllnngadlrsvq | | | |
| | | ellghvklsttqiythlsrekikdihqqth | | | |
| | | prr | | | |
| WP_083945456 | tyrosine recombinase | rnniimcdnkqtnqidkfidqfmfylrvek | 317 | XerCD | 174 |
| | XerC [Sporomusa | nssrhtllnyqrdiyqfvefvsnqgggerp | | | |
| | sphaeroides] | fsyvtplllrsylahlksqeyakatimrri | | | |
| | | aalrsffrflcrenilsenpcdavrtpkle | | | |
| | | kklpvfldanevselmalpddsplgfrdka | | | |
| | | vlellyatgvrvnelagitlpdidvegrti | | | |
| | | ivsgkgakerivlmgktaaaflekylqrar | | | |
| | | pvlctktgeygrqtkkqhsylfvnnrggpl | | | |
| | | tdrsirrivekyveemalkknvsphtlrht | | | |
| | | fathlldngadlrtvqellghvnlsttqly | | | |
| | | thitterlkanykkshpra | | | |
| WP_000682431 | integrase | mkhpleelkdptenlllwigrflrykctsl | 362 | XerH | 175 |
| | [Helicobacter pylori] | snsqvkdqnkvfeclnelnqacsssqlekv | | | |
| | | ckkarnagllgintyalpllkfheyfskar | | | |
| | | literlafnslknidevmlaeflsvytggl | | | |
| | | slatkknyriallglfsyidkqnqdeneks | | | |
| | | yiynitlknisgvnqsagnklpthlnneel | | | |
| | | ekflesidkiemsakvrarnrllikiivft | | | |
| | | gmrsnealqlkikdftlengcytilikgkg | | | |
| | | dkyravmlkafhiesllkewlierelypvk | | | |
| | | ndllfcnqkgsaltqaylykqveriinfag | | | |
| | | lrrekngahmlrhsfatllyqkrhdlilvq | | | |
| | | ealghaslntsriythfdkqrleeaasiwe | | | |
| | | en | | | |
| NP_418732 | (FimB) regulator for | | 0 | Fim | — |
| | fimA [Escherichia | | | | |
| | coli | | | | |
| | str. K-12 substr. | | | | |
| | MG 1655] | | | | |
| NP_418733 | (FimE) regulator for | | 0 | Fim | — |
| | fimA [Escherichia | | | | |
| | coli | | | | |
| | str. | | | | |
| | K-12 substr. | | | | |
| | MG 1655] | | | | |
| WP_001295805 | (HbiF) | | 0 | Fim | — |
| | MULTISPECIES: | | | | |
| | DNA recombinase | | | | |
| | [Enterobacteriaceae] | | | | |
| SPY37376 | (mrp1) fimbriae | | 0 | Fim | — |
| | recombinase [Proteus | | | | |
| | mirabilis] | | | | |
| WP_010891107 | (PcL1) hypothetical | | 0 | Fim | — |
| | protein [Chlorobium | | | | |
| | limicola] | | | | |
| AF112374 | | | 0 | DIRS- | — |
| | | | | like | |
| AF442732 | | | 0 | DIRS- | — |
| | | | | like | |
| AYCK01014057 | | | 0 | DIRS- | — |
| | | | | like | |
| CAKA01505858 | | | 0 | DIRS- | — |
| | | | | like | |
| AFNY01032878 | | | 0 | DIRS- | — |
| | | | | like | |
| AANH01008719 | | | 0 | DIRS- | — |
| | | | | like | |
| AERX01068420 | | | 0 | DIRS- | — |
| | | | | like | |
| AGAJ0104998 | | | 0 | DIRS- | — |
| | | | | like | |
| GBDH01091653 | | | 0 | DIRS- | — |
| | | | | like | |
| AFNX01021957 | | | 0 | DIRS- | — |
| | | | | like | |
| JNCD01001357 | | | 0 | DIRS- | — |
| | | | | like | |
| JMKM01002805 | | | 0 | DIRS- | — |
| | | | | like | |
| ABPJ01025120 | | | 0 | DIRS- | — |
| | | | | like | |
| AGTA02023338 | | | 0 | DIRS- | — |
| | | | | like | |
| HQ447060 | | | 0 | DIRS- | — |
| | | | | like | |
| GAIB01104168 | | | 0 | DIRS- | — |
| | | | | like | |
| BAHO01326816 | | | 0 | DIRS- | — |
| | | | | like | |
| AESE010643923 | | | 0 | DIRS- | — |
| | | | | like | |
| GAHO01055858 | | | 0 | DIRS- | — |
| | | | | like | |
| APWO01060904 | | | 0 | Ngaro- | — |
| | | | | like | |
| APWO01060904 | | | 0 | Ngaro- | — |
| | | | | like | |
| AHAT01041850 | | | 0 | Ngaro- | — |
| | | | | like | |
| BAAF04075296 | | | 0 | Ngaro- | — |
| | | | | like | |
| AUPQ01010767 | | | 0 | Ngaro- | — |
| | | | | like | |
| GAH001122442 | | | 0 | Ngaro- | — |
| | | | | like | |
| BAHO01173054 | | | 0 | Ngaro- | — |
| | | | | like | |
| ALBS01000010 | | | 0 | Crypton | — |
| ALBS01000010 | | | 0 | Crypton | — |
| XM_001226232 | | | 0 | Crypton | — |
| AFRE01000827 | | | 0 | Crypton | — |
| XM_002483890 | | | 0 | Crypton | — |
| XM_001239641 | | | 0 | Crypton | — |
| WP_011039584 | site-specific | MGETGRQLAVVTADADV | 371 | mrpA | 176 |
| | integrase | VKAKLVDDKTAGASVVVH | | | |
| | [Streptomyces | TDRDRHLSPETVAAIAASV | | | |
| | coelicolor] | ADSTRRAYGTDRAAFAAW | | | |
| | | CAEEDRTAVPASAETMAE | | | |
| | | WVRHLTVTPRPRTQRPAGP | | | |
| | | STIERAMSAVTTWHEEQGR | | | |
| | | PKPNMRGARAVLNAYKDR | | | |
| | | LAVEKAEAAQARQATAAL | | | |
| | | PPQIRAMLAGVDRTTLAGK | | | |
| | | RNAALVLLGFATAARVSEL | | | |
| | | VALDVDTVTEAEHGYDVT | | | |
| | | LYRKKVRKHTPNP1LYGTD | | | |
| | | PATCPVRALRAYLAALAA | | | |
| | | AGRTDGPLEVRVDRWDRL | | | |
| | | APPMTRRGRVIGDPAGRM | | | |
| | | TAEAAAEVIERLAVAAGLS | | | |
| | | GDWSGHSLRRGFATAARA | | | |
| | | AGHDPLEIARAGGWVDGS | | | |
| | | RVLARYMDDVDRVKNSPL | | | |
| | | VGIGL | | | |

### REFERENCES

¹Hacein-Bey-Abina, S., et al. (2008). “Insertional oncogenesis in 4 patients after retrovirus-mediated gene therapy of SCID-X1.” J Clin Invest 118(9): 3132-3142.

### ²McClements, M. E. and R. E. MacLaren (2017). “Adeno-associated Virus (AAV) Dual Vector Strategies for Gene Therapy Encoding Large Transgenes.” Yale J Biol Med 90(4): 611-623.

³Merrick, C. A., et al. (2016). “Rapid Optimization of Engineered Metabolic Pathways with Serine Integrase Recombinational Assembly (SIRA).” Methods Enzymol 575: 285-317.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

What is claimed is:

1. A method comprising delivering to a cell (a) a first vector comprising a first segment of a gene of interest and a first recombination site, (b) a second vector comprising a second segment of the gene of interest and a second recombination site, (c) and a cognate site-specific recombinase or a nucleic acid encoding a cognate site-specific recombinase.

2. The method of claim 1, wherein (c) is a nucleic acid encoding a cognate site-specific recombinase.

3. The method of claim 2, wherein the nucleic acid encoding a cognate site-specific recombinase is delivered on the first or second vector.

4. The method of claim 2, wherein the nucleic acid encoding a cognate site-specific recombinase is delivered on a third vector.

5. A method comprising delivering to a cell
(a) a first vector comprising a first nucleic acid comprising, optionally in a 5′ to 3′ orientation, a first promoter operably linked to a first segment of a gene of interest, a splice donor site, and a first recombination site, wherein the first nucleic acid is flanked by a first pair inverted terminal repeat sequences;
(b) a second vector comprising a second nucleic acid comprising, optionally in a 5′ to 3′ orientation, a second recombination site, a splice acceptor site, a second segment of the gene of interest, and a post-transcriptional regulator element, optionally WPRE, wherein the second nucleic acid is flanked by a second pair of inverted terminal repeat sequences; and
(c) a third vector comprising a third nucleic acid comprising a second promoter operably linked to a nucleotide sequence encoding a cognate site-specific recombinase and a post-transcriptional regulator element, optionally WPRE, wherein the third nucleic acid is flanked by a second pair of inverted terminal repeat sequences.

6. The method of any one of the preceding claims, wherein the cognate site-specific recombinase catalyzes a recombination event to join the first segment to the second segment.

7. The method of any one of the preceding claims, wherein the vector is a plasmid.

8. The method of any one of the preceding claims, wherein the vector is a viral vector.

9. The method of claim 8, wherein the viral vector is selected from the group consisting of adeno-associated viral vectors, adenoviral vectors, lentiviral vectors, and retroviral vectors

10. The method of claim 9, wherein the viral vector is an adeno-associated viral (AAV) vector, optionally an AAV2 vector.

11. The method of any one of the preceding claims, wherein the site-specific recombinase is a serine recombinase.

12. The method of claim 11, wherein the serine recombinase is selected from the group consisting of Bxb1 recombinase, TP901-1 recombinase, PhiC31 recombinase, TG1 recombinase, and PhiRv1 recombinase.

13. The method of claim 12, wherein the serine recombinase is a Bxb1 recombinase.

14. The method of any one of the preceding claims, wherein the site-specific recombinase is a tyrosine recombinase.

15. The method of claim 14, wherein the tyrosine recombinase is selected from the group consisting of Cre recombinase, Flp recombinase, XerC/D recombinase, and XerA recombinase.

16. The method of claim 15, wherein the tyrosine recombinase is Cre recombinase.

17. The method of any one of the preceding claims, wherein the first segment is a first exon of the gene of interest, and the second segment is a second exon of the gene of interest.

18. The method of any one of the preceding claims, wherein the gene of interest is a therapeutic gene of interest and/or encodes a therapeutic protein.

19. The method of any one of the preceding claims, wherein the gene of interest encodes a Cas protein, optionally a Cas9 or Cas12a protein, optionally fused to a transcriptional activator, a transcriptional repressor, or a deaminase.

20. A composition, cell, or kit comprising (a) a first vector comprising a first segment of a gene of interest and a first recombination site, (b) a second vector comprising a second segment of the gene of interest and a second recombination site, (c) and a cognate site-specific recombinase or a nucleic acid encoding a cognate site-specific recombinase.

21. A composition, cell, or kit comprising
(a) a first vector comprising a first nucleic acid comprising, optionally in a 5′ to 3′ orientation, a first promoter operably linked to a first segment of a gene of interest, a splice donor site, and a first recombination site, wherein the first nucleic acid is flanked by a first pair inverted terminal repeat sequences;
(b) a second vector comprising a second nucleic acid comprising, optionally in a 5′ to 3′ orientation, a second recombination site, a splice acceptor site, a second segment of the gene of interest, and a post-transcriptional regulator element, optionally WPRE, wherein the second nucleic acid is flanked by a second pair of inverted terminal repeat sequences; and
(c) a third vector comprising a third nucleic acid comprising a second promoter operably linked to a nucleotide sequence encoding a cognate site-specific recombinase and a post-transcriptional regulator element, optionally WPRE, wherein the third nucleic acid is flanked by a second pair of inverted terminal repeat sequences.

22. A method comprising delivering to a cell (a) a first vector comprising a first segment of a nucleic acid segment and a first recombination site, (b) a second vector comprising a second segment of the nucleic acid and a second recombination site, (c) and a cognate site-specific enzyme or a nucleic acid encoding a cognate site-specific nucleic acid-rearranging enzyme that catalyzes a recombination event to join the first segment to the second segment, thereby forming a transcription product.

23. The method of claim 22, wherein (c) comprises the nucleic acid encoding a cognate site-specific nucleic acid-rearranging enzyme that catalyzes joining of the first segment to the second segment.

24. The method of claim 22 or 23 further comprising at least one additional vector comprising at least one addition segment of the nucleic acid and at least one addition recombination site.

25. The method of any one of the preceding claims, wherein the first vector or second vector comprises the nucleic acid encoding the cognate site-specific nucleic acid-rearranging enzyme.

26. The method of any one of the preceding claims, wherein a third vector comprises nucleic acid encoding the cognate site-specific nucleic acid-rearranging enzyme.

27. The method of any one of the preceding claims, wherein the first vector comprises a promoter operably linked to the first segment of the nucleic acid.

28. The method of any one of the preceding claims, wherein the third vector comprises a promoter operably linked to the nucleic acid encoding the cognate site-specific nucleic acid-rearranging enzyme.

29. The method of any one of the preceding claims, wherein the second vector comprise a post-transcriptional regulator element (e.g., WPRE).

30. The method of any one of the preceding claims, wherein the third vector comprise a post-transcriptional regulator element (e.g., WPRE).

31. The method of any one of the preceding claims, wherein following the transcription event the transcription product comprises a scar recombination site located between the first segment and the second segment.

32. The method of any one of the preceding claims, wherein the first vector further comprises a splice donor site and the second vector comprises a branch point site and a splice acceptor site, and following a recombination event, the scar recombination site of the transcription product is flanked by (i) the splice donor site and (ii) the branch point site and the splice acceptor site.

33. The method of any one of the preceding claims, wherein the first segment, second segment, and/or at least one additional segment are exons of a gene of interest, optionally wherein the gene of interest: (a) is a therapeutic gene, optionally selected from the group consisting of any of the therapeutic genes listed in Table 1; or (b) encodes a gene-editing protein, optionally a Cas9 enzyme or a Cas9 enzyme variant (e.g., Cas9 fused to a transcriptional activator, a transcriptional repressor, or a deaminase).

34. The method of any one of the preceding claims, wherein the first vector, the second vector, and/or the at least one additional vector is a viral vector, optionally selected from the group consisting of lentiviral vectors, retroviral vectors, adenoviral vectors, and adeno-associated viral vectors.

35. The method of any one of the preceding claims, wherein the first vector, the second vector, and/or the at least one additional vector is an adeno-associated viral vector.

36. The method of any one of the preceding claims, wherein the site-specific enzyme is selected from the group consisting of site-specific recombinases, DDE transposases, DDE LTR-retrotransposases, and target-primed retrotransposases.

37. The method of any one of the preceding claims, wherein the site-specific enzyme is a site-specific recombinase (SSR) selected from the group consisting of serine recombinases, RKHRY-type recombinases, and HUH-type recombinase.

38. The method of any one of the preceding claims, wherein the SSR is a serine recombinase selected from the group consisting of small serine recombinases, large serine integrases, and IS607-like serine transposases.

39. The method of any one of the preceding claims, wherein the serine recombinase is a small serine recombinase selected from the group consisting of resolvases, invertases, and resolvase-invertases.

40. The method of any one of the preceding claims, wherein the small serine recombinase is a resolvase selected from the group consisting of Tn3 resolvase and gamma-delta resolvase.

41. The method of any one of the preceding claims, wherein the small serine recombinase is an invertase selected from the group consisting of Gin invertase and Hin invertase.

42. The method of any one of the preceding claims, wherein the small serine recombinase is a resolvase-invertase selected from the group consisting of BinT resolvase-invertase and beta resolvase-invertase.

43. The method of any one of the preceding claims, wherein the serine recombinase is a large serine recombinase selected from the group consisting of Bxb1 recombinase, TP901-1 recombinase, PhiC31 recombinase, TG1 recombinase, and PhiRv1 recombinase.

44. The method of any one of the preceding claims, wherein the SSR is Bxb1 recombinase, and the recombination sites are selected from attP and attB.

45. The method of any one of the preceding claims, wherein the SSR is a RKHRY-type recombinase selected from the group consisting of tyrosine recombinases, tyrosine integrases, tyrosine invertases, tyrosine shufflons, tyrosine transposases, topoisomerase IB, and telomere resolvases.

46. The method of any one of the preceding claims, wherein the RKHRY-type recombinase is a tyrosine recombinase selected from the group consisting of Cre recombinase, Flp recombinase, XerC/D recombinase, and XerA recombinase.

47. The method of any one of the preceding claims, wherein the RKHRY-type recombinase is a tyrosine integrase selected from the group consisting of Lambda integrase, P2 integrase, and HK022 integrase.

48. The method of any one of the preceding claims, wherein the RKHRY-type recombinase is a tyrosine invertase selected from the group consisting of FimB invertase, FimE invertase, and HbiF invertase.

49. The method of any one of the preceding claims, wherein the RKHRY-type recombinase is a tyrosine Rci shufflon.

50. The method of any one of the preceding claims, wherein the RKHRY-type recombinase is a tyrosine transposase selected from the group consisting of crypton transposases, DIR transposases, Ngaro transposases, PAT transposases, Tec transposases, Tn916 transposases, and CTnDOT transposases.

51. The method of any one of the preceding claims, wherein the SSR is a HUH-type recombinase selected from the group consisting of Y1-transposases of IS200/IS605 (e.g., IS608 TnpA and ISDra2), and ISC transposases (e.g., IscA), helitron transposases, IS91 transposases, AAV Rep78 transposases, and TrwC relaxases.

52. The method of any one of the preceding claims, wherein the site-specific enzyme is a DDE transposase selected from the group consisting of Tc1/mariner transposases, piggyBac transposases, Transib transposases, hAT transposases, Tn5 transposases, P elements, mutator transposases, and CMC transposases.

53. The method of any one of the preceding claims, wherein the site-specific enzyme is a DDE LTR-retrotransposase selected from the group consisting of Ty3/gypsy and HIV integrase.

54. The method of any one of the preceding claims, wherein the site-specific enzyme is a target-primed retrotransposase selected from the group consisting of LINE-1 and Group II introns.

55. The method of any one of the preceding claims, wherein the first vector, second vector, third vector, and/or site-specific nucleic acid-rearranging enzyme are delivered to the cell via electroporation, polymer formulation, or other transfection reagent.

56. A method comprising delivering to a cell at least two viral vectors, each comprising a payload, using a site-specific recombinase.

57. The method of claim 56, wherein the viral vectors are adeno-associated viral vectors.

58. The method of claim 56 or 57, wherein the site-specific recombinase is Bxb1 recombinase.

59. A cell comprising the first vector, the second vector, and the cognate site-specific enzyme or the nucleic acid encoding the cognate site-specific nucleic acid-rearranging enzyme of any one of the preceding claims.

60. The cell of claim 59, wherein the cell is a mammalian cell, optionally a human cell.

61. A composition comprising the first vector, the second vector, and the cognate site-specific enzyme or the nucleic acid encoding the cognate site-specific nucleic acid-rearranging enzyme of any one of the preceding claims and at least one additional reagent (e.g., cell culture media or buffer).

62. A kit comprising the first vector, the second vector, and the cognate site-specific enzyme or the nucleic acid encoding the cognate site-specific nucleic acid-rearranging enzyme of any one of the preceding claims and at least one additional reagent (e.g., cell culture media or buffer), wherein the first segment, the second segment, and/or the at least one additional segment are replaced by a multiple cloning site.

63. A vector comprising any one of the vector designs of FIG. 1.

64. A composition comprising vectors comprising the 3-vector design or the 2-vector design of FIG. 1.

65. A kit comprising vectors that comprise the 3-vector design or the 2-vector design of FIG. 1, wherein the Exon 1 and Exon 2 are each replaced by a multiple cloning site.

66. A nucleic acid vector comprising, in a 5′ to 3′ orientation, a coding region, a splice donor site, a recombination site, and optionally a 5′ LTR and a 3′ LTR.

67. The nucleic acid vector of claim 66 further comprising a promoter upstream from and operably linked to the coding region, and optionally further comprising 5′ LTR and a 3′ LTR.

68. The nucleic acid vector of claim 66 further comprising a recombination site upstream from the coding region.

69. A nucleic acid vector comprising, in a 5′ to 3′ orientation, a recombination site, a splice acceptor site, a coding region, optionally a post-transcriptional regulator element, and optionally a 5′ LTR and a 3′ LTR.

70. The nucleic acid vector of claim 69 further comprising a promoter, a recombination site, a coding region that encodes a site-specific nucleic acid-rearranging enzyme (e.g., as site-specific recombinase), and optionally a post-transcriptional regulator element, wherein the promoter is operably linked to the coding region that encodes a site-specific nucleic acid-rearranging enzyme.

71. A cell, composition, or kit comprising the nucleic acid vector of claims 68 and 70.

72. A cell, composition, or kit comprising the nucleic acid vector of claim 67 and the nucleic acid vector of claim 69.

73. The cell, composition, or kit of claim 72 further comprising a nucleic acid vector comprising, in a 5′ to 3′ orientation, a promoter operably linked to a coding region that encodes a site-specific nucleic acid-rearranging enzyme (e.g., as site-specific recombinase), optionally a post-transcriptional regulator element, optionally a 5′ LTR and a 3′ LTR, optionally a recombination site upstream from the coding region and another recombination site downstream from the coding region.
